SEQUENCE LISTING (157 sequences)

SEQ ID NO: 1 - DNA, 29 bp, synthetic construct
  source 1..29: mol_type = other DNA
tttactattaataaacctaaagggtatgt

SEQ ID NO: 2 - DNA, 24 bp, synthetic construct
  source 1..24: mol_type = other DNA
ggtatgtaggyaaggagtttcctc

SEQ ID NO: 3 - DNA, 24 bp, synthetic construct
  source 1..24: mol_type = other DNA
gatatgtcggggcggaatttccac

SEQ ID NO: 4 - DNA, 23 bp, synthetic construct
  source 1..23: mol_type = other DNA
gacaccaaagcgaaagacaacac

SEQ ID NO: 5 - DNA, 24 bp, synthetic construct
  source 1..24: mol_type = other DNA
gatacgttggacaagaattccctc

SEQ ID NO: 6 - DNA, 26 bp, synthetic construct
  source 1..26: mol_type = other DNA
gaagcctgagaaggtttctgactagg

SEQ ID NO: 7 - DNA, 24 bp, synthetic construct
  source 1..24: mol_type = other DNA
gttgccaacgccatttacgatgga

SEQ ID NO: 8 - DNA, 28 bp, synthetic construct
  source 1..28: mol_type = other DNA
gctgattacgagccgataagaactactg

SEQ ID NO: 9 - DNA, 29 bp, synthetic construct
  source 1..29: mol_type = other DNA
tttactattaataagccgaaaggatatgt

SEQ ID NO: 10 - DNA, 25 bp, synthetic construct
  source 1..25: mol_type = other DNA
taggagcttcattccaatatgctca

SEQ ID NO: 11 - DNA, 23 bp, synthetic construct
  source 1..23: mol_type = other DNA
gctttctttcgaacgagggttga

SEQ ID NO: 12 - DNA, 24 bp, synthetic construct
  source 1..24: mol_type = other DNA
gatatgtagggcaagaattccctc

SEQ ID NO: 13 - DNA, 21 bp, synthetic construct
  source 1..21: mol_type = other DNA
agactcatgaaccggagcaac

SEQ ID NO: 14 - DNA, 20 bp, synthetic construct
  source 1..20: mol_type = other DNA
ccaacgccatttacgatgga

SEQ ID NO: 15 - DNA, 24 bp, synthetic construct
  source 1..24: mol_type = other DNA
gatatgtaggyaaggagtttcctc

SEQ ID NO: 16 - DNA, 29 bp, synthetic construct
  source 1..29: mol_type = other DNA
tttactatcaataagcctaaaggatatgt

SEQ ID NO: 17 - DNA, 24 bp, synthetic construct
  source 1..24: mol_type = other DNA
cacctgacaccattccgaactcag

SEQ ID NO: 18 - DNA, 25 bp, synthetic construct
  source 1..25: mol_type = other DNA
ggaagaagacctggtcaatctatta

SEQ ID NO: 19 - DNA, 24 bp, synthetic construct
  source 1..24: mol_type = other DNA
gatatgtaggcaaggagtttcctc

SEQ ID NO: 20 - DNA, 23 bp, synthetic construct
  source 1..23: mol_type = other DNA
accaaagcgaaagacaacacttc

SEQ ID NO: 21 - DNA, 31 bp, synthetic construct
  source 1..31: mol_type = other DNA
accatttaactccaatgtarggagtgaacat

SEQ ID NO: 22 - DNA, 28 bp, synthetic construct
  source 1..28: mol_type = other DNA
gtagggagtgaacatattcagtctgtaa

SEQ ID NO: 23 - DNA, 28 bp, synthetic construct
  source 1..28: mol_type = other DNA
gctgctgcaccaccactcgtattttctg

SEQ ID NO: 24 - DNA, 26 bp, synthetic construct
  source 1..26: mol_type = other DNA
cagctccagcaatagttgggttaagc

SEQ ID NO: 25 - DNA, 24 bp, synthetic construct
  source 1..24: mol_type = other DNA
gatagctgctaatgcatggtaatg

SEQ ID NO: 26 - DNA, 21 bp, synthetic construct
  source 1..21: mol_type = other DNA
actcatgaaccggagcaacct

SEQ ID NO: 27 - DNA, 26 bp, synthetic construct
  source 1..26: mol_type = other DNA
gcttccagcctctcagccttccactc

SEQ ID NO: 28 - DNA, 28 bp, synthetic construct
  source 1..28: mol_type = other DNA
gtaaggagtgaacatatttaatctgtaa

SEQ ID NO: 29 - DNA, 23 bp, synthetic construct
  source 1..23: mol_type = other DNA
tcttctcagtctcttggataatg

SEQ ID NO: 30 - DNA, 28 bp, synthetic construct
  source 1..28: mol_type = other DNA
gtagggagtgaacatattgagtctgtaa

SEQ ID NO: 31 - DNA, 28 bp, synthetic construct
  source 1..28: mol_type = other DNA
gtctcgtcctcaccgcgctactcttaac

SEQ ID NO: 32 - DNA, 28 bp, synthetic construct
  source 1..28: mol_type = other DNA
gtaaggagtgaacatattcagtctgtaa

SEQ ID NO: 33 - DNA, 28 bp, synthetic construct
  source 1..28: mol_type = other DNA
gtagggagtgaacatattcaatctgtaa

SEQ ID NO: 34 - DNA, 24 bp, synthetic construct
  source 1..24: mol_type = other DNA
tggcgacgacctactctcccatac

SEQ ID NO: 35 - DNA, 25 bp, synthetic construct
  source 1..25: mol_type = other DNA
gtcttctcagtctcttggataatgg

SEQ ID NO: 36 - DNA, 23 bp, synthetic construct
  source 1..23: mol_type = other DNA
accacagcaaagagactcccgta

SEQ ID NO: 37 - DNA, 21 bp, synthetic construct
  source 1..21: mol_type = other DNA
ttgaytaccatgartggcaag

SEQ ID NO: 38 - DNA, 25 bp, synthetic construct
  source 1..25: mol_type = other DNA
ttaccatgartggcaagcaagttta

SEQ ID NO: 39 - DNA, 26 bp, synthetic construct
  source 1..26: mol_type = other DNA
aagcctctgatcgctgatggtactat

SEQ ID NO: 40 - DNA, 25 bp, synthetic construct
  source 1..25: mol_type = other DNA
cttgcttgccactcatggtaatcga

SEQ ID NO: 41 - DNA, 31 bp, synthetic construct
  source 1..31: mol_type = other DNA
ggcaagcgtttatatgtaagagcaagcattc

SEQ ID NO: 42 - DNA, 28 bp, synthetic construct
  source 1..28: mol_type = other DNA
tgccttaggagcccatcctgtacatgca

SEQ ID NO: 43 - DNA, 27 bp, synthetic construct
  source 1..27: mol_type = other DNA
agagtattggtggctctagaacaagtg

SEQ ID NO: 44 - DNA, 25 bp, synthetic construct
  source 1..25: mol_type = other DNA
tgccttaggagcccatcctgtacat

SEQ ID NO: 45 - DNA, 27 bp, synthetic construct
  source 1..27: mol_type = other DNA
tgggatggtagaagttataggtcttga

SEQ ID NO: 46 - DNA, 27 bp, synthetic construct
  source 1..27: mol_type = other DNA
gaaggcctcttacgtggatgagagggc

SEQ ID NO: 47 - DNA, 27 bp, synthetic construct
  source 1..27: mol_type = other DNA
ccacagcaaagagactcccgtagaccg

SEQ ID NO: 48 - DNA, 20 bp, synthetic construct
  source 1..20: mol_type = other DNA
gctacgcatacccgcgttgc

SEQ ID NO: 49 - DNA, 17 bp, synthetic construct
  source 1..17: mol_type = other DNA
gcgtgtctgaagaaggc

SEQ ID NO: 50 - DNA, 20 bp, synthetic construct
  source 1..20: mol_type = other DNA
gtcacgcatacccgcgttgc

SEQ ID NO: 51 - DNA, 20 bp, synthetic construct
  source 1..20: mol_type = other DNA
taccacccccacggcgattt

SEQ ID NO: 52 - DNA, 21 bp, synthetic construct
  source 1..21: mol_type = other DNA
tatcggaacgtaccgggtagc

SEQ ID NO: 53 - DNA, 17 bp, synthetic construct
  source 1..17: mol_type = other DNA
gttgaaacaccgcccgg

SEQ ID NO: 54 - DNA, 20 bp, synthetic construct
  source 1..20: mol_type = other DNA
actgtacgcgatgcacgagc

SEQ ID NO: 55 - DNA, 19 bp, synthetic construct
  source 1..19: mol_type = other DNA
gcgattccgcagtttacga

SEQ ID NO: 56 - DNA, 23 bp, synthetic construct
  source 1..23: mol_type = other DNA
cagcattcaatttgttccgagtc

SEQ ID NO: 57 - DNA, 19 bp, synthetic construct
  source 1..19: mol_type = other DNA
gttggatgcccgtactggg

SEQ ID NO: 58 - DNA, 20 bp, synthetic construct
  source 1..20: mol_type = other DNA
tatcggaacgtaccgggtag

SEQ ID NO: 59 - DNA, 21 bp, synthetic construct
  source 1..21: mol_type = other DNA
tatcggaacgtaccgggtagc

SEQ ID NO: 60 - DNA, 20 bp, synthetic construct
  source 1..20: mol_type = other DNA
cgaagaccttcgagcagaca

SEQ ID NO: 61 - DNA, 23 bp, synthetic construct
  source 1..23: mol_type = other DNA
gcttattcttcaggtaccgtcat

SEQ ID NO: 62 - DNA, 18 bp, synthetic construct
  source 1..18: mol_type = other DNA
gggatttcacatcctgct

SEQ ID NO: 63 - DNA, 27 bp, synthetic construct
  source 1..27: mol_type = other DNA
gaactggtttcatctgattactttcca

SEQ ID NO: 64 - DNA, 16 bp, synthetic construct
  source 1..16: mol_type = other DNA
acccacgctttcggac

SEQ ID NO: 65 - DNA, 21 bp, synthetic construct
  source 1..21: mol_type = other DNA
gtattaccgcggctgctggca

SEQ ID NO: 66 - DNA, 20 bp, synthetic construct
  source 1..20: mol_type = other DNA
accgattttgtaaggcaggg

SEQ ID NO: 67 - DNA, 20 bp, synthetic construct
  source 1..20: mol_type = other DNA
cgaagaccttcgagcagaca

SEQ ID NO: 68 - DNA, 21 bp, synthetic construct
  source 1..21: mol_type = other DNA
tcggttttgggttcgtccaaa

SEQ ID NO: 69 - DNA, 25 bp, synthetic construct
  source 1..25: mol_type = other DNA
cgaaattttgcgccatacggacgat

SEQ ID NO: 70 - DNA, 24 bp, synthetic construct
  source 1..24: mol_type = other DNA
cggtttgaccggttaaaaaaagat

SEQ ID NO: 71 - DNA, 27 bp, synthetic construct
  source 1..27: mol_type = other DNA
agccctgctatgactatcaaccctgcc

SEQ ID NO: 72 - DNA, 19 bp, synthetic construct
  source 1..19: mol_type = other DNA
ctttgaaccatcagtgaaa

SEQ ID NO: 73 - DNA, 20 bp, synthetic construct
  source 1..20: mol_type = other DNA
ggccgccgatattggcaaca

SEQ ID NO: 74 - DNA, 28 bp, synthetic construct
  source 1..28: mol_type = other DNA
cgggttgtaaaggacttttgtcagggaa

SEQ ID NO: 75 - DNA, 23 bp, synthetic construct
  source 1..23: mol_type = other DNA
aaggctgttgccaatatcggcgg

SEQ ID NO: 76 - DNA, 16 bp, synthetic construct
  source 1..16: mol_type = other DNA
gccaatatcggcggcc

SEQ ID NO: 77 - DNA, 30 bp, synthetic construct
  source 1..30: mol_type = other DNA
aatactgccttgctgatgcgtaactgccgg

SEQ ID NO: 78 - DNA, 22 bp, synthetic construct
  source 1..22: mol_type = other DNA
ttgtcagggaagaaaaggctgt

SEQ ID NO: 79 - DNA, 19 bp, synthetic construct
  source 1..19: mol_type = other DNA
cccttcaacatcagtgaaa

SEQ ID NO: 80 - DNA, 28 bp, synthetic construct
  source 1..28: mol_type = other DNA
cgttcttgacgctccatatcgctatgaa

SEQ ID NO: 81 - DNA, 18 bp, synthetic construct
  source 1..18: mol_type = other DNA
ggccgccgatattggcaa

SEQ ID NO: 82 - DNA, 21 bp, synthetic construct
  source 1..21: mol_type = other DNA
cggcaaagtggatgcaaccga

SEQ ID NO: 83 - DNA, 26 bp, synthetic construct
  source 1..26: mol_type = other DNA
cgcctatacgcctgctactttcacgc

SEQ ID NO: 84 - DNA, 26 bp, synthetic construct
  source 1..26: mol_type = other DNA
gccttttcttccctgacaaaagtcct

SEQ ID NO: 85 - DNA, 23 bp, synthetic construct
  source 1..23: mol_type = other DNA
cactcaagctcacaacaccaaca

SEQ ID NO: 86 - DNA, 25 bp, synthetic construct
  source 1..25: mol_type = other DNA
cattgataacgaagctctttacgat

SEQ ID NO: 87 - DNA, 20 bp, synthetic construct
  source 1..20: mol_type = other DNA
ccagaagtgggctacacacc

SEQ ID NO: 88 - DNA, 26 bp, synthetic construct
  source 1..26: mol_type = other DNA
cwctttacgatatytgcttccgtaca

SEQ ID NO: 89 - DNA, 17 bp, synthetic construct
  source 1..17: mol_type = other DNA
aacatccccaacatctt

SEQ ID NO: 90 - DNA, 28 bp, synthetic construct
  source 1..28: mol_type = other DNA
aaagatgggtgttttaagctagataagg

SEQ ID NO: 91 - DNA, 20 bp, synthetic construct
  source 1..20: mol_type = other DNA
gatcatgttctatcttttca

SEQ ID NO: 92 - DNA, 28 bp, synthetic construct
  source 1..28: mol_type = other DNA
aagatgggtgttttaagctagataaggt

SEQ ID NO: 93 - DNA, 22 bp, synthetic construct
  source 1..22: mol_type = other DNA
cattgaccacacggacaaaaag

SEQ ID NO: 94 - DNA, 20 bp, synthetic construct
  source 1..20: mol_type = other DNA
taatggcagaatctttggag

SEQ ID NO: 95 - DNA, 25 bp, synthetic construct
  source 1..25: mol_type = other DNA
attgtcgaacattggtcttaccctc

SEQ ID NO: 96 - DNA, 24 bp, synthetic construct
  source 1..24: mol_type = other DNA
gcatgttgtgccggacataaccat

SEQ ID NO: 97 - DNA, 19 bp, synthetic construct
  source 1..19: mol_type = other DNA
ataccaaggccggaagcac

SEQ ID NO: 98 - DNA, 18 bp, synthetic construct
  source 1..18: mol_type = other DNA
ccattcttttagaccctt

SEQ ID NO: 99 - DNA, 23 bp, synthetic construct
  source 1..23: mol_type = other DNA
cgtcttcaagtatgccccagtac

SEQ ID NO: 100 - DNA, 21 bp, synthetic construct
  source 1..21: mol_type = other DNA
gaactttaaccgaaggacttc

SEQ ID NO: 101 - DNA, 22 bp, synthetic construct
  source 1..22: mol_type = other DNA
tctgtgccgtcttcaagtatgc

SEQ ID NO: 102 - DNA, 19 bp, synthetic construct
  source 1..19: mol_type = other DNA
tgccggacataaccatgga

SEQ ID NO: 103 - DNA, 20 bp, synthetic construct
  source 1..20: mol_type = other DNA
gatcaccaccttagtttaca

SEQ ID NO: 104 - DNA, 18 bp, synthetic construct
  source 1..18: mol_type = other DNA
cgaagtgctcgaatgcga

SEQ ID NO: 105 - DNA, 21 bp, synthetic construct
  source 1..21: mol_type = other DNA
gacctctagaagaagactcag

SEQ ID NO: 106 - DNA, 25 bp, synthetic construct
  source 1..25: mol_type = other DNA
ccgaagttcatgtcctctccaagcg

SEQ ID NO: 107 - DNA, 26 bp, synthetic construct
  source 1..26: mol_type = other DNA
ccgaagttcatgtcctctccaagcgt

SEQ ID NO: 108 - DNA, 25 bp, synthetic construct
  source 1..25: mol_type = other DNA
acggcgatcttaaccaccttgtttc

SEQ ID NO: 109 - DNA, 14 bp, synthetic construct
  source 1..14: mol_type = other DNA
acggcgatcttaac

SEQ ID NO: 110 - DNA, 26 bp, synthetic construct
  source 1..26: mol_type = other DNA
agttcatgtcctctccaagcgtaagt

SEQ ID NO: 111 - DNA, 26 bp, synthetic construct
  source 1..26: mol_type = other DNA
gctaaactcgatctcggtcgagaagc

SEQ ID NO: 112 - DNA, 26 bp, synthetic construct
  source 1..26: mol_type = other DNA
aagctcacaacaccaacatacggcga

SEQ ID NO: 113 - DNA, 25 bp, synthetic construct
  source 1..25: mol_type = other DNA
tctggcaagatcaaggacatcctcc

SEQ ID NO: 114 - DNA, 25 bp, synthetic construct
  source 1..25: mol_type = other DNA
gagttagggtctaatgtttgatgtg

SEQ ID NO: 115 - DNA, 24 bp, synthetic construct
  source 1..24: mol_type = other DNA
tcatttcggatggtcaagcagcca

SEQ ID NO: 116 - DNA, 22 bp, synthetic construct
  source 1..22: mol_type = other DNA
cctccacctttcactactcatc

SEQ ID NO: 117 - DNA, 19 bp, synthetic construct
  source 1..19: mol_type = other DNA
atgctggcacacggatatt

SEQ ID NO: 118 - DNA, 24 bp, synthetic construct
  source 1..24: mol_type = other DNA
aggttcatccaccaccattctcct

SEQ ID NO: 119 - DNA, 19 bp, synthetic construct
  source 1..19: mol_type = other DNA
gcgtcgtttctccctcaaa

SEQ ID NO: 120 - DNA, 23 bp, synthetic construct
  source 1..23: mol_type = other DNA
catctccgccttataccactatc

SEQ ID NO: 121 - DNA, 25 bp, synthetic construct
  source 1..25: mol_type = other DNA
tggtgttgagcaatacgacttgggt

SEQ ID NO: 122 - DNA, 21 bp, synthetic construct
  source 1..21: mol_type = other DNA
gcggaaggatcattacagaat

SEQ ID NO: 123 - DNA, 21 bp, synthetic construct
  source 1..21: mol_type = other DNA
ggttgagtttaatctctggca

SEQ ID NO: 124 - DNA, 29 bp, synthetic construct
  source 1..29: mol_type = other DNA
actttgctttggtaggccttctatatggg

SEQ ID NO: 125 - DNA, 22 bp, synthetic construct
  source 1..22: mol_type = other DNA
ttcactgtttctgctgatcata

SEQ ID NO: 126 - DNA, 25 bp, synthetic construct
  source 1..25: mol_type = other DNA
ggctttaactaattcaataccagtc

SEQ ID NO: 127 - DNA, 24 bp, synthetic construct
  source 1..24: mol_type = other DNA
tggtggcaacacctatactggtgt

SEQ ID NO: 128 - DNA, 23 bp, synthetic construct
  source 1..23: mol_type = other DNA
ctctacgactgaaggtgctattc

SEQ ID NO: 129 - DNA, 20 bp, synthetic construct
  source 1..20: mol_type = other DNA
gcaccgaaggtcaatctcat

SEQ ID NO: 130 - DNA, 24 bp, synthetic construct
  source 1..24: mol_type = other DNA
tccactcatgcaaccatctgccaa

SEQ ID NO: 131 - DNA, 22 bp, synthetic construct
  source 1..22: mol_type = other DNA
tggatcttagcagcttgcttta

SEQ ID NO: 132 - DNA, 22 bp, synthetic construct
  source 1..22: mol_type = other DNA
gggcatgattatccctgtagtt

SEQ ID NO: 133 - DNA, 24 bp, synthetic construct
  source 1..24: mol_type = other DNA
aagaactggtgctggtggagtctt

SEQ ID NO: 134 - DNA, 23 bp, synthetic construct
  source 1..23: mol_type = other DNA
catctgcagcgtttagatctttc

SEQ ID NO: 135 - DNA, 21 bp, synthetic construct
  source 1..21: mol_type = other DNA
caagggttctggtgaacctaa

SEQ ID NO: 136 - DNA, 29 bp, synthetic construct
  source 1..29: mol_type = other DNA
tcggcaatttccttaacttggtccttagt

SEQ ID NO: 137 - DNA, 22 bp, synthetic construct
  source 1..22: mol_type = other DNA
gcagcgtttagatcttgcattt

SEQ ID NO: 138 - DNA, 23 bp, synthetic construct
  source 1..23: mol_type = other DNA
aaaggatctggcgaacctaatac

SEQ ID NO: 139 - DNA, 28 bp, synthetic construct
  source 1..28: mol_type = other DNA
tggtttcagcgatttccttaacttgagc

SEQ ID NO: 140 - DNA, 22 bp, synthetic construct
  source 1..22: mol_type = other DNA
ctggcgaacctaacactaagaa

SEQ ID NO: 141 - DNA, 19 bp, synthetic construct
  source 1..19: mol_type = other DNA
cgcattgctgcttcaacat

SEQ ID NO: 142 - DNA, 25 bp, synthetic construct
  source 1..25: mol_type = other DNA
tcgctgaaaccaagatgcaagacct

SEQ ID NO: 143 - DNA, 23 bp, synthetic construct
  source 1..23: mol_type = other DNA
gtcgtgttatgtgaatgggattg

SEQ ID NO: 144 - DNA, 19 bp, synthetic construct
  source 1..19: mol_type = other DNA
ccgcgctacttgggaataa

SEQ ID NO: 145 - DNA, 24 bp, synthetic construct
  source 1..24: mol_type = other DNA
aattcccagccgtagagggtcatg

SEQ ID NO: 146 - DNA, 22 bp, synthetic construct
  source 1..22: mol_type = other DNA
ccttaccagggcttgacattta

SEQ ID NO: 147 - DNA, 21 bp, synthetic construct
  source 1..21: mol_type = other DNA
cgggacttaacccaacatctc

SEQ ID NO: 148 - DNA, 24 bp, synthetic construct
  source 1..24: mol_type = other DNA
aaggagcctaaacaggtggtgcat

SEQ ID NO: 149 - DNA, 20 bp, synthetic construct
  source 1..20: mol_type = other DNA
aactgagacacggtccaaac

SEQ ID NO: 150 - DNA, 22 bp, synthetic construct
  source 1..22: mol_type = other DNA
ccatagagccttcatccttcac

SEQ ID NO: 151 - DNA, 24 bp, synthetic construct
  source 1..24: mol_type = other DNA
atattggtcaatgggcgctagcct

SEQ ID NO: 152 - DNA, 19 bp, synthetic construct
  source 1..19: mol_type = other DNA
gtacggtcgcaaggctaaa

SEQ ID NO: 153 - DNA, 20 bp, synthetic construct
  source 1..20: mol_type = other DNA
ggaaccatcgcagtgtatgt

SEQ ID NO: 154 - DNA, 24 bp, synthetic construct
  source 1..24: mol_type = other DNA
acgcgaagaaccttaccaaggctt

SEQ ID NO: 155 - DNA, 19 bp, synthetic construct
  source 1..19: mol_type = other DNA
agatttggacctgcgagcg

SEQ ID NO: 156 - DNA, 20 bp, synthetic construct
  source 1..20: mol_type = other DNA
gagcggctgtctccacaagt

SEQ ID NO: 157 - DNA, 23 bp, synthetic construct
  source 1..23: mol_type = other DNA
ttctgacctgaaggctctgcgcg

(19) United States
(12) Patent Application Publication
(10) Pub. No.: US 2024/0175094 A1
(43) Pub. Date: May 30, 2024

(54) COLLECTION KIT, METHOD, AND SYSTEM FOR THE DETECTION OF SEXUAL HEALTH CONDITIONS

(71) Applicant: Simple HealthKit, Inc., Fremont, CA (US)

(72) Inventors: Sheena Ruby Menezes, Los Gatos, CA (US); Jun Ni, San Jose, CA (US); Jurek Majka, San Jose, CA (US); Linus Aranha, San Jose, CA (US)

(21) Appl. No.: 18/490,636

(22) Filed: Oct. 19, 2023

Related U.S. Application Data
(60) Provisional application No. 63/417,976, filed on Oct. 20, 2022.
(60) Provisional application No. 63/506,156, filed on Jun. 5, 2023.

(51) Int. Cl. C12Q 1/689; A61B 10/02; C12Q 1/6806; C12Q 1/6851; C12Q 1/6893

(52) U.S. Cl. CPC C12Q 1/689; C12Q 1/6851; C12Q 1/6893; A61B 10/02; C12Q 1/6806; C12Q 2600/16

(57) ABSTRACT

The present disclosure provides multiplexed system with methods using a multiplex real-time polymerase chain reaction (qPCR), at-home collection and laboratory testing kits for detecting whether at least one or more sexual health conditions is present in a sample. For example, the multiplex real-time qPCR method can be used for simultaneous detection of target nucleic acids (e.g., DNA) from, for example, Chlamydia trachomatis (CT), Neisseria gonorrhoeae (NG), and Trichomonas vaginalis (TV) from clinician-collected or at-home self-collected specimens. The present disclosure also computer implemented methods and non-transitory computer-readable storage medium storing executable computer program constructions comprising code for carrying out steps used in a telehealth platform for communication to and from a client device, provider device, and a pharmacy device.

### CROSS-REFERENCE TO RELATED APPLICATIONS

The present application claims the benefit of and priority to U.S. Provisional Application No. 63/417,976 filed on Oct. 20, 2022, and U.S. Provisional Application No. 63/506,156 filed on Jun. 5, 2023, both of which are incorporated by reference in their entirety.

### INCORPORATION BY REFERENCE OF SEQUENCE LISTING FILE

The present application incorporates by reference the material in the XML Sequence Listing file entitled “57600US_CRF_sequencelisting.xml” created on Jan. 24, 2024, and sized 139,704 bytes.

### BACKGROUND

The invention(s) described relate generally to multiplexed, real time systems, methods, and kits for reliably and efficiently assessing sexual health of a subject. In particular, the systems, methods and kits described herein are for detection of sexually transmitted disease (STD) or sexually transmitted infection (STI) from clinician-collected or at-home self-collected specimens suspected to contain bacteria or parasites leading to an STI or STD.

The Centers for Disease Control and Prevention (CDC) estimates that one in five people in the United States had a sexually transmitted infection (STI) in 2018 [CDC: Sexually Transmitted Infections Prevalence, Incidence, and Cost Estimates in the United States). Most recent CDC annual report “Sexually Transmitted Disease Surveillance, 2020” revealed that the cases of sexually transmitted disease (STD) continued to surge in the face of a pandemic, with 2.4 million cases of STDs reported in the U.S. in 2020 (CDC: Sexually Transmitted Disease Surveillance, 2020).

STDs are treatable. Some of them are curable, including gonorrhoea, chlamydia and trichomoniasis, but if left undiagnosed or untreated, STDs can cause significant negative long-term health impact including pelvic inflammatory disease (PID) and infertility in women, miscarriage or newborn death, and increased risk of certain cancers and HIV infection (WHO: Sexually trarsmitted infections (STIs) (www.who.int/news-room/fact-sheets/detail/sexually-transmitted-infections-(stis)), National Academies of Sciences, Engineering, and Medicine. 2021. Sexually Transmitted Infections: Adopting a Sexual Health Paradigm. Washington, DC: The National Academies Press. https://doi.org/10.17226/25955). STDs can affect anyone who is sexually active, but due to the underneath inequality of the U.S. society, people of color and marginalized LGBTQ groups—lesbian, gay, bisexual, transgender, and queer—are hit the hardest (National Academies of Sciences, Engineering, and Medicine. 2021. Sexually Transmitted Infections: Adopting a Sexual Health Paradigm. Washington, DC: The National Academies Press. https://doi.org/10.17226/25955). Each year, STDs cost billions of dollars in direct medical costs (CDC: Sexually Transmitted Infections Prevalence, Incidence, and Cost Estimates in the United States). STDs may be asymptomatic or may have symptoms that can have other causes. Developing advanced accurate and sensitive molecular diagnostic tools is critical for STD management and surveillance in the fighting to reduce STD prevalence and morbidity.

Chlamydia trachomatis is an obligate intracellular gram-negative bacterium behind chlamydia infection, the most common notifiable sexually transmitted infection in the United States (CDC: Sexually Transmitted Disease Surveillance, 2020). Early-stage Chlamydia infections are usually asymptomatic, even later-stage infections may have very mild symptoms easy to overlook. Screening coverage is important to Chlamydia trachomatis discovery. CDC recommends chlamydia screening for sexually active women aged 25 or younger, pregnant women, and women and men at high risk. Cell culture was a long-time reference diagnosis gold standard for Chlamydia trachomatis until improvements in nucleic acid amplification tests (NAATs) started to provide performance more sensitive, more specific and with ease of specimen transport. Since 2014, CDC has been recommending laboratories use NAATs to detect Chlamydia except in certain cases (CDC: Recommendations for the Laboratory-Based Detection of Chlamydia trachomatis and Neisseria gonorrhoeae—2014).

In 2020, gonorrhea was the second most notifiable sexually transmitted infection in the United States, with a total case number of 677,769 reported to CDC, a 111% increase at reporting rates since 2009 (CDC: Sexually Transmitted Disease Surveillance, 2020). Gonococcal infections commonly appear in the genital tract but can affect many sites of the body including rectum, eyes, throat and joints. However, it typically causes no symptoms in women until complications such as PID develop. Annual screening is recommended for sexually active women younger than 25 and for older women at increased risk of infection, and men who have sex with men, as well as their partners (CDC: Recommendations for the Laboratory-Based Detection of Chlamydia trachomatis and Neisseria gonorrhoeae—2014). Gonorrhea infection is caused by sexually transmitted bacterium Neisseria gonorrhoeae. Isolation and identification of N. gonorrhoeae by culture requires stringent collection and transportation conditions and the turn-over time is usually slow, making it less idea for routine surveillance of the population.

Trichomoniasis is the most prevalent non-viral STD caused by infection of a protozoan parasite Trichomonas vaginalis. It is a common curable STD however only 30% of the infections develop any symptoms. Without treatment, trichomoniasis can last for months to years. People with trichomoniasis are at a higher risk of getting or spreading other STDs; trichomoniasis in pregnant women is linked to preterm delivery (Trichomoniasis—CDC Fact Sheet). Traditionally, trichomoniasis can be diagnosed by wet mount or culture. Wet mount of motile TV on slides and visualized by microscope is quick and inexpensive, however, sensitivity ranges from 36-70% at best, depending on how skillful the evaluator is and how promptly the slide is prepared and observed (ASSOCIATION OF PUBLIC HEALTH LABORATORIES: Advances in Laboratory Detection of Trichomonas Vaginalis (Updated)). Culture diagnosis of trichomoniasis is specific but not widely available and the sensitivity is not very high (75-85%) (ASSOCIATION OF PUBLIC HEALTH LABORATORIES: Advances in Laboratory Detection of Trichomonas Vaginalis (Updated)).

The present invention addresses the needs for routine screening and detection of CT, NG and TV with an easy-to-use at home-kit and NAAT assay that can apply to both clinician-collected and at-home self-collected samples. A digital health platform enables end-to-end service for users from sample collection to obtaining medical solutions and provides sexual health condition monitoring for people in need.

For high-risk group, e.g. asymptomatic men who have sex with men, infections could be missed if diagnosis is based on a single sampling site (ref: Marcus, Julia L. MPH*; Bernstein, Kyle T. PhD, ScM*†; Kohn, Robert P. MPH*; Liska, Sally DrPH*; Philip, Susan S. MD, MPH*. Infections Missed by Urethral-Only Screening for Chlamydia or Gonorrhea Detection Among Men Who Have Sex With Men. Sexually Transmitted Diseases: October 2011—Volume 38—Issue 10—p 922-924). The system described here is versatile in performing screening with the ability to multiplex samples from different matrix, which could greatly reduce the cost of screening, increase the chance of detecting infections, especially extragenital infections, and decrease the burden of sexually transmitted disease.

### SUMMARY

This disclosure features a multiplex real-time polymerase chain reaction (qPCR) method and a system and kits for detecting the status of one or more sexual health conditions. In particular, the multiplex real-time qPCR method can be used for simultaneous detection of nucleic acid (e.g., DNA) from Chlamydia trachomatis (CT), Neisseria gonorrhoeae (NG), and Trichomonas vaginalis (TV) from clinician-collected (e.g. at a clinic or doctor's office) or at-home self-collected specimens. This disclosure also features multiplexed real-time qPCR methods used for detection of a nucleic acid (e.g., DNA) associated with one of Chlamydia trachomatis (CT), Neisseria gonorrhoeae (NG), and Trichomonas vaginalis (TV). These methods enable rapid and reliable assessment of one or more sexual health conditions by providing users who need to test for multiple health conditions an all-in-one test to easily and accessibly test for multiple sexual health conditions. The method(s) described herein include system components, assay materials, and additional elements for enabling rapid and reliable characterizations of two or more sexual health conditions of a subject in a multiplexed manner with reduced error and contamination.

Provided herein are systems, methods and kits that can efficiently test for whether at least one sexual health condition is present in a user, and providing the user with a status of whether the user has a sexual health condition via telemedicine. The systems, methods, and kits provided herein can detect whether the user has infections associated with the following agents in multiplexed formats: Chlamydia trachomatis, Neisseria gonorrhoeae, and Trichomonas vaginalis., Treponema pallidum, Gardnerella vaginitis, Candida Albicans, Mycoplasma genitalium, human immunodeficiency virus, human papillomavirus infection, Hepatitis B, and herpes simplex virus. The systems, methods, and kits provided herein can detect whether the user has infections associated with the following agents in multiplexed formats: Candida albicans, Candida dupliniensis, Candida parapsilosis, Candida tropicana, Candida Africana, Lactobacillus crispatus, Lactobacillus graseii, Lactobacillus inner, Lactobacillus jenseni, Gardnerella vaginalis, Apodium vaginae, Bactoroides fragilis, and Mobiliscus curtiis.

Provided herein are methods and kits for detecting two or more target nucleic acid sequences each associated with a sexual health condition, where the method achieves a sensitivity, accuracy, and limit of detection for determining the presence of each target that are comparable to or outperform similar methods. For example, some embodiments of detecting two or more target nucleic acid sequences enable limit of detection for determining the presence of each target is comparable to, or even lower than, a monoplex assay designed to detect the presence of a single target nucleic acid associated with a sexual health condition.

The present inventors developed a highly sensitive and specific multiplex qPCR developed for detecting Chlamydia trachomatis, Neisseria gonorrhoeae, Trichomonas vaginalis, Candida albicans, Candida dupliniensis, Candida parapsilosis, Candida tropicana, Candida Africana, Lactobacillus crispatus, Lactobacillus graseii, Lactobacillus inner, Lactobacillus jenseni, Gardnerella vaginalis, Apodium vaginae, Bactoroides fragilis, and Mobiliscus curtiis.

The methods described herein for detecting two or more target nucleic acid sequences each associated with a sexual health condition, e.g., a multiplexed assay, was found to be highly specific (NPA>99% for each pathogen) and maintained high sensitivity (PPA>95% for each pathogen) towards detecting the specific sexual health condition, such as, Chlamydia trachomatis, Neisseria gonorrhoeae, and Trichomonas vaginalis.

Provided herein is a system that provides an easy-to-use at-home kit for collecting samples from a user at-home or at a clinic, and optionally, as part of the at-home kit, or separately as a laboratory kit, additional components, that when used together, allows for qualitative analysis of the sample for detecting the presence or absence of at least one sexual health condition. The system can also include a computer readable medium (e.g., computer-readable medium storing code) for that when executed by a processor, cause the processor to receive and display results of analysis to the user via an online portal. The online portal provides access to the user and a healthcare provider for communicating results and provide an option follow up telehealth care, prescription and medication for patient care.

Clause 1. A multiplexing system for detecting whether at least one sexual health condition is present in a user, the system comprising: (a) a collection kit comprising: (i) a sample collection device for receiving one or more samples collected by the user at-home or in a clinic; (ii) a sample transport container designed for receiving the one or more samples collected at home or in a clinic; and (iii) a labeled shipping container for shipping the one or more samples to a laboratory for analysis; (b) a laboratory analysis kit comprising: (i) an amplification reaction mixture; and (ii) primers selected from: a first pair of primers that bind specifically to a target nucleic acid sequence from Chlamydia trachomatis where present in the sample, a second pair of primers that bind specifically to a target nucleic acid sequence from Neisseria gonorrhoeae, where present in the sample, a third pair of primers that bind specifically to a target nucleic acid sequence from Trichomonas vaginalis where present in the sample, or a combination thereof; (c) analyzing the sample using the laboratory analysis kit to detect whether the sexual health condition is present in the user; and (d) a computer-readable medium storing code that, when executed by a processor, causes the processor to perform the following steps: receiving and displaying results of the analysis with the laboratory analysis kit to the user, the results providing information about whether or not the user has the sexual health condition.

Clause 2. The multiplexing system of clause 1, wherein laboratory analysis kit further comprises an extraction and/or storage buffer.

Clause 3. The multiplexing system of any of clauses 1-2, wherein the analysis comprises means for nucleic acid purification and amplification.

Clause 4. The multiplexing system of any of clauses 1-3, wherein, if at least one sexual health condition is detected in the user, the processor is configured to further perform the following steps: provide telemedicine access to the user with a healthcare provider, wherein telemedicine access allows for communication with the healthcare provider for running tests, displaying test results, providing telehealth visits, prescribing and facilitating delivery of a medication.

Clause 5. The multiplexing system of any of clauses 1-4, wherein the sexual health condition is an infection associated with Chlamydia trachomatis.

Clause 6. The multiplexing system of clause 5, wherein the Chlamydia trachomatis infections are associated with Chlamydia trachomatis strains selected from Serovar D, Serovar E, Serovar F, Serovar G, Serovar H, Serovar I, Serovar J, Serovar K and LGV strains.

Clause 7. The multiplexing system of any of clauses 1-6, wherein the sexual health condition is an infection associated with Neisseria gonorrhoeae.

Clause 8. The multiplexing system of clause 7, wherein the Neisseria gonorrhoeae infections are associated with Neisseria gonorrhoeae strains selected from AR-0166, AR-0168, AR-0205, AR-0206, AR-0207, AR-0208, AR-0209, AR-0210, AR-0211, AR-0212, AR-0213, AR-0214, CDC Ng-98, and CDC Ng-116.

Clause 9. The multiplexing system of any of clauses 1-8, wherein the sexual health condition is an infection associated with Trichomonas vaginalis.

Clause 10. The multiplexing system of clause 9, wherein the Trichomonas vaginalis infections are associated with Trichomonas vaginalis strains selected from 165307-1, JH 32A #2, 11769, NYH 286, RU 357, MT87, HsD:NIH, TVC, and NYH 286.

Clause 11. The multiplexing system of clause 2, wherein the extraction and/or storage buffer comprises phosphate buffered saline, Tris-buffered saline, HEPES buffered saline, a reducing agent, sodium hydroxide, Triton X-100, octyl glucoside, PreservCyt® solution, CytoLyt® solution, or a methanol-based reagent.

Clause 12. The multiplexing system of any of clauses 1-11, wherein analysis is performed as a single reaction or in a single reaction container comprising the amplification reaction mixture, the first pair of primers, the second pair of primers, and the plurality of target nucleic acid sequences from the one or more samples.

Clause 13. The multiplexing system of clause 12, wherein, of the first pair of primers and the second pair of primers, each includes a probe, wherein the probe binds specifically to the respective target nucleic acid sequence.

Clause 14. The multiplexing system of clause 13, wherein: a first forward primer of the first pair, a second forward primer of the second pair, or each of the first and the second forward primers comprises a concentration ranging from 0-05-1 uM; or a first reverse primer of the first pair, a second reverse primer of the second pair, or each of the first and the second reverse primers comprises a concentration ranging from 0-05-1 uM.

Clause 15. The multiplexing system of clause 6, wherein the first pair of primers comprise a forward primer having a nucleotide sequence selected from SEQ ID NO: 1-19, a reverse primer having a nucleotide sequence selected from SEQ ID NO: 20-35, and a probe having a nucleotide sequence selected from SEQ ID NO: 36-47.

Clause 16. The multiplexing system of clause 8, wherein the second pair of primers comprise a forward primer having a nucleotide sequence selected from SEQ iD NOs: 48-59, a reverse primer having a nucleotide sequence selected from SEQ ID NO: 60-70, and a probe having a nucleotide sequence selected from SEQ iD NOs: 71-84.

Clause 17. The multiplexing system of clause 10, wherein the third pair of primers comprise a forward primer having a nucleotide sequence selected from SEQ ID NO: 85-95, a reverse primer having a nucleotide sequence selected from SEQ ID NO: 96-104, and a probe having a nucleotide sequence selected from SEQ ID NO: 105-115.

Clause 18. The multiplexing system of any of clauses 1-17, wherein the collection kit further comprises a sample collection tool selected from a group consisting of: a collapsible cup, a tube, a pipette, a swab, a dropper, and a combination thereof.

Clause 19. The multiplexing system of any of clauses 1-18, wherein the one or more samples are selected from a group consisting of: a urine sample, a rectal sample, a vaginal sample, a penile sample, an oral sample, and a blood sample.

Clause 20. The multiplexing system of clause 19, wherein the system comprises a combination of samples, wherein each sample of the combination of samples is collected from the user using a separate sample collection device.

Clause 21. A method for multiplex detection of whether at least one sexual health condition is present in a subject, the method comprising: (a) receiving one or more samples acquired directly by a user or indirectly by a clinician; (b) amplifying a target nucleic acid sequence of a plurality of target nucleic acid sequences from the sample by PCR amplification using primers and an amplification reaction mixture, wherein the primers are selected from: (i) a first pair of primers that bind specifically to a target nucleic acid sequence from Chlamydia trachomatis, (ii) a second pair of primers that bind specifically to a target nucleic acid sequence from Neisseria gonorrhoeae, (iii) a third pair of primers that bind specifically to a target nucleic acid sequence from Trichomonas vaginalis, or (iv) a combination thereof (c) detecting the presence or absence of a PCR amplification product from the PCR amplification, thereby determining status of the sexual health condition; (d) displaying test results to the subjec€and (e) communicating the test results to a healthcare provider.

Clause 22. The method of clause 21, wherein the method comprises, before step (b), extracting or purifying a plurality of target nucleic acids from the sample.

Clause 23. The method of any of clauses 21-22, wherein: extracting the plurality of target nucleic acids from the sample comprises extracting the plurality of target nucleic acids from the sample using a buffer, or purifying the plurality of target nucleic acids from the sample comprises using spin column-based nucleic acid purification.

Clause 24. The method of clause 23, wherein the buffer comprises phosphate buffered saline, Tris-buffered saline, HEPES buffered saline, a reducing agent, sodium hydroxide, Triton X-100, octyl glucoside, PreservCyt® solution, CytoLyt® solution, or a methanol-based reagent.

Clause 25. The method of any of clauses 21-24, wherein the amplifying step occurs in a single reaction well or as a single reaction comprising the amplification reaction mixture, the first pair of primers, the second pair of primers, the third pair of primers, and the plurality of target nucleic acid sequences from the sample.

Clause 26. The method of any of clauses 21-25, wherein the first pair of primers, the second pair of primers, and the third pair of primers, each include a probe, wherein the probe binds specifically to the respective target nucleic acid sequence.

Clause 27. The method of any of clauses 21-26, wherein: a first forward primer of the first pair, a second forward primer of the second pair, a third forward primer of the third pair, or each of the first, the second, and the third forward primers comprises a concentration ranging from 0.05-1 uM; and wherein a first reverse primer of the first pair, a second reverse primer of the second pair, a third reverse primer of the third pair, or each of the first, the second, and the third reverse primers comprises a concentration ranging from 0.05-1 uM; or the first pair of primers, the second pair of primers, the third pair of primers, the probes, or a combination thereof include detectably labeled primers or probes, wherein the probes bind specifically to the respective target nucleic acid sequences.

Clause 28. The method of any of clauses 21-27, wherein the first pair of primers comprise a forward primer having a nucleotide sequence selected from SEQ ID NO: 1-19, a reverse primer having a nucleotide sequence selected from SEQ ID NO: 20-35, and a probe having a nucleotide sequence selected from SEQ ID NO: 36-47.

Clause 29. The method of any of clauses 21-28, wherein the second pair of primers comprise a forward primer having a nucleotide sequence selected from SEQ ID NOs: 48-59, a reverse primer having a nucleotide sequence selected from SEQ ID NO: 60-70, and a probe having a nucleotide sequence selected from SEQ ID NOs: 71-84.

Clause 30. The method of any of clauses 21-29, wherein the third pair of primers comprise a forward primer having a nucleotide sequence selected from SEQ ID NO: 85-95, a reverse primer having a nucleotide sequence selected from SEQ ID NO: 96-104, and a probe having a nucleotide sequence selected from SEQ ID NO: 105-115.

Clause 31. The method of any of clauses 21-30, wherein the sample comprises one or more samples selected from: a urine sample, a vaginal sample, a rectal sample, a penile sample, and an oral sample.

Clause 32. The method of any of clauses 21-31, wherein the method does not comprise extracting the target nucleic acid sequence from the sample.

Clause 33. A non-transitory computer-readable storage medium storing executable computer program instructions, the computer program instructions comprising code for performing steps of: (a) receiving, from a client device, registration information about a user and information about a kit registered to the user for detecting status of one or more sexual health conditions; (b) providing, to the client device, status information indicating that the user has tested positive for one or more sexual health conditions; (c) responsive to the status information indicating that the user has tested positive, providing, to the client device, a questionnaire and a plurality of options selected from: a telehealth consult with a clinician; an order prescription request; or a combination thereof; (d) receiving, from the client device, input related to the selection options; and (e) providing, to the client device, a treatment care option based on input received related to the selected option.

Clause 34. The computer-readable storage medium of clause 33, wherein if the order prescription request is received from the client device, providing, to a client device, approval or denial of the order prescription request.

Clause 35. The computer-readable storage medium of clause 34, wherein if the order prescription request is approved: providing, to the client device, a prescription order of a medication and instructions for use of the medication to the user, prescription order delivery options.

Clause 36. The computer-readable storage medium of any one of clauses 34-35, wherein if the order prescription request is approved, receiving, from a pharmacy device, confirmation of the prescription details.

Clause 37. The computer-readable storage medium of any one of clauses 34-36, wherein if the order prescription request is approved, receiving, from the client device, a payment request.

Clause 38. The computer-readable storage medium of any one of clauses 33-37, before step (b), the steps further comprising providing, to the client device, one or more status updates on a laboratory test result for one or more sexual health conditions.

Clause 39. The computer-readable storage medium of any one of clauses 33-38, wherein the clinician is selected from: a nurse practitioner, a physician's assistant, and a physician.

Clause 40. The computer-readable storage medium of any one of clauses 33-39, the steps further comprising, after providing, to the client device, the questionnaire, receiving, from the client device, a response to the questionnaire.

Clause 41. The computer-readable storage medium of clause 40, wherein the response to the questionnaire comprises eligibility information about the user.

Clause 42. The computer-readable storage medium of clause 41, wherein eligibility information comprises treatment preferences and/or symptom information from the user.

Clause 43. The computer-readable storage medium of any one of clauses 33-42, wherein after receiving, from the client device, the order prescription request: providing, to the client device, status updates regarding the approval or denial of the order prescription request.

Clause 44. The computer-readable storage medium of clause 41, wherein eligibility information about the user comprises medical history about the user.

Clause 45. The computer-readable storage medium of clause 41, wherein eligibility information about the user comprises instructions to the user to provide the user's date of birth and address.

Clause 46. The computer-readable storage medium of clause 41, wherein eligibility information about the user comprises information about one or more of the following: whether or not the user is pregnant; whether or not the user is breastfeeding; whether or not the user is allergic to one or more medications; whether or not the user is taking antibiotics; whether or not the user is taking birth control; and a list of current medications.

Clause 47. The computer-readable storage medium of clause 46, wherein if the user is pregnant, during the telehealth consult with the clinician, the clinician will provide alternative medication options to the user or route the user to a primary care physician.

Clause 48. The computer-readable storage medium of any one of clauses 46-47, wherein if the user is breastfeeding, during a telehealth consult with the clinician, the clinician will provide alternative medication options to the user or route the user to a physician.

Clause 49. The computer-readable storage medium of any one of clauses 46-48, wherein if the user has one or more allergies to one or more medications, during the telehealth consult with the clinician, the clinician will provide alternative medication options to the user or route the user to a physician.

Clause 50. The computer-readable storage medium of any one of clauses 46-49, wherein if the user is taking birth control, during the telehealth consult with the clinician, the clinician will provide alternative medication options to the user or route the user to a primary care physician.

Clause 51. The computer-readable storage medium of any one of clauses 46-50, wherein if the user is taking one or more medications, during the telehealth consult with the clinician, the clinician will provide alternative medication options to the user or route the user to a primary care physician.

Clause 52. The computer-readable storage medium of clause 36, wherein the prescription details comprises shipping information of the prescription to the user's location.

Clause 53. The computer-readable storage medium of clause 36, wherein the prescription details comprise pickup information of the prescription from a local pharmacy.

Clause 54. The computer-readable storage medium of any one of clauses 41-53, wherein eligibility information comprises identification information about the user.

Clause 55. The computer-readable storage medium of any one of clauses 33-54, wherein the registration information about the user comprises providing a registration process to the client device, the registration process comprising a registration questionnaire to the user to answer screening questions and indicate information about the user.

Clause 56. The computer-readable storage medium of clause 55, wherein the registration questionnaire about the user comprises instructions to the user to provide user payment information.

Clause 57. The computer-readable storage medium of any one of clauses 55-56, wherein the registration questionnaire about the user comprises instructions to the user to provide user healthcare insurance information.

Clause 58. The computer-readable storage medium of any one of clauses 55-57, wherein the registration questionnaire about the user comprises instructions to the user to provide a sample collection time.

Clause 59. The computer-readable storage medium of any one of clauses 55-58, wherein the registration questionnaire about the user comprises instructions to the user to provide the sample ID, test name, sample collection date, and sample collection time.

Clause 60. The computer-readable storage medium of clause 44, wherein history about the user comprises instructions to the user to provide whether or not the user has tested positive for one or more sexual health conditions.

Clause 61. The computer-readable storage medium of clause 44, wherein history about the user comprises instructions to the user to provide information about the user's sexual history.

Clause 62. The computer-readable storage medium of any one of clauses 33-55, wherein registration questionnaire about the user further comprises providing the information about the user to the healthcare provider device, wherein the healthcare provider device is used by a healthcare provider.

Clause 63. The computer-readable storage medium of clause 62, wherein the healthcare provider is a physician.

Clause 64. The computer-readable storage medium of any one of clauses 33-63, wherein the code is executed by one or more processors to cause the one or more processors to perform the steps of the code.

Clause 65. The computer-readable storage medium of any one of clauses 33-64, the steps further comprising, before step (b), receiving, PCR results related to the sample ID.

Clause 66. The computer-readable storage medium of clause 65, the steps further comprising: analyzing the PCR results to determine the status information indicating that the user has tested positive for one or more sexual health conditions.

Clause 67. The computer-readable storage medium of clause 66, wherein analyzing the PCR results comprises: determining, for each amplification curve associated with a target nucleic acid sequence related to one sexual health condition, whether the amplification curve indicates a minimum threshold concentration of the target nucleic acid sequence.

Clause 68. The computer-readable storage medium of clause 67, wherein the sexual health conditions include Chlamydia trachomatis, Neisseria gonorrhoeae, Trichomonas vaginalis, or some combination thereof.

Clause 69. A computer implemented method for providing telehealth medicine and status of one or more sexual health conditions to a user, the method comprising: (a) receiving, from a client device, registration information about a user and information about a kit registered to the user for detecting status of one or more sexual health conditions; (b) providing, to the client device, status information indicating that the user has tested positive for one or more sexual health conditions; (c) responsive to the status information indicating that the user has tested positive, providing, to the client device, a questionnaire and a plurality of options selected from: a telehealth consult with a clinician; an order prescription request; or a combination thereof; (d) receiving, from the client device, input related to the selection options; and providing, to the client device, a treatment care option based on input received related to the selected option.

Clause 70. A system comprising: a computer processor; and a non-transitory computer-readable storage medium storing instructions that, when executed by the computer processor, cause the computer processor to perform the computer-implemented method of clause 69.

Clause 71. A system for detecting whether at least one sexual health condition is present in a user, the system comprising: (a) a collection kit comprising: (i) a sample collection device for receiving one or more samples collected by the user at-home or in a clinic, (ii) a sample transport container designed for receiving the one or more samples collected at home or in a clinic, and (iii) a labeled shipping container for shipping the one or more samples to a laboratory for analysis, and (b) a laboratory analysis kit comprising: (i) a buffer for extraction and/or storage of nucleic acid molecules in the sample collection device, (ii) an amplification reaction mixture, and (iii) primers selected from: a first pair of primers that bind specifically to a target nucleic acid sequence from Chlamydia trachomatis where present in the sample, a second pair of primers that bind specifically to a target nucleic acid sequence from Neisseria gonorrhoeae, where present in the sample, a third pair of primers that bind specifically to a target nucleic acid sequence from Trichomonas vaginalis where present in the sample, or a combination thereof.

Clause 72. The system of clause 71, wherein the system further comprises a thermocycler for nucleic acid purification and amplification.

Clause 73. The system of clause 72, wherein the thermocycler is configured to perform amplification as a single reaction or in a single reaction container comprising the amplification reaction mixture, the primers, and the plurality of target nucleic acid sequences from the one or more samples.

Clause 74. The system of any of clauses 71-73, wherein the system further comprises a computing device configured to provide telemedicine access to the user with a healthcare provider, wherein telemedicine access allows for communication with the healthcare provider for running tests, displaying test results, providing telehealth visits, prescribing and facilitating delivery of a medication.

Clause 75. The system of any of clauses 71-74, wherein the sexual health condition is an infection associated with Chlamydia trachomatis.

Clause 76. The system of clause 75, wherein the Chlamydia trachomatis infections are associated with Chlamydia trachomatis strains selected from Serovar D, Serovar E, Serovar F, Serovar G, Serovar H, Serovar I, Serovar J, Serovar K and LGV strains.

Clause 77. The system of any of clauses 71-76, wherein the sexual health condition is an infection associated with Neisseria gonorrhoeae.

Clause 78. The system of clause 77, wherein the Neisseria gonorrhoeae infections are associated with Neisseria gonorrhoeae strains selected from AR-0166, AR-0168, AR-0205, AR-0206, AR-0207, AR-0208, AR-0209, AR-0210, AR-0211, AR-0212, AR-0213, AR-0214, CDC Ng-98, and CDC Ng-116.

Clause 79. The system of any of clauses 71-78, wherein the sexual health condition is an infection associated with Trichomonas vaginalis.

Clause 80. The system of clause 79, wherein the Trichomonas vaginalis infections are associated with Trichomonas vaginalis strains selected from 165307-1, JH 32A #2, 11769, NYH 286, RU 357, MT87, HsD:NIH, TVC, and NYH 286.

Clause 81. The system of any of clauses 71-80, wherein the buffer comprises phosphate buffered saline, Tris-buffered saline, HEPES buffered saline, a reducing agent, sodium hydroxide, Triton X-100, octyl glucoside, PreservCyt® solution, CytoLyt® solution, a methanol-based reagent, or some combination thereof.

Clause 82. The system of any of clauses 71-81, wherein each pair of primers includes a probe that binds specifically to the respective target nucleic acid sequence.

Clause 83. The system of any of clauses 71-82, wherein: a forward primer of each pair of primers comprises a concentration ranging from 0.05-1 uM; or a reverse primer of each pair of primers comprises a concentration ranging from 0.05-1 uM.

Clause 84. The system of any of clauses 71-83, wherein the first pair of primers comprise a forward primer having a nucleotide sequence selected from SEQ ID NO: 1-19, a reverse primer having a nucleotide sequence selected from SEQ ID NO: 20-35, and a probe having a nucleotide sequence selected from SEQ ID NO: 36-47.

Clause 85. The system of any of clauses 71-84, wherein the second pair of primers comprise a forward primer having a nucleotide sequence selected from SEQ ID NOs: 48-59, a reverse primer having a nucleotide sequence selected from SEQ ID NO: 60-70, and a probe having a nucleotide sequence selected from SEQ ID NOs: 71-84.

Clause 86. The system of any of clauses 71-85, wherein the third pair of primers comprise a forward primer having a nucleotide sequence selected from SEQ ID NO: 85-95, a reverse primer having a nucleotide sequence selected from SEQ ID NO: 96-104, and a probe having a nucleotide sequence selected from SEQ ID NO: 105-115.

Clause 87. The system of any of clauses 71-86, wherein the collection kit further comprises a sample collection tool selected from a group consisting of: a collapsible cup, a tube, a pipette, a swab, a dropper, and a combination thereof.

Clause 88. The system of any of clauses 71-87, wherein the one or more samples are selected from a group consisting of: a urine sample, a rectal sample, a vaginal sample, a penile sample, an oral sample, and a blood sample.

### BRIEF DESCRIPTION OF THE DRAWINGS

FIGS. 1A-1B illustrates a system for collecting a sample directly from a subject to be used in assessing health of the subject, according to one embodiment.

FIG. 2 illustrates an example workflow of a sexual health detection system of one embodiment of the present disclosure.

FIG. 3 illustrates an example of a telehealth portal of one embodiment of the sexual health detection kit and system of the present disclosure. The user application interface includes the interface as seen by the client on a client device.

FIG. 4 provides a system environment of a platform system for providing telehealth medicine for the user according to one embodiment.

FIG. 5 shows a block diagram of the system architecture of a platform system of FIG. 1, according to one embodiment.

The figures depict various embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION

This disclosure features systems, kits and methods for detecting status of at least one sexual health condition or for detecting whether at least one sexual health condition is present in a user. In some embodiments, the systems, methods and kits can be configured to indicate statuses of different sexual health conditions in a monoplexed and/or a multiplexed manner. For example, for each of a set of sexual health conditions (e.g., STD), the system can be configured to test a sample for presence of target nucleic acid sequences associated with the different sexual health conditions.

In some embodiments, the method includes collecting a sample, extracting or purifying a target nucleic acid from the sample, and amplifying the target nucleic acid sequences in order to detect the presence or absence of at least one sexual health condition. In some embodiments, the kit includes the amplification reagents to detect Chlamydia trachomatis, Neisseria gonorrhoeae, Trichomonas vaginalis, Treponema pallidum, Gardnerella vaginitis, human immunodeficiency virus, human papillomavirus infection, Hepatitis B, herpes simplex virus, Streptococcus pyogenes, or any subset of these. In some embodiments, the kit includes amplification reagents to detect Candida albicans, Candida dupliniensis, Candida parapsilosis, Candida tropicana, Candida Africana, Lactobacillus crispatus, Lactobacillus graseii, Lactobacillus inner, Lactobacillus jenseni, Gardnerella vaginalis, Apodium vaginae, Bactoroides fragilis, Mobiliscus curtiis, or any subset of these.

In some embodiments, amplifying the target nucleic acid sequences is performed using any suitable nucleic acid sequence amplification method known in the art. Non-limiting examples of nucleic acid sequence amplification methods include: polymerase chain reaction (PCR), reverse-transcriptase PCR (RT-PCR), real-time PCR, rolling circle amplification (RCA), ligase chain reaction (LCR), transcription-mediated amplification (TMA), and strand displacement amplification (SDA).

In some embodiments, the amplifying the target nucleic acid sequences is performed using real-time PCR. As used herein, “real-time PCR,” refers to a polymerase chain reaction (PCR) method in which the accumulation of amplification product is measured as the reaction progresses, in real time, with product quantification after each cycle. In some embodiments, the sample is extracted or purified to enrich the target DNA. Conventional PCR is an end point analysis where measurements of accumulated amplification product are after the desired amount of amplification is completed.

In some embodiments, real-time PCR is referred to as “quantitative PCR (qPCR)” as it enables quantitative detection of the target nucleic acid sequences. In some embodiments, the real-time detection of PCR amplification products includes the use of non-specific fluorescent dyes that intercalate with any double-stranded DNA. In some embodiments, the real-time detection of PCR amplification products includes the use of sequence-specific detectably-labeled DNA probes. Non-limiting examples real-time PCR techniques and systems are known in the art (see, e.g., Dorak, M. Tevfik, ed. Real-time PCR, Taylor & Francis (2007); and Fraga et al., “Real-time PCR,” Current protocols essential laboratory techniques: 10-3 (2008)) and are commercially available from a variety of sources (e.g., TAQMAN™ Real-Time PCR System (ThermoFisher Scientific, Waltham, MA)), any of which can be employed in the methods described herein.

Additional non-limiting examples of amplification and detection of target nucleic acid sequences that can be employed in the methods described herein include digital PCR (dPCR), digital droplet PCR (ddPCR), and Ncounter (Nanostring).

In some embodiments, sequence specific detectably-labeled DNA probe are hybridized to the target nucleic acid sequence, including the amplified target nucleic acid sequence. As used herein, the term “hybridization” refers to the formation of a duplex structure by two single-stranded nucleic acids due to complementary base pairing. Hybridization can occur between fully complementary nucleic acid strands or between “substantially complementary” nucleic acid strands that contain minor regions of mismatch.

### Kits for Detecting at Least One Sexual Health Condition

Provided herein is a kit for detecting status of a sexual health condition or for detecting whether at least one sexual health condition is present in a user. In some embodiments, the kit is an at-home kit for collecting a sample or can be used in a clinic (e.g., both the at-home kit or “collection kit” can be used interchangeably herein). In some embodiments, the kit is a combination of an at-home kit or collection kit for collecting a sample, and a laboratory kit for performing analysis on the sample. In some embodiments, the at-home kit or collection kit comprises the components of the laboratory kit. In some embodiments, the at home-kit and the laboratory kit comprise different components.

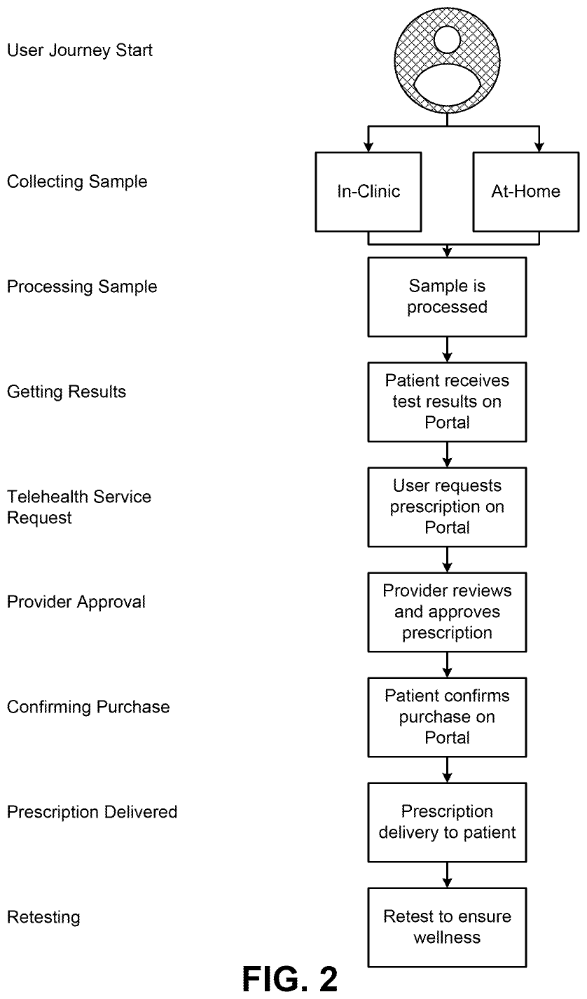

As shown in FIG. 2, the kits for detecting at least one sexual health condition can be used by a user at home or at a clinic. In a first step, the user receives and register's the collection kit, then uses the kit to collect one or more samples of interest. Next, the user sends the sample to a laboratory for further processing. The laboratory will process the sample to determine whether the user has one or more sexual health condition. Since the user has registered the kit with an online portal (e.g., using a mobile device, tablet, and the like), the user may receive a notification that the test results are available for viewing. The patient can then sign into an online portal to receive and view the test results that are displayed. If the patient tests positive for a sexual health condition, the user may schedule a telehealth appointment with a healthcare provider using the online portal. If approved by the healthcare provider, the healthcare provider can provide a prescription medication or suggest an over-the-counter medication to the user. The user can then confirm the purchase of the medication and initiate delivery of the medication to the user. At any time, the user can retest using a new or existing collection kit (e.g., if there are components in the kit that can be re-useable).

In some embodiments, the collection kit (e.g., used interchangeably herein as “at-home kit” or “collection-kit”) and/or laboratory kit comprises: (a) a buffer; (b) an amplification reaction mixture; and (c) primers, comprising at least one of: (i) a first pair of primers that bind specifically to a target nucleic acid sequence from Chlamydia trachomatis (e.g., any of the primers described herein that bind specifically to a target nucleic acid sequence from Chlamydia trachomatis), (ii) a second pair of primers that bind specifically to a target nucleic acid sequence from Neisseria gonorrhoeae (e.g., any of the primers described herein that bind specifically to a target nucleic acid sequence from Neisseria gonorrhoeae), and (iii) a third pair of primers that bind specifically to a target nucleic acid sequence from Trichomonas vaginalis (e.g., any of the primers described herein that bind specifically to a target nucleic acid sequence from Trichomonas vaginalis), or (iv) a combination thereof.

In some embodiments, the at-home kit and/or laboratory kit comprises primers that bind specifically to a target nucleic acid sequence selected from (e.g., forward and reverse primers described herein that bind specifically to a target nucleic acid sequence from): Candida albicans, Candida dupliniensis, Candida parapsilosis, Candida tropicana, Candida Africana, Lactobacillus crispatus, Lactobacillus graseii, Lactobacillus inner, Lactobacillus jenseni, Gardnerella vaginalis, Apodium vaginae, Bactoroides fragilis, Mobiliscus curtiis, or any subset of these.

In some embodiments, the at-home kit and/or laboratory kit comprises: a sample collection device for receiving the sample collected by a user at home or at a clinic; a storage container storing a preservation extraction, storage, or extraction buffer designed for receiving samples collected at home into the preservation buffer; a labeled shipping container for shipping the sample storage container holding the preservation buffer and the sample, the shipped preservation buffer and sample for analysis at a laboratory using: a DNA extraction kit; an amplification reaction mixture; and primers, comprising at least one of: a first pair of primers that bind specifically to a target nucleic acid sequence from Chlamydia trachomatis, a second pair of primers that bind specifically to a target nucleic acid sequence from Neisseria gonorrhoeae, and a third pair of primers that bind specifically to a target nucleic acid sequence from Trichomonas vaginalis, or a combination thereof.

In some embodiments, the collection kit comprises a sampling kit for collecting a sample for detection of a sexual health condition, comprising: a sample collection container for collecting a biological sample (e.g., a tube, or a swab); optionally a sample collection tool for transferring the biological sample from the sample collection container to a storage container; the storage container for receiving the biological sample, optionally a preservation buffer within the storage container; a sealable bag for placement of the storage container comprising the preservation buffer and the biological sample after a user has collected the biological sample. In some embodiments, the at-home kit further comprises a shipping label.

In some embodiments, the collection kit comprises a sampling kit for collecting a sample for detection of a sexual health condition, comprising: a sample collection container for collecting a biological sample (e.g., a tube, or a swab); a sample collection tool for transferring the biological sample from the sample collection container to a storage container; the storage container for receiving the biological sample, a preservation buffer within the storage container; a sealable bag for placement of the storage container comprising the preservation buffer and the biological sample after a user has collected the biological sample. In certain embodiments, when a swab is the sample collection device, no buffer or sample collection tool is needed. For example, if the sample is collected via a collection swab, the user or clinician can collect the sample from the user, and after collection, place the swab in a sample transport container for receiving the sample(s) collected.

In some embodiments, the at-home or collection kit further comprises an amplification reaction mixture; and primers, comprising at least one of: (i) a first pair of primers that bind specifically to a target nucleic acid sequence from Chlamydia trachomatis, (ii) a second pair of primers that bind specifically to a target nucleic acid sequence from Neisseria gonorrhoeae, and (iii) a third pair of primers that bind specifically to a target nucleic acid sequence from Trichomonas vaginalis, or (iv) a combination thereof.

In some embodiments, the at-home or collection kit comprises primers and optionally probes for specifically binding to at least one target selected from: Candida albicans, Candida dupliniensis, Candida parapsilosis, Candida tropicana, Candida Africana, Lactobacillus crispatus, Lactobacillus graseii, Lactobacillus inner, Lactobacillus jenseni, Gardnerella vaginalis, Apodium vaginae, Bactoroides fragilis, and Mobiliscus curtiis (see Table X).

In some embodiments, the at-home kit further comprises a transfer device for allowing the user to transfer the sample from the sample collection device to the storage container with the preservation buffer.

In some embodiments, the at-home kit further comprises a biohazard container for receiving and storing the sample storage container holding the preservation buffer and the sample, wherein the labeled shipping container is configured to hold the biohazard container with the sample storage container inside.

In some embodiments, the sample collection device is a collapsible cup. In some embodiments, the sample collection device is a tube. In some embodiments, the sample collection device carries an applicator (e.g., a swab). In some embodiments, the sample collection device is a swab collection device. In some embodiments, the applicator (e.g., sample collection tool) is a swab. In some embodiments, the swab is dry (e.g., does not contain any liquid within the swab). In some embodiments, the swab containers a dry swab until the applicator is modified to apply fluid to the swab. In some embodiments, the sample collection device is a saliva or oral fluid collection device.

In some embodiments, the at home kit further comprises a sample label for labeling the sample storage container with information about the user.

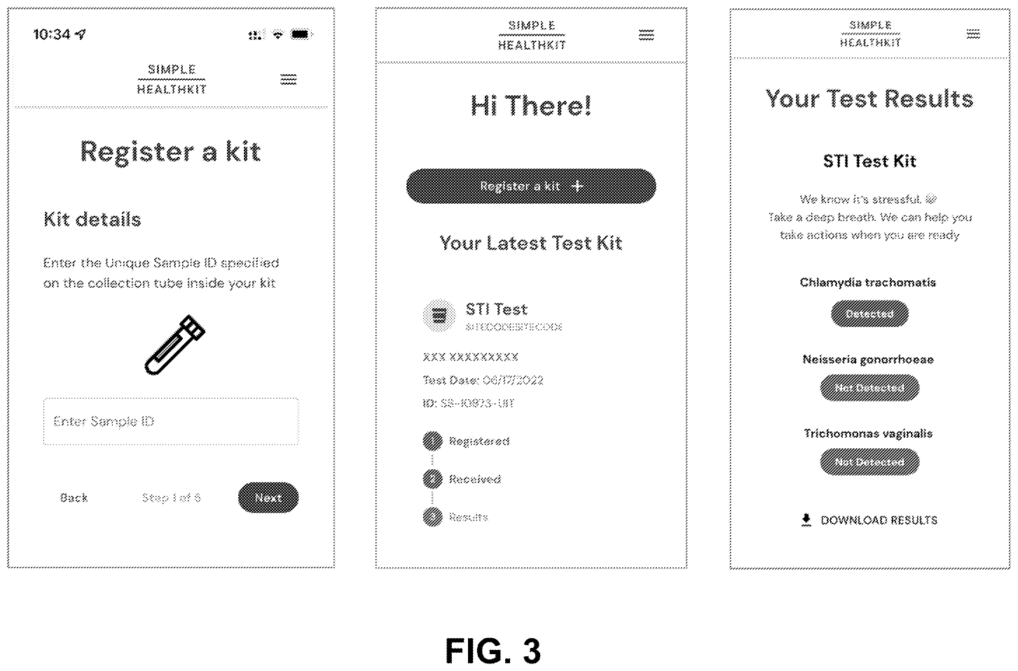

In some embodiments, the at-home kit comprises a code for scanning by a user's mobile device to register the kit (see e.g., FIG. 3 “Register a kit”). In some embodiments, the code for scanning by a user's mobile device to register the kit provides access to an online portal (FIG. 3). In some embodiments, the online portal provides a user interface between the user and a healthcare provider, and access to telemedicine with the healthcare provider. In some embodiments, the online portal displays an output result to the user as to the status of whether the user has a sexual health condition based on the analysis of the sample that the user has provided (FIG. 3 “Your Test Results”). In some embodiments, the access to telemedicine with the healthcare provider allows for communication with the healthcare provider. In some embodiments, the communication can be for purposes of running further tests, displaying results, communication results, providing telehealth visits, prescriptions and/or medications.

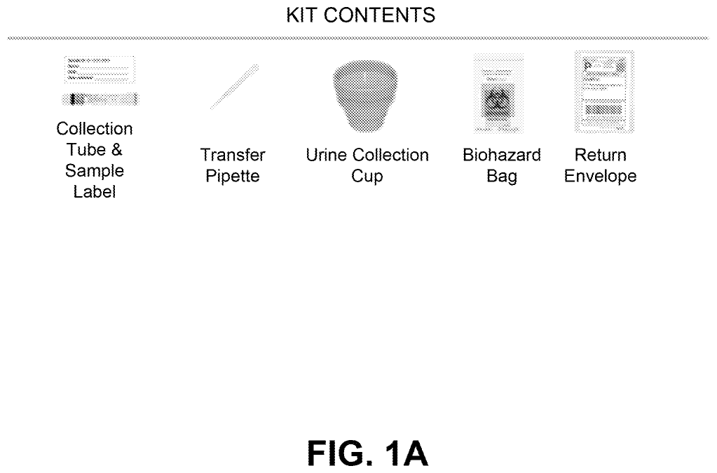

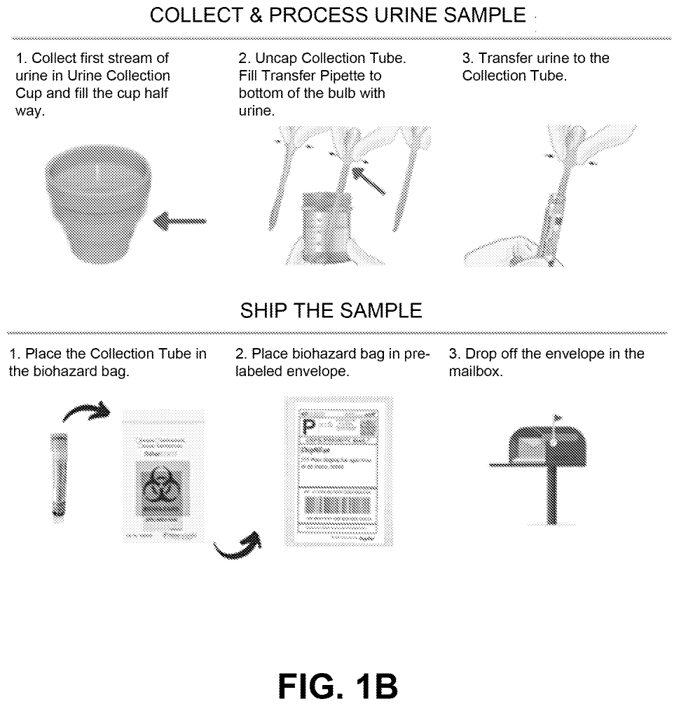

In some embodiments, the kit of the present disclosure also includes a pair of primers that bind specifically to a fourth target nucleic acid sequence from a control population of nucleic acid sequences (e.g., any of the primers described herein that bind specifically to a target nucleic acid sequence from a control population). In some embodiments, the kit also includes a sample collection tool (e.g., any of the sample collection tools described herein) and a transfer pipette. In some embodiments, the kit also includes a sample storage container (e.g., any of the sample storage containers described herein). In some embodiments, the kit also includes a collection tube. In some embodiments, a sample collection tool can be any tool suitable for the collection of a specimen that enables assessment of the presence or absence of a sexual health condition. In some embodiments, the sample collection tool is a transfer pipette. In some embodiments, the sample collection tool is a dropper. In some embodiments, the sample collection tool is a swab (e.g., dry swab). A non-limiting example of a collection container is shown in FIGS. 1A-1B.

Provided herein is an at-home and/or laboratory kit for detecting status of a sexual health condition, the kit comprising: (a) a sample collection kit (b) a buffer; (c) an amplification reaction mixture; and (d) primers, comprising at least one of: (i) a pair of primers that bind specifically to a target nucleic acid sequence from Chlamydia trachomatis (e.g., any of the primers described herein that bind specifically to a target nucleic acid sequence from Chlamydia trachomatis). In some embodiments, the kit also includes a pair of primers (e.g., any of the primers described in Table 3) that bind specifically to a nucleic acid sequence from a control population of nucleic acid sequences.

Provided herein is a kit for detecting status of a sexual health condition, the kit comprising: (a) a sample collection kit (b) a buffer; (c) an amplification reaction mixture; and (d) primers, comprising at least one of: (i) a pair of primers (e.g., any of the primers described herein that bind specifically to a target nucleic acid sequence from Neisseria gonorrhoeae) that bind specifically to a target nucleic acid sequence from Neisseria gonorrhoeae. In some embodiments, the kit also includes a pair of primers that bind specifically to a nucleic acid sequence from a control population of nucleic acid sequences (e.g., any of the primers described herein that bind specifically to a target nucleic acid sequence from a control population).

Provided herein is a kit for detecting status of a sexual health condition, the kit comprising: (a) a sample collection kit (b) a buffer; (c) an amplification reaction mixture; and (d) primers, comprising at least one of: (i) a pair of primers that bind specifically to a target nucleic acid sequence from Trichomonas vaginalis (e.g., any of the primers described herein that bind specifically to a target nucleic acid sequence from Trichomonas vaginalis). In some embodiments, the kit also includes a pair of primers that bind specifically to a nucleic acid sequence from a control population of nucleic acid sequences (e.g., any of the primers described herein that bind specifically to a target nucleic acid sequence from a control population).

### Extraction or Storage Buffer

In some embodiments, the method(s) and kit(s) provided herein include an extraction buffer. An extraction buffer allows the recovery of the target DNA from the organisms. In some embodiments, the extraction buffer is any buffer that enables extraction of nucleic acid (e.g., DNA) from a sample without compromising the integrity of the nucleic acid. In some embodiments, the extraction buffer includes phosphate buffered saline, Tris-buffered saline, HEPES buffered saline, a reducing agent, sodium hydroxide, Triton X-100, and octyl glucoside. In some embodiments, extraction buffer is provided in a commercial kit. Non-limiting examples of commercial kits that include an extraction buffer that can be employed in the methods described herein include: Qiagen DNeasy Miniprep Kit, Qiagen DNeasy Blood and Tissue Kit, Zymo Research DNA Miniprep Kit, Bio-Rad DNA Extraction Kits, Roche Cobas™ kit, among others. Non-limiting examples of extraction buffers are as described in PCT Publication No. WO 2022/031560, which is herein incorporated by reference in its entirety. In some embodiments, the extraction buffer includes a reducing agent. Non-limiting examples of reducing agents include: dithiothreitol (DTT), β-mercaptoethanol (β-ME), and tris(2-carboxyethyl)phosphine (TCEP).

In some embodiments, the storage or extraction buffer comprises one or more buffers selected from: sodium hydroxide, potassium hydroxide, sodium chloride, potassium chloride, sulfuric acid, nitric acid, hydrochloric acid, PBS (phosphate buffered saline), TBS (Tris buffered saline), tricine, HBS (HEPES buffered saline), 3-[(3-Cholamidopropyl) dimethylammonio]-1-propanesulfonate BugBuster™, octylthioglucoside, Triton X-100, octyl glucoside, BD™ MAX™ UVE Sample Buffer, potassium phosphate, ethylenediaminetetraacetic acid (EDTA), Tween™ 20 and ProClin™. In some embodiments, the extraction buffer comprises Zymo Research Quick-DNA™ Microprep Kit to extract DNA from the samples.

In some embodiments, the extraction or storage buffer is added directly to the sample collection tool or sample holder. In some embodiments, the sample is processed prior to being contacted with the extraction buffer. In some embodiments, the sample is added to the extraction buffer. In such cases, the method(s) and kit(s) can include two or more volumes of extraction buffer, two or more different extraction buffers, or a combination thereof.

In some embodiments, the buffer is only a storage buffer and not an extraction buffer. In some embodiments, the methods of the present disclosure do not include the step of extracting the nucleic acids from the sample. In such embodiments, the time for processing the sample is reduced by at least 1 hour, at least 1.5 hours, at least 2 hours, at least 3 hours, at least 4 hours, and the like. In such embodiments where extraction of the nucleic acid is not required, the sample is taken directly from the storage buffer and placed into the reaction container for the amplification step. In certain embodiments, the method does not include an extraction step, e.g., no extraction of the plurality of target nucleic acid is performed and the method is a direct to PCR method.

### Amplification Reaction (Polymerase and Buffers)

In some embodiments, the method(s) and kit(s) provided herein include an amplification reaction mixture. In some embodiments, the amplification reaction mixture includes a polymerase and a reaction buffer. In some embodiments, the amplification reaction mixture includes a polymerase comprising 5′ nuclease activity. Non-limiting examples of polymerase comprising a 5′ nuclease activity include: Taq DNA polymerase, DNA polymerase I, ϕ29 DNA polymerase, Bst DNA Polymerase, and the like

In some embodiments, the amplification reaction mixture includes: dNTPs, Magnesium (e.g., MgCl₂), buffer, and loading control dyes, or a combination thereof.

In some embodiments, the amplification reaction mixture includes dATP, dCTP, dGTP and dTTP. In some embodiments, the amplification reaction mixture replaces dTTP with dUTP.

In some embodiments, the amplification reaction mixture includes magnesium chloride (MgCl₂). In some embodiments, magnesium chloride is important for polymerase 5′ to 3′ exonuclease activity. In some embodiments, magnesium chloride concentrations for reactions containing dual-labeled probes is between 3-6 mM (e.g., any of the values of subranges therein).

In some embodiments, the amplification reaction mixture includes buffers. In some embodiments, the amplification reaction mixture includes stabilizers.

In some embodiments, the amplification reaction mixture comprises loading control dyes. A loading control dye is selected depending on the detection chemistry. In some embodiments, loading controls dyes include: SYBR Green I dye, ROX™, fluorescein and inert loading dyes. In some embodiments, a real-time PCR thermal cyclers requires a loading dye such as ROX to be included into each reaction to control for variability in the optical system and to normalize differences in signal intensity. In some embodiments, thermal cyclers require an initial fluorescein signal to create a virtual background when working with SYBR Green I dye assays (which have very low background). These may be supplied in the master mix or as separate components so that the appropriate concentration can be used.

In some embodiments, the amplification mixture includes non-specific fluorescent dyes that intercalate with any double-stranded DNA (dsDNA). The use of a dsDNA-binding dye, such as SYBR® Green I, represents an additional form of detection chemistry. When free in solution or with only single-stranded DNA (ssDNA) present, SYBR Green I dye emits light at low signal intensity. As the PCR progresses and the quantity of dsDNA increases, more dye binds to the amplicons and hence, the signal intensity increases. Non-limiting examples of non-specific fluorescent dyes that intercalate with any double-stranded DNA (dsDNA) include: EvaGreen dye, GelRed, GelGreen, SYBR Green I (U.S. Pat. Nos. 5,436,134 and 5,658,751), SYBR GreenEr, SYBR Gold, LC Green, LC Green Plus, BOXTO, BEBO, SYBR DX, SYTO9, SYTOX Blue, SYTOX Green, SYTOX Orange, SYTO dyes, POPO-1, POPO-3, BOBO-1, BOBO-3, YOYO-1, YOYO-3, TOTO-1, TOTO-3, PO-PRO-1, BO-PRO-1, YO-PRO-1, TO-PRO-1, JO-PRO-1, PO-PRO-3, LO-PRO-1, BO-PRO-3, YO-PRO-3, TO-PRO-3, TO-PRO-5, Ethidium Homodimer-1, Ethidium Homodimer-2, Ethidium Homodimer-3, propidium iodide, ethidium bromide, various Hoechst dyes, DAPI, ResoLight, Chromofy, and acridine homodimer.

In some embodiments, the amplification mixture includes one or more of: Oligonucleotide(s) (see Tables 1, 2, 3, and 4), RNase P (RP), IDT PrimeTime® Gene Expression Master Mix (2×), and PCR grade water.

In some embodiments, the method of the present disclosure comprises amplifying a target nucleic acid sequence of a plurality of target nucleic acid sequences by PCR Amplification using primers and an amplification reaction mixture. In certain embodiments, said amplifying comprises amplifying the primers and the amplification reaction mixture in 3 cycles using a thermocycler. In some embodiments, during a first cycle, the method comprises amplifying the sample containing the target nucleic acids, primers and amplification mixture in a first cycle at a temperature ranging from 20° C. to 80° C. (e.g., 20° C., 30° C., 40° C., 47° C., 48° C., 49° C., 50° C., 51° C., 52° C., 53° C., 55° C., 56° C., 57° C., 60° C., 70° C., or 80° C.) and for a time period ranging from 30 seconds to 5 minutes (e.g., 30 seconds, 1 minute, 2 minutes, 3 minutes, 4 minutes, or 5 minutes). In some embodiments, during a first stage, the method comprises amplifying the sample containing the target nucleic acids, primers and amplification mixture in a second stage at a temperature ranging from 60° C. to 130° C. (e.g., 60° C., 70° C., 80° C., 85° C., 90° C., 91° C., 92° C., 93° C., 94° C., 95° C., 96° C., 97° C., 98° C., 99° C., 100° C., 105° C., 110° C., 120° C., or 130° C.) and for a time period ranging from 1 minute to 8 minutes (e.g., 1 minute, 2 minutes, 3 minutes, 4 minutes, 5 minutes, 6 minutes, 7 minutes, or 8 minutes). In some embodiments, during a third stage, the method comprises amplifying the sample containing the target nucleic acids, primers and amplification mixture in a third stage at a temperature ranging from 30° C. to 130° C. (e.g., 30° C., 40° C., 45° C., 46° C., 47° C., 48° C., 49° C., 50° C., 51° C., 52° C., 53° C., 55° C., 56° C., 57° C., 58° C., 59° C., 60° C., 61° C., 62° C., 63° C., 64° C., 65° C., 66° C., 70° C., 72° C., 80° C., 82° C., 90° C., 91° C., 92° C., 93° C., 94° C., 95° C., 96° C., 97° C., 98° C., 99° C., 100° C., 110° C., 120° C., or 130° C.) and for a time period ranging from 1 second to 2 minutes (1 second, 10 seconds, 15 seconds, 30 seconds, 1 minute, 1.5 minutes or 2 minutes). Table 1 provides exemplary the amplification profile (e.g., cycles, time, temperature, and stage) for analyzing the sample using PCR.

TABLE 1
Amplification profile

| Stage | Temperature (° C.) | Time | Cycle |
|---|---|---|---|
| 1 | 47-53 | 1-2 m | 1 |
| 2 | 92-98 | 3-8 m | 1 |
| 3 | 92-98 | 8-23 s | 45 |
| | 59.5-65.5 | 15-45 s | |

### Probes

In some embodiments, the method(s) and kit(s) include a first pair of primers, a second pair of primers, a third pair of primers, a fourth pair of primers, or a combination thereof where each pair of primers optionally include a probe, wherein the probe binds specifically to the respective target nucleic acid sequence. In some embodiments, a probe is selected from: a dual-labeled probe, a molecular beacon, a LightCycler® probe, and a Scorpions® probe. Concentration ranges of primers, probes, amplification reaction mixture, and DNA template is provided in Table 2 below.

TABLE 2
Concentration ranges of CT, NG, and TV primer pairs, probes,
amplification reaction mixture, and sample for amplification

| | |
|---|---|
| Primers/Probe | |
| CT-Forward | 0.05-1 uM |
| CT- Reverse | 0.05-1 uM |
| CT-Probe | 0.05-1 uM |
| NG- Forward | 0.05-1 uM |
| NG- Reverse | 0.05-1 uM |
| NG-Probe | 0.05-1 uM |
| TV- Forward | 0.05-1 uM |
| TV-Reverse | 0.05-1 uM |
| TV-Probe | 0.05-1 uM |
| 2 x PCR mastermix | 5-10 ul |
| DNA template | 1-10 ul |
| total sample volume | 10-20 ul |

In some embodiments, a probe is a dual-labeled probe. In some embodiments, the dual-labeled probe includes on the 5′ end: a fluorescent reporter dye; on the 3′ end: nonfluorescent quencher (NFQ); and also on the 3′ end (attached to the NFQ): minor groove binder (MGB). In some embodiments, the 5′ fluorescent reporter dye is detected by a qPCR instrument (e.g., QuantStudio3) in order to quantitate the amount of PCR amplification product in the sample. When the polymerase reaches a probe comprising the 5′ fluorescent reporter dye, the polymerases 5′ nuclease activity cleaves the probe, separating the dye from the quencher. With each PCR cycle, more dye molecules are released, resulting in an increase in fluorescence intensity proportional to the amount of amplicon synthesized. The 3′ NFQ quenches the 5′ fluorescent reporter dye until separated from it by the exonuclease activity of the polymerase that comprises 5′ nuclease activity. In some embodiments, using an NFQ instead of a fluorescent quencher increases the sensitivity of an assay by providing a greater signal-to-noise ratio.

In some embodiments, the NFQ on the probes minimizes background, maximizes amplification efficiency, and allows for high sensitivity and accuracy to reliably detect targets present at 10 or fewer copies.

In some embodiments, the MGB is a small molecule that fits into the minor groove of double-stranded DNA. The MGB increases the melting temperature € without increasing probe length; therefore, probes can be designed for difficult sequences (e.g., AT-rich sequences), making it easier to design probes over exon-exon boundaries.

Non-limiting examples of a fluorescent reporter dye include: Fluorescein, 6-FAM™, HEX™, TET™, TAMRA™, JOE™, ROX™, Cyanine 3, Cyanine 5, Cyanine 5.5, Texas Red®, Rhodamine, Rhodamine Green™, Rhodamine Red™, Oregon Green® 488, Oregon Green 500, and Oregon Green 514.

Non-limiting examples of a quencher include: DABCYL dT, BHQ®-1, BHQ-2, BHQ-3, TAMRA, ZEN/IBFQ=ZEN-Iowa Black FQ, and IBFQ=Iowa Black FQ.

In some embodiments, a probe includes one or more modified nucleotides. For example, a locked nucleic acid (LNA) can be incorporated into any of our qPCR probe types, providing enhanced sensitivity and specificity for your assay. LNA is a novel type of nucleic acid analog containing a 2′-O, 4′-C methylene bridge. When used with any standard bases (A,C,G,T, or U), probes synthesized using LNA have greater thermal stability than conventional DNA or RNA and therefore form a stronger bond with the complementary sequence. The introduction of LNA chemistry into a qPCR probe may result in an increase in the TM of up to 8° C. per LNA monomer substitution in medium salt conditions compared to a DNA fluorescent probe. In some embodiments, a probe includes an optimized TM level and hybridization specificity as a result of specific placement of the LNA base(s) in the probe design.

In some embodiments, the amplification products are measured as they are produced using a fluorescent label. In some embodiments, during amplification, a fluorescent dye binds, either directly or indirectly via a labeled hybridizing probe, to the accumulating DNA molecules, and fluorescence values are recorded during each cycle of the amplification process. In some embodiments, the fluorescence signal is directly proportional to DNA concentration over a broad range, and the linear correlation between PCR product and fluorescence intensity is used to calculate the amount of template present at the beginning of the reaction. The point at which fluorescence is first detected as statistically significant above the baseline or background, is called the threshold cycle or Ct Value. In some embodiments, this threshold is established to quantify the amount of DNA in the samples. It is inversely correlated to the logarithm of the initial copy number. The threshold should be set above the amplification baseline and within the exponential increase phase. In some embodiments, the PCR instruments automatically calculates the threshold level of fluorescence signal by determining the baseline (background) average signal and setting a threshold 10-fold higher than this average.

Primers and Probes for Chlamydia trachomatis (CT)

In some embodiments, the kits and methods include a pair of primers that bind specifically to a target nucleic acid sequence from Chlamydia trachomatis. In some embodiment, the pair of primers that bind specifically to a target nucleic acid sequence from Chlamydia trachomatis include: a pair of primers comprise a forward primer having a nucleotide sequence selected from SEQ iD NOs: 1-19, a reverse primer having a nucleotide sequence selected from SEQ iD NOs: 20-35, and optionally, a probe having a nucleotide sequence selected from SEQ iD NOs: 36-47. Non-limiting examples of primers that bind specifically to a target nucleic acid sequence from Chlamydia trachomatis include the primers in Table 3.

TABLE 3
Primers for Chlamydia trachomatis

| | | SEQ | |
|---|---|---|---|
| Target | Primer | ID | Sequence 5′ - 3′ |
| Chlamydia | Forward | 1 | TTTACTATTAATAAACCTAAAGGGTATG |
| trachomatis | | | T |
| | | 2 | GGTATGTAGGYAAGGAGTTTCCTC |
| | | 3 | GATATGTCGGGGCGGAATTTCCAC |
| | | 4 | GACACCAAAGCGAAAGACAACAC |
| | | 5 | GATACGTTGGACAAGAATTCCCTC |
| | | 6 | GAAGCCTGAGAAGGTTTCTGACTAGG |
| | | 7 | GTTGCCAACGCCATTTACGATGGA |
| | | 8 | GCTGATTACGAGCCGATAAGAACTACTG |
| | | 9 | TTTACTATTAATAAGCCGAAAGGATATG |
| | | | T |
| | | 10 | TAGGAGCTTCATTCCAATATGCTCA |
| | | 11 | GCTTTCTTTCGAACGAGGGTTGA |
| | | 12 | GATATGTAGGGCAAGAATTCCCTC |
| | | 13 | AGACTCATGAACCGGAGCAAC |
| | | 14 | CCAACGCCATTTACGATGGA |
| | | 15 | GATATGTAGGYAAGGAGTTTCCTC |
| | | 16 | TTTACTATCAATAAGCCTAAAGGATATG |
| | | | T |
| | | 17 | CACCTGACACCATTCCGAACTCAG |
| | | 18 | GGAAGAAGACCTGGTCAATCTATTA |
| | | 19 | GATATGTAGGCAAGGAGTTTCCTC |
| Chlamydia | Reverse | 20 | ACCAAAGCGAAAGACAACACTTC |
| [symbol] | | | |
| | | 21 | ACCATTTAACTCCAATGTARGGAGTGAA |
| | | | CAT |
| | | 22 | GTAGGGAGTGAACATATTCAGTCTGTAA |
| | | 23 | GCTGCTGCACCACCACTCGTATTTTCTG |
| | | 24 | CAGCTCCAGCAATAGTTGGGTTAAGC |
| | | 25 | GATAGCTGCTAATGCATGGTAATG |
| | | 26 | ACTCATGAACCGGAGCAACCT |
| | | 27 | GCTTCCAGCCTCTCAGCCTTCCACTC |
| | | 28 | GTAAGGAGTGAACATATTTAATCTGTAA |
| | | 29 | TCTTCTCAGTCTCTTGGATAATG |
| | | 30 | GTAGGGAGTGAACATATTGAGTCTGTAA |
| | | 31 | GTCTCGTCCTCACCGCGCTACTCTTAAC |
| | | 32 | GTAAGGAGTGAACATATTCAGTCTGTAA |
| | | 33 | GTAGGGAGTGAACATATTCAATCTGTAA |
| | | 34 | TGGCGACGACCTACTCTCCCATAC |
| | | 35 | GTCTTCTCAGTCTCTTGGATAATGG |

[symbol]

indicates data missing or illegible when filed

In some embodiments, the concentration of a forward primer having a nucleotide sequence selected from SEQ iD NOs: 1-19 ranges from 0.05 uM to 1 uM. In some embodiments, the concentration of the forward primer is 0.5 uM. 0.6 uM, 0.7 uM, 0.8 uM, 0.9 uM, or 1 uM. In some embodiments, the concentration of a reverse primer having a nucleotide sequence selected from SEQ iD NOs: 20-35 ranges from 0.05 uM to 1 uM. In some embodiments, the concentration of the reverse primer is 0.5 uM, 0.6 uM, 0.7 uM, 0.8 uM, 0.9 uM, or 1 uM.

Non-limiting examples of probes that bind specifically to a target nucleic acid sequence from Chlamydia trachomatis include the probes in Table 4. Non-limiting examples of probes that bind specifically to a target nucleic acid sequence from Chlamydia trachomatis include the probes in Table 4. In some embodiments, the concentration of a probe having a nucleotide sequence selected from SEQ iD NOs: 36-47 ranges from 0.05 uM to 1 uM. In some embodiments, the concentration of the probe is 0.5 uM. 0.6 uM, 0.7 uM, 0.8 uM, 0.9 uM, or 1 uM.

TABLE 4
Probes for Chlamydia trachomatis

| | | SEQ | |
|---|---|---|---|
| | | ID | |
| | | NO: | Sequence 5′ - 3′ |
| Chlamydia | Probe | 36 | ACCACAGCAAAGAGACTCCCGTA |
| [symbol] | | | |
| | | 37 | TTGAYTACCATGARTGGCAAG |
| | | 38 | TTACCATGARTGGCAAGCAAGTTTA |
| | | 39 | AAGCCTCTGATCGCTGATGGTACTAT |
| | | 40 | CTTGCTTGCCACTCATGGTAATCGA |
| | | 41 | GGCAAGCGTTTATATGTAAGAGCAAGCATTC |
| | | 42 | TGCCTTAGGAGCCCATCCTGTACATGCA |
| | | 43 | AGAGTATTGGTGGCTCTAGAACAAGTG |
| | | 44 | TGCCTTAGGAGCCCATCCTGTACAT |
| | | 45 | TGGGATGGTAGAAGTTATAGGTCTTGA |
| | | 46 | GAAGGCCTCTTACGTGGATGAGAGGGC |
| | | 47 | CCACAGCAAAGAGACTCCCGTAGACCG |

[symbol]

indicates data missing or illegible when filed

Primers and Probes for Neisseria gonorrhoeae (NG)

In some embodiments, the kits and methods include a pair of primers that bind specifically to a target nucleic acid sequence from Neisseria gonorrhoeae. Non-limiting examples of probes that bind specifically to a target nucleic acid sequence from Neisseria gonorrhoeae include the probes in Table 5. In some embodiment, the pair of primers that bind specifically to a target nucleic acid sequence from Neisseria gonorrhoeae include: a pair of primers comprise a forward primer having a nucleotide sequence selected from SEQ ID NO: 48-59, a reverse primer having a nucleotide sequence selected from SEQ ID NO: 60-70, and optionally, a probe having a nucleotide sequence selected from SEQ ID NO: 71-84. In some embodiment, the pair of primers that bind specifically to a target nucleic acid sequence from Neisseria gonorrhoeae include: a pair of primers comprise a forward primer and a reverse primer as described in U.S. Application Publication No.: US2008003591, which is hereby incorporated by reference in its entirety. Additional pairs of primers that bind specifically to a target nucleic acid sequence from Neisseria gonorrhoeae, can be found in Perera et al., 2017 (J Clin Microbiol. 2017 November; 55(11): 3201-3209). In some embodiments, the concentration of a forward primer having a nucleotide sequence selected from SEQ iD NOs: 48-59 ranges from 0.05 uM to 1 uM. In some embodiments, the concentration of the forward primer is 0.5 uM. 0.6 uM, 0.7 uM, 0.8 uM, 0.9 uM, or 1 uM. In some embodiments, the concentration of a reverse primer having a nucleotide sequence selected from SEQ iD NOs: 60-70 ranges from 0.05 uM to 1 uM. In some embodiments, the concentration of the reverse primer is 0.5 uM. 0.6 uM, 0.7 uM, 0.8 uM, 0.9 uM, or 1 uM. In some embodiments, the concentration of a probe having a nucleotide sequence selected from SEQ ID NOs: 71-84 ranges from 0.05 uM to 1 uM. In some embodiments, the concentration of the probe is 0.5 uM. 0.6 uM, 0.7 uM, 0.8 uM, 0.9 uM, or 1 uM. Non-limiting examples of primers and probes that bind specifically to a target nucleic acid sequence from Neisseria gonorrhoeae include the probes in Table 5.

TABLE 5
Primers and Probes for Neisseria gonorrhoeae

| | | SEQ | |
|---|---|---|---|
| | Primer/ | ID | |
| Target | Probe | NO: | Sequence 5′ - 3′ |
| Neisseria | Forward | 48 | GCTACGCATACCCGCGTTGC |
| gonorrhoeae | | | |
| | | 49 | GCGTGTCTGAAGAAGGC |
| | | 50 | GTCACGCATACCCGCGTTGC |
| | | 51 | TACCACCCCCACGGCGATTT |
| | | 52 | TATCGGAACGTACCGGGTAGC |
| | | 53 | GTTGAAACACCGCCCGG |
| | | 54 | ACTGTACGCGATGCACGAGC |
| | | 55 | GCGATTCCGCAGTTTACGA |
| | | 56 | CAGCATTCAATTTGTTCCGAGTC |
| | | 57 | GTTGGATGCCCGTACTGGG |
| | | 58 | TATCGGAACGTACCGGGTAG |
| | | 59 | TATCGGAACGTACCGGGTAGC |
| Neisseria | Reverse | 60 | CGAAGACCTTCGAGCAGACA |
| gonorrhoeae | | | |
| | | 61 | GCTTATTCTTCAGGTACCGTCAT |
| | | 62 | GGGATTTCACATCCTGCT |
| | | 63 | GAACTGGTTTCATCTGATTACTTTCC |
| | | | A |
| | | 64 | ACCCACGCTTTCGGAC |
| | | 65 | GTATTACCGCGGCTGCTGGCA |
| | | 66 | ACCGATTTTGTAAGGCAGGG |
| | | 67 | CGAAGACCTTCGAGCAGACA |
| | | 68 | TCGGTTTTGGGTTCGTCCAAA |
| | | 69 | CGAAATTTTGCGCCATACGGACGAT |
| | | 70 | CGGTTTGACCGGTTAAAAAAAGAT |
| Neisseria | Probe | 71 | AGCCCTGCTATGACTATCAACCCTGC |
| gonorrhoeae | | | C |
| | | 72 | CTTTGAACCATCAGTGAAA |
| | | 73 | GGCCGCCGATATTGGCAACA |
| | | 74 | CGGGTTGTAAAGGACTTTTGTCAGGG |
| | | | AA |
| | | 75 | AAGGCTGTTGCCAATATCGGCGG |
| | | 76 | GCCAATATCGGCGGCC |
| | | 77 | AATACTGCCTTGCTGATGCGTAACTG |
| | | | CCGG |
| | | 78 | TTGTCAGGGAAGAAAAGGCTGT |
| | | 79 | CCCTTCAACATCAGTGAAA |
| | | 80 | CGTTCTTGACGCTCCATATCGCTATG |
| | | | AA |
| | | 81 | GGCCGCCGATATTGGCAA |
| | | 82 | CGGCAAAGTGGATGCAACCGA |
| | | 83 | CGCCTATACGCCTGCTACTTTCACGC |
| | | 84 | GCCTTTTCTTCCCTGACAAAAGTCCT |

Primers and Probes for Trichomonas vaginalis (TV)

In some embodiments, the kits and methods include a pair of primers that bind specifically to a target nucleic acid sequence from Trichomonas vaginalis. In some embodiment, the pair of primers that bind specifically to a target nucleic acid sequence from Trichomonas vaginalis include: a pair of primers having a forward primer having a nucleotide sequence selected from SEQ iD NOs: 85-95, a reverse primer having a nucleotide sequence selected from SEQ iD NOs: 96-104, and optionally, a probe having a nucleotide sequence selected from SEQ iD NOs: 105-115. In some embodiments, the concentration of a forward primer having a nucleotide sequence selected from SEQ iD NOs: 85-95 ranges from 0.05 uM to 1 uM. In some embodiments, the concentration of the forward primer is 0.5 uM. 0.6 uM, 0.7 uM, 0.8 uM, 0.9 uM, or 1 uM. In some embodiments, the concentration of a reverse primer having a nucleotide sequence selected from SEQ iD NOs: 96-104 ranges from 0.05 uM to 1 uM. In some embodiments, the concentration of the reverse primer is 0.5 uM. 0.6 uM, 0.7 uM, 0.8 uM, 0.9 uM, or 1 uM. In some embodiments, the concentration of a probe having a nucleotide sequence selected from SEQ iD NOs: 105-115 ranges from 0.05 uM to 1 uM. In some embodiments, the concentration of the probe is 0.5 uM. 0.6 uM, 0.7 uM, 0.8 uM, 0.9 uM, or 1 uM. Non-limiting examples of primers and probes that bind specifically to a target nucleic acid sequence from Trichomonas vaginalis include the probes in Table 6.

TABLE 6
Primer and Probes Trichomonas vaginalis

| | | SEQ | |
|---|---|---|---|
| | Primer/ | ID | |
| Target | Probe | NO: | Sequence 5′ - 3′ |
| Trichomonas | Forward | 85 | CACTCAAGCTCACAACACCAACA |
| vaginalis | | | |
| | | 86 | CATTGATAACGAAGCTCTTTACGAT |
| | | 87 | CCAGAAGTGGGCTACACACC |
| | | 88 | CWCTTTACGATATYTGCTTCCGTACA |
| | | 89 | AACATCCCCAACATCTT |
| | | 90 | AAAGATGGGTGTTTTAAGCTAGATAA |
| | | | GG |
| | | 91 | GATCATGTTCTATCTTTTCA |
| | | 92 | AAGATGGGTGTTTTAAGCTAGATAAG |
| | | | GT |
| | | 93 | CATTGACCACACGGACAAAAAG |
| | | 94 | TAATGGCAGAATCTTTGGAG |
| | | 95 | ATTGTCGAACATTGGTCTTACCCTC |
| Trichomonas | Reverse | 96 | GCATGTTGTGCCGGACATAACCAT |
| [symbol] | | | |
| | | 97 | ATACCAAGGCCGGAAGCAC |
| | | 98 | CCATTCTTTTAGACCCTT |
| | | 99 | CGTCTTCAAGTATGCCCCAGTAC |
| | | 100 | GAACTTTAACCGAAGGACTTC |
| | | 101 | TCTGTGCCGTCTTCAAGTATGC |
| | | 102 | TGCCGGACATAACCATGGA |
| | | 103 | GATCACCACCTTAGTTTACA |
| | | 104 | CGAAGTGCTCGAATGCGA |
| Trichomonas | Probe | 105 | GACCTCTAGAAGAAGACTCAG |
| vaginalis | | | |
| | | 106 | CCGAAGTTCATGTCCTCTCCAAGCG |
| | | 107 | CCGAAGTTCATGTCCTCTCCAAGCGT |
| | | 108 | ACGGCGATCTTAACCACCTTGTTTC |
| | | 109 | ACGGCGATCTTAAC |
| | | 110 | AGTTCATGTCCTCTCCAAGCGTAAGT |
| | | 111 | GCTAAACTCGATCTCGGTCGAGAAGC |
| | | 112 | AAGCTCACAACACCAACATACGGCGA |
| | | 113 | TCTGGCAAGATCAAGGACATCCTCC |
| | | 114 | GAGTTAGGGTCTAATGTTTGATGTG |
| | | 115 | TCATTTCGGATGGTCAAGCAGCCA |

[symbol]

indicates data missing or illegible when filed

### Primers and Probes for Additional Targets

In some embodiments, the kits and methods include a pair of primers that bind specifically to a target nucleic acid sequence from Candida albicans. In some embodiment, the pair of primers that bind specifically to a target nucleic acid sequence from Candida albicans include: a pair of primers having a forward primer having a nucleotide sequence of SEQ ID NO: 116, a reverse primer having a nucleotide sequence of SEQ ID NO: 117, and optionally, a probe having a nucleotide sequence of SEQ ID NO: 118.

In some embodiments, the concentration of a forward primer having a nucleotide sequence of SEQ ID NO: 116 ranges from 0.05 uM to 1 uM. In some embodiments, the concentration of the forward primer is 0.5 uM. 0.6 uM, 0.7 uM, 0.8 uM, 0.9 uM, or 1 uM. In some embodiments, the concentration of a reverse primer having a nucleotide sequence of SEQ ID NO: 117 ranges from 0.05 uM to 1 uM. In some embodiments, the concentration of the reverse primer is 0.5 uM. 0.6 uM, 0.7 uM, 0.8 uM, 0.9 uM, or 1 uM. In some embodiments, the concentration of a probe having a nucleotide sequence of SEQ ID NO: 118 ranges from 0.05 uM to 1 uM. In some embodiments, the concentration of the probe is 0.5 uM. 0.6 uM, 0.7 uM, 0.8 uM, 0.9 uM, or 1 uM. Non-limiting examples of primers and probes that bind specifically to a target nucleic acid sequence from Candida albicans include the probes in Table X.

In some embodiments, the kits and methods include a pair of primers that bind specifically to a target nucleic acid sequence from Candida dupliniensis. In some embodiment, the pair of primers that bind specifically to a target nucleic acid sequence from Candida dupliniensis include: a pair of primers having a forward primer having a nucleotide sequence of SEQ ID NO: 119, a reverse primer having a nucleotide sequence of SEQ ID NO: 120, and optionally, a probe having a nucleotide sequence of SEQ ID NO: 121.

In some embodiments, the concentration of a forward primer having a nucleotide sequence of SEQ ID NO: 119 ranges from 0.05 uM to 1 uM. In some embodiments, the concentration of the forward primer is 0.5 uM. 0.6 uM, 0.7 uM, 0.8 uM, 0.9 uM, or 1 uM. In some embodiments, the concentration of a reverse primer having a nucleotide sequence of SEQ ID NO: 120 ranges from 0.05 uM to 1 uM. In some embodiments, the concentration of the reverse primer is 0.5 uM. 0.6 uM, 0.7 uM, 0.8 uM, 0.9 uM, or 1 uM. In some embodiments, the concentration of a probe having a nucleotide sequence of SEQ ID NO: 121 ranges from 0.05 uM to 1 uM. In some embodiments, the concentration of the probe is 0.5 uM. 0.6 uM, 0.7 uM, 0.8 uM, 0.9 uM, or 1 uM. Non-limiting examples of primers and probes that bind specifically to a target nucleic acid sequence from Candida dupliniensis include the probes in Table X.

In some embodiments, the kits and methods include a pair of primers that bind specifically to a target nucleic acid sequence from Candida parapsilosis. In some embodiment, the pair of primers that bind specifically to a target nucleic acid sequence from Candida parapsilosis include: a pair of primers having a forward primer having a nucleotide sequence of SEQ ID NO: 122, a reverse primer having a nucleotide sequence of SEQ ID NO: 123, and optionally, a probe having a nucleotide sequence of SEQ ID NO: 124.

In some embodiments, the concentration of a forward primer having a nucleotide sequence of SEQ ID NO: 122 ranges from 0.05 uM to 1 uM. In some embodiments, the concentration of the forward primer is 0.5 uM. 0.6 uM, 0.7 uM, 0.8 uM, 0.9 uM, or 1 uM. In some embodiments, the concentration of a reverse primer having a nucleotide sequence of SEQ ID NO: 123 ranges from 0.05 uM to 1 uM. In some embodiments, the concentration of the reverse primer is 0.5 uM. 0.6 uM, 0.7 uM, 0.8 uM, 0.9 uM, or 1 uM. In some embodiments, the concentration of a probe having a nucleotide sequence of SEQ ID NO: 124 ranges from 0.05 uM to 1 uM. In some embodiments, the concentration of the probe is 0.5 uM. 0.6 uM, 0.7 uM, 0.8 uM, 0.9 uM, or 1 uM. Non-limiting examples of primers and probes that bind specifically to a target nucleic acid sequence from Candida parapsilosis include the probes in Table X.

In some embodiments, the kits and methods include a pair of primers that bind specifically to a target nucleic acid sequence from Candida tropicana. In some embodiment, the pair of primers that bind specifically to a target nucleic acid sequence from Candida tropicana include: a pair of primers having a forward primer having a nucleotide sequence of SEQ ID NO: 125, a reverse primer having a nucleotide sequence of SEQ ID NO: 126, and optionally, a probe having a nucleotide sequence of SEQ ID NO: 127.

In some embodiments, the concentration of a forward primer having a nucleotide sequence of SEQ ID NO: 125 ranges from 0.05 uM to 1 uM. In some embodiments, the concentration of the forward primer is 0.5 uM. 0.6 uM, 0.7 uM, 0.8 uM, 0.9 uM, or 1 uM. In some embodiments, the concentration of a reverse primer having a nucleotide sequence of SEQ ID NO: 126 ranges from 0.05 uM to 1 uM. In some embodiments, the concentration of the reverse primer is 0.5 uM. 0.6 uM, 0.7 uM, 0.8 uM, 0.9 uM, or 1 uM. In some embodiments, the concentration of a probe having a nucleotide sequence of SEQ ID NO: 127 ranges from 0.05 uM to 1 uM. In some embodiments, the concentration of the probe is 0.5 uM. 0.6 uM, 0.7 uM, 0.8 uM, 0.9 uM, or 1 uM. Non-limiting examples of primers and probes that bind specifically to a target nucleic acid sequence from Candida tropicana include the probes in Table X.

In some embodiments, the kits and methods include a pair of primers that bind specifically to a target nucleic acid sequence from cand-35-erifiedcana. In some embodiment, the pair of primers that bind specifically to a target nucleic acid sequence from cand-35-erifiedcana include: a pair of primers having a forward primer having a nucleotide sequence of SEQ ID NO: 128, a reverse primer having a nucleotide sequence of SEQ ID NO: 129, and optionally, a probe having a nucleotide sequence of SEQ ID NO: 130.

In some embodiments, the concentration of a forward primer having a nucleotide sequence of SEQ ID NO: 128 ranges from 0.05 uM to 1 uM. In some embodiments, the concentration of the forward primer is 0.5 uM. 0.6 uM, 0.7 uM, 0.8 uM, 0.9 uM, or 1 uM. In some embodiments, the concentration of a reverse primer having a nucleotide sequence of SEQ ID NO: 129 ranges from 0.05 uM to 1 uM. In some embodiments, the concentration of the reverse primer is 0.5 uM. 0.6 uM, 0.7 uM, 0.8 uM, 0.9 uM, or 1 uM. In some embodiments, the concentration of a probe having a nucleotide sequence of SEQ ID NO: 130 ranges from 0.05 uM to 1 uM. In some embodiments, the concentration of the probe is 0.5 uM. 0.6 uM, 0.7 uM, 0.8 uM, 0.9 uM, or 1 uM. Non-limiting examples of primers and probes that bind specifically to a target nucleic acid sequence from cand-35-erifiedcana include the probes in Table X.

In some embodiments, the kits and methods include a pair of primers that bind specifically to a target nucleic acid sequence from Lactobacillus crispatus. In some embodiment, the pair of primers that bind specifically to a target nucleic acid sequence from Lactobacillus crispatus include: a pair of primers having a forward primer having a nucleotide sequence of SEQ ID NO: 131, a reverse primer having a nucleotide sequence of SEQ ID NO: 132, and optionally, a probe having a nucleotide sequence of SEQ ID NO: 133.

In some embodiments, the concentration of a forward primer having a nucleotide sequence of SEQ ID NO: 131 ranges from 0.05 uM to 1 uM. In some embodiments, the concentration of the forward primer is 0.5 uM. 0.6 uM, 0.7 uM, 0.8 uM, 0.9 uM, or 1 uM. In some embodiments, the concentration of a reverse primer having a nucleotide sequence of SEQ ID NO: 132 ranges from 0.05 uM to 1 uM. In some embodiments, the concentration of the reverse primer is 0.5 uM. 0.6 uM, 0.7 uM, 0.8 uM, 0.9 uM, or 1 uM. In some embodiments, the concentration of a probe having a nucleotide sequence of SEQ ID NO: 133 ranges from 0.05 uM to 1 uM. In some embodiments, the concentration of the probe is 0.5 uM. 0.6 uM, 0.7 uM, 0.8 uM, 0.9 uM, or 1 uM. Non-limiting examples of primers and probes that bind specifically to a target nucleic acid sequence from Lactobacillus crispatus include the probes in Table X.

In some embodiments, the kits and methods include a pair of primers that bind specifically to a target nucleic acid sequence from Lactobacillus graseii. In some embodiment, the pair of primers that bind specifically to a target nucleic acid sequence from Lactobacillus graseii include: a pair of primers having a forward primer having a nucleotide sequence of SEQ ID NO: 134, a reverse primer having a nucleotide sequence of SEQ ID NO: 135, and optionally, a probe having a nucleotide sequence of SEQ ID NO: 136.

In some embodiments, the concentration of a forward primer having a nucleotide sequence of SEQ ID NO: 134 ranges from 0.05 uM to 1 uM. In some embodiments, the concentration of the forward primer is 0.5 uM. 0.6 uM, 0.7 uM, 0.8 uM, 0.9 uM, or 1 uM. In some embodiments, the concentration of a reverse primer having a nucleotide sequence of SEQ ID NO: 135 ranges from 0.05 uM to 1 uM. In some embodiments, the concentration of the reverse primer is 0.5 uM. 0.6 uM, 0.7 uM, 0.8 uM, 0.9 uM, or 1 uM. In some embodiments, the concentration of a probe having a nucleotide sequence of SEQ ID NO: 136 ranges from 0.05 uM to 1 uM. In some embodiments, the concentration of the probe is 0.5 uM. 0.6 uM, 0.7 uM, 0.8 uM, 0.9 uM, or 1 uM. Non-limiting examples of primers and probes that bind specifically to a target nucleic acid sequence from Lactobacillus graseii include the probes in Table X.

In some embodiments, the kits and methods include a pair of primers that bind specifically to a target nucleic acid sequence from Lactobacillus inner. In some embodiment, the pair of primers that bind specifically to a target nucleic acid sequence from Lactobacillus inner include: a pair of primers having a forward primer having a nucleotide sequence of SEQ ID NO: 137, a reverse primer having a nucleotide sequence of SEQ ID NO: 138, and optionally, a probe having a nucleotide sequence of SEQ ID NO: 139.

In some embodiments, the concentration of a forward primer having a nucleotide sequence of SEQ ID NO: 137 ranges from 0.05 uM to 1 uM. In some embodiments, the concentration of the forward primer is 0.5 uM. 0.6 uM, 0.7 uM, 0.8 uM, 0.9 uM, or 1 uM. In some embodiments, the concentration of a reverse primer having a nucleotide sequence of SEQ ID NO: 138 ranges from 0.05 uM to 1 uM. In some embodiments, the concentration of the reverse primer is 0.5 uM. 0.6 uM, 0.7 uM, 0.8 uM, 0.9 uM, or 1 uM. In some embodiments, the concentration of a probe having a nucleotide sequence of SEQ ID NO: 139 ranges from 0.05 uM to 1 uM. In some embodiments, the concentration of the probe is 0.5 uM. 0.6 uM, 0.7 uM, 0.8 uM, 0.9 uM, or 1 uM. Non-limiting examples of primers and probes that bind specifically to a target nucleic acid sequence from Lactobacillus inner include the probes in Table X.

In some embodiments, the kits and methods include a pair of primers that bind specifically to a target nucleic acid sequence from Lactobacillus jenseni. In some embodiment, the pair of primers that bind specifically to a target nucleic acid sequence from Lactobacillus jenseni include: a pair of primers having a forward primer having a nucleotide sequence of SEQ ID NO: 140, a reverse primer having a nucleotide sequence of SEQ ID NO: 141, and optionally, a probe having a nucleotide sequence of SEQ ID NO: 142.

In some embodiments, the concentration of a forward primer having a nucleotide sequence of SEQ ID NO: 140 ranges from 0.05 uM to 1 uM. In some embodiments, the concentration of the forward primer is 0.5 uM. 0.6 uM, 0.7 uM, 0.8 uM, 0.9 uM, or 1 uM. In some embodiments, the concentration of a reverse primer having a nucleotide sequence of SEQ ID NO: 141 ranges from 0.05 uM to 1 uM. In some embodiments, the concentration of the reverse primer is 0.5 uM. 0.6 uM, 0.7 uM, 0.8 uM, 0.9 uM, or 1 uM. In some embodiments, the concentration of a probe having a nucleotide sequence of SEQ ID NO: 142 ranges from 0.05 uM to 1 uM. In some embodiments, the concentration of the probe is 0.5 uM. 0.6 uM, 0.7 uM, 0.8 uM, 0.9 uM, or 1 uM. Non-limiting examples of primers and probes that bind specifically to a target nucleic acid sequence from Lactobacillus jenseni include the probes in Table X.

In some embodiments, the kits and methods include a pair of primers that bind specifically to a target nucleic acid sequence from Gardnerella vaginalis. In some embodiment, the pair of primers that bind specifically to a target nucleic acid sequence from Gardnerella vaginalis include: a pair of primers having a forward primer having a nucleotide sequence of SEQ ID NO: 143, a reverse primer having a nucleotide sequence of SEQ ID NO: 144, and optionally, a probe having a nucleotide sequence of SEQ ID NO: 145.

In some embodiments, the concentration of a forward primer having a nucleotide sequence of SEQ ID NO: 143 ranges from 0.05 uM to 1 uM. In some embodiments, the concentration of the forward primer is 0.5 uM. 0.6 uM, 0.7 uM, 0.8 uM, 0.9 uM, or 1 uM. In some embodiments, the concentration of a reverse primer having a nucleotide sequence of SEQ ID NO: 144 ranges from 0.05 uM to 1 uM. In some embodiments, the concentration of the reverse primer is 0.5 uM. 0.6 uM, 0.7 uM, 0.8 uM, 0.9 uM, or 1 uM. In some embodiments, the concentration of a probe having a nucleotide sequence of SEQ ID NO: 145 ranges from 0.05 uM to 1 uM. In some embodiments, the concentration of the probe is 0.5 uM. 0.6 uM, 0.7 uM, 0.8 uM, 0.9 uM, or 1 uM. Non-limiting examples of primers and probes that bind specifically to a target nucleic acid sequence from Gardnerella vaginalis include the probes in Table X.

In some embodiments, the kits and methods include a pair of primers that bind specifically to a target nucleic acid sequence from Apodium vaginae. In some embodiment, the pair of primers that bind specifically to a target nucleic acid sequence from Apodium vaginae include: a pair of primers having a forward primer having a nucleotide sequence of SEQ ID NO: 146, a reverse primer having a nucleotide sequence of SEQ ID NO: 147, and optionally, a probe having a nucleotide sequence of SEQ ID NO: 148.

In some embodiments, the concentration of a forward primer having a nucleotide sequence of SEQ ID NO: 146 ranges from 0.05 uM to 1 uM. In some embodiments, the concentration of the forward primer is 0.5 uM. 0.6 uM, 0.7 uM, 0.8 uM, 0.9 uM, or 1 uM. In some embodiments, the concentration of a reverse primer having a nucleotide sequence of SEQ ID NO: 147 ranges from 0.05 uM to 1 uM. In some embodiments, the concentration of the reverse primer is 0.5 uM. 0.6 uM, 0.7 uM, 0.8 uM, 0.9 uM, or 1 uM. In some embodiments, the concentration of a probe having a nucleotide sequence of SEQ ID NO: 148 ranges from 0.05 uM to 1 uM. In some embodiments, the concentration of the probe is 0.5 uM. 0.6 uM, 0.7 uM, 0.8 uM, 0.9 uM, or 1 uM. Non-limiting examples of primers and probes that bind specifically to a target nucleic acid sequence from Apodium vaginae include the probes in Table X.

In some embodiments, the kits and methods include a pair of primers that bind specifically to a target nucleic acid sequence from Bactoroides fragilis. In some embodiment, the pair of primers that bind specifically to a target nucleic acid sequence from Bactoroides fragilis include: a pair of primers having a forward primer having a nucleotide sequence of SEQ ID NO: 149, a reverse primer having a nucleotide sequence of SEQ ID NO: 150, and optionally, a probe having a nucleotide sequence of SEQ ID NO: 151.

In some embodiments, the concentration of a forward primer having a nucleotide sequence of SEQ ID NO: 149 ranges from 0.05 uM to 1 uM. In some embodiments, the concentration of the forward primer is 0.5 uM. 0.6 uM, 0.7 uM, 0.8 uM, 0.9 uM, or 1 uM. In some embodiments, the concentration of a reverse primer having a nucleotide sequence of SEQ ID NO: 150 ranges from 0.05 uM to 1 uM. In some embodiments, the concentration of the reverse primer is 0.5 uM. 0.6 uM, 0.7 uM, 0.8 uM, 0.9 uM, or 1 uM. In some embodiments, the concentration of a probe having a nucleotide sequence of SEQ ID NO: 151 ranges from 0.05 uM to 1 uM. In some embodiments, the concentration of the probe is 0.5 uM. 0.6 uM, 0.7 uM, 0.8 uM, 0.9 uM, or 1 uM. Non-limiting examples of primers and probes that bind specifically to a target nucleic acid sequence from Bactoroides fragilis include the probes in Table X.

In some embodiments, the kits and methods include a pair of primers that bind specifically to a target nucleic acid sequence from Mobiliscus curtiis. In some embodiment, the pair of primers that bind specifically to a target nucleic acid sequence from Mobiliscus curtiis include: a pair of primers having a forward primer having a nucleotide sequence of SEQ ID NO: 152, a reverse primer having a nucleotide sequence of SEQ ID NO: 153, and optionally, a probe having a nucleotide sequence of SEQ ID NO: 154.

In some embodiments, the concentration of a forward primer having a nucleotide sequence of SEQ ID NO: 152 ranges from 0.05 uM to 1 uM. In some embodiments, the concentration of the forward primer is 0.5 uM. 0.6 uM, 0.7 uM, 0.8 uM, 0.9 uM, or 1 uM. In some embodiments, the concentration of a reverse primer having a nucleotide sequence of SEQ ID NO: 153 ranges from 0.05 uM to 1 uM. In some embodiments, the concentration of the reverse primer is 0.5 uM. 0.6 uM, 0.7 uM, 0.8 uM, 0.9 uM, or 1 uM. In some embodiments, the concentration of a probe having a nucleotide sequence of SEQ ID NO: 154 ranges from 0.05 uM to 1 uM. In some embodiments, the concentration of the probe is 0.5 uM. 0.6 uM, 0.7 uM, 0.8 uM, 0.9 uM, or 1 uM. Non-limiting examples of primers and probes that bind specifically to a target nucleic acid sequence from Mobiliscus curtiis include the probes in Table X.

TABLE X
Primers and Probes for additional targets

| | Primer/ | SEQ | |
|---|---|---|---|
| Target | Probe | ID NO: | Sequence-5′ - 3′ |
| candida albicans | Forward | 116 | CCTCCACCTTTCACTACTCATC |
| candida albicans | Reverse | 117 | ATGCTGGCACACGGATATT |
| candida albicans | Probe | 118 | AGGTTCATCCACCACCATTCTCCT |
| candida dupliniensis | Forward | 119 | GCGTCGTTTCTCCCTCAAA |
| candida dupliniensis | Reverse | 120 | CATCTCCGCCTTATACCACTATC |
| candida dupliniensis | Probe | 121 | TGGTGTTGAGCAATACGACTTGGGT |
| candida parapsilosis | Forward | 122 | GCGGAAGGATCATTACAGAAT |
| candida parapsilosis | Reverse | 123 | GGTTGAGTTTAATCTCTGGCA |
| candida parapsilosis | Probe | 124 | ACTTTGCTTTGGTAGGCCTTCTATATGGG |
| candida tropicana | Forward | 125 | TTCACTGTTTCTGCTGATCATA |
| candida tropicana | Reverse | 126 | GGCTTTAACTAATTCAATACCAGTC |
| candida tropicana | Probe | 127 | TGGTGGCAACACCTATACTGGTGT |
| candida Africana | Forward | 128 | CTCTACGACTGAAGGTGCTATTC |
| candida Africana | Reverse | 129 | GCACCGAAGGTCAATCTCAT |
| candida Africana | Probe | 130 | TCCACTCATGCAACCATCTGCCAA |
| lactobacillus | Forward | 131 | TGGATCTTAGCAGCTTGCTTTA |
| crispatus | | | |
| lactobacillus | Reverse | 132 | GGGCATGATTATCCCTGTAGTT |
| crispatus | | | |
| lactobacillus | Probe | 133 | AAGAACTGGTGCTGGTGGAGTCTT |
| crispatus | | | |
| lactobacillus graseii | Forward | 134 | CATCTGCAGCGTTTAGATCTTTC |
| lactobacillus graseii | Reverse | 135 | CAAGGGTTCTGGTGAACCTAA |
| lactobacillus graseii | Probe | 136 | TCGGCAATTTCCTTAACTTGGTCCTTAGT |
| lactobacillus inner | Forward | 137 | GCAGCGTTTAGATCTTGCATTT |
| lactobacillus inner | Reverse | 138 | AAAGGATCTGGCGAACCTAATAC |
| lactobacillus inner | Probe | 139 | TGGTTTCAGCGATTTCCTTAACTTGAGC |
| lactobacillus jenseni | Forward | 140 | CTGGCGAACCTAACACTAAGAA |
| lactobacillus jenseni | Reverse | 141 | CGCATTGCTGCTTCAACAT |
| lactobacillus jenseni | Probe | 142 | TCGCTGAAACCAAGATGCAAGACCT |
| Gardnerella | Forward | 143 | GTCGTGTTATGTGAATGGGATTG |
| vaginalis | | | |
| Gardnerella | Reverse | 144 | CCGCGCTACTTGGGAATAA |
| vaginalis | | | |
| Gardnerella | Probe | 145 | AATTCCCAGCCGTAGAGGGTCATG |
| vaginalis | | | |
| Apodium vaginae | Forward | 146 | CCTTACCAGGGCTTGACATTTA |
| Apodium vaginae | Reverse | 147 | CGGGACTTAACCCAACATCTC |
| Apodium vaginae | Probe | 148 | AAGGAGCCTAAACAGGTGGTGCAT |
| Bactoroides fragilis | Forward | 149 | AACTGAGACACGGTCCAAAC |
| Bactoroides fragilis | Reverse | 150 | CCATAGAGCCTTCATCCTTCAC |
| Bactoroides fragilis | Probe | 151 | ATATTGGTCAATGGGCGCTAGCCT |
| Mobiliscus curtiis | Forward | 152 | GTACGGTCGCAAGGCTAAA |
| Mobiliscus curtiis | Reverse | 153 | GGAACCATCGCAGTGTATGT |
| Mobiliscus curtiis | Probe | 154 | ACGCGAAGAACCTTACCAAGGCTT |

### Primers and Probes for Controls Nucleic Acid Sequences

In some embodiments, the kits and methods provided herein include a pair of primers that bind specifically to a target nucleic acid sequence from a control population of nucleic acid sequences.

In some embodiments, the pair of primers that that bind specifically to a target nucleic acid sequence from a control population of nucleic acid sequences include a forward primer having SEQ ID NO: 155, a reverse primer having SEQ ID NO: 156, and optionally, a probe having SEQ ID NO: 157. In some embodiments, the concentration of a forward primer having a nucleotide sequence of SEQ ID NO: 155 ranges from 0.05 uM to 1 uM. In some embodiments, the concentration of the forward primer is 0.5 uM. 0.6 uM, 0.7 uM, 0.8 uM, 0.9 uM, or 1 uM. In some embodiments, the concentration of a reverse primer having a nucleotide sequence of SEQ ID NO: 156 ranges from 0.05 uM to 1 uM. In some embodiments, the concentration of the reverse primer is 0.5 uM. 0.6 uM, 0.7 uM, 0.8 uM, 0.9 uM, or 1 uM. In some embodiments, the concentration of a probe having a nucleotide sequence of SEQ ID NO: 157 ranges from 0.05 uM to 1 uM. In some embodiments, the concentration of the probe is 0.5 uM. 0.6 uM, 0.7 uM, 0.8 uM, 0.9 uM, or 1 uM. Non-limiting examples of probes that bind specifically to a target nucleic acid sequence of a control nucleic acid sequence (e.g., inactivated cells) include the probes in Table 7.

TABLE 7
Primers and Probes for Control
Nucleic Acid Sequences

| | | SEQ | |
|---|---|---|---|
| | Primer/ | ID | |
| Target | Probe | NO: | Sequence-5′ - 3′ |
| RNaseP | Forward | 155 | AGATTTGGACCTGCGAGCg |
| RNaseP | Reverse | 156 | GAGCGGCTGTCTCCACAAGt |
| RNaseP | Probe | 157 | TTCTGACCTGAAGGCTCTGCGCG |

### Detectably-Labeled Probes

In some embodiments, the kits and methods described herein include detectable labels attached (e.g., bound) to a probe. In some embodiments, the first pair of primers, the second pair of primers, the third pair of primers, the fourth pair of primers, the optional probes, or a combination thereof include detectably labeled primers and/or probes, wherein the probe(s) bind (hybridize) specifically to the respective target nucleic acid sequence. In some embodiments, the detectably labeled probes comprise a dual-labeled probe.

Non-limiting examples of detectably-labeled probes and quenchers include: 5′ reporter dye/emission (nm): FAM 520 and quencher: ZEN/Iowa Black FQ*; reporter dye/emission (nm): FAM 520 and quencher: TAMRA/Black Hole Quencher 1; reporter dye/emission (nm): TET 529 and quencher: ZEN/Iowa Black FQ* (Black Hole Quencher 1); reporter dye/emission (nm): YAK 549 and quencher: ZEN/Iowa Black FQ*; reporter dye/emission (nm): SUN 554 and quencher: ZEN/Iowa Black FQ*—Iowa Black FQ; or reporter dye/emission (nm): HEX 555 and quencher: ZEN/Iowa Black FQ*; ZEN/Iowa Black FQ */Black Hole Quencher 1. As used above, * indicates ZEN/Iowa Black™ FQ is a double-quenched probe, which provides superior performance compared to traditional single-quenched probes.

### Sexual Health Conditions

In some embodiments, the methods include detection of a sexual health condition, where the sexual health condition is associated with at least one of: Chlamydia trachomatis, Neisseria gonorrhoeae, and Trichomonas vaginalis, Treponema pallidum, Gardnerella vaginitis, Candida Albicans, Mycoplasma genitalium, Human papillomavirus (HPV), Herpes simplex viruses (HSV), Hepatitis B, and human immunodeficiency virus (HIV). In some embodiments, the methods include detection of a sexual health condition, where the sexual health condition is associated with at least one of: Chlamydia trachomatis, Neisseria gonorrhoeae, and Trichomonas vaginalis. In some embodiments, the methods include detection of a sexual health condition, where the sexual health condition is associated with at least two of: Chlamydia trachomatis, Neisseria gonorrhoeae, and Trichomonas vaginalis.

Chlamydia trachomatis

In some embodiments, the sexual health condition is an infection associated with a strain of Chlamydia trachomatis. In one embodiment, the system is configured to detect presence of Chlamydia trachomatis from a sample acquired from the subject, such that the target material includes one or more specific regions of biological material (e.g., nucleic acid content) of Chlamydia trachomatis, and primer and probes are configured to bind to specific regions of Chlamydia trachomatis.

Non-limiting examples of Chlamydia trachomatis strains are listed in Table 8.

TABLE 8
Example Serovars/Strains Chlamydia trachomatis

| Organism | Strain | Product info. |
|---|---|---|
| [symbol] | LGV-2-434 | Microbiologics |
| | | HE0035N |
| | Serovar C | ATCC VR-1477 |
| | Serovar D | ATCC VR-885 |
| | Serovar E | ATCC VR-348B |
| | Serovar F | ATCC VR-346 |
| | Serovar G | ATCC VR-878 ™ |
| | Serovar H | ATCC VR-879 ™ |
| | Serovar I | ATCC VR-880 ™ |
| | Serovar J | ATCC VR-886 ™ |
| | Serovar K | ATCCVR-887 ™ |

[symbol]

indicates data missing or illegible when filed

Neisseria gonorrhoeae

In some embodiments, the sexual health condition is an infection associated with a strain of Neisseria gonorrhoeae. In one embodiment, the system is configured to detect presence of Neisseria gonorrhoeae from a sample acquired from the subject, such that the target material includes one or more specific regions of biological material (e.g., nucleic acid content) of Neisseria gonorrhoeae, and the primers and probes are configured to bind to specific regions of Neisseria gonorrhoeae.

In some embodiments, the target nucleic acid sequence of Neisseria gonorrhoeae encodes for individual proteins (and homologs) or a cocktails of proteins (and homologs) including those as described in PCT Publication WO2022031560, which is herein incorporated by reference in its entirety. For example, individual proteins (and homologs) or a cocktails of proteins (and homologs) as described in WO2022031560 include one or more of: Uniprot ID numbers: P95359 (SEQ ID NO 23 of WO2022031560), A0A1D3HF49 (SEQ ID NO 24 of WO2022031560), P05430 (SEQ ID NO 25 of WO2022031560), Q02219 (SEQ ID NO 26 of WO2022031560), Q51006 (SEQ ID NO 27 of WO2022031560), Q5F942 (SEQ ID NO 28 of WO2022031560), B4RQH9 (SEQ ID NO 29 of WO2022031560), Q5F6W5 (SEQ ID NO 30 of WO2022031560), P29842 (SEQ ID NO 31 of WO2022031560), Q5F542 (SEQ ID NO 32 of WO2022031560), B4RLT9 (SEQ ID NO 33 of WO2022031560), D6H5Z3 (SEQ ID NO 34 of WO2022031560), and Q5F651 (SEQ ID NO 35 of WO2022031560); and GenBank/NCBI Accession Numbers: YP_208979.1 (SEQ ID NO 75 of WO2022031560), KX124787.1 (SEQ ID NO 76 of WO2022031560), SCW17313.1 (SEQ ID NO 77 of WO2022031560), YP_209073.1 (SEQ ID NO 78 of WO2022031560), and YP_209148.1 (SEQ ID NO 79 of WO2022031560). In some embodiments, the target material of Neisseria gonorrhoeae includes lipoglycans or lipopolysaccharides of the Neisseria gonorrhoeae species.

Non-limiting examples of Neisseria gonorrhoeae strains are listed in Table 9.

TABLE 9
Example Serovars/Strains Neisseria gonorrhoeae

| Organism | Strain | Product info. |
|---|---|---|
| Neisseria | AR-0166 | CDC |
| gonorrhoeae | AR-0168 | CDC |
| | AR-0205 | CDC |
| | AR-0206 | CDC |
| | AR-0207 | CDC |
| | AR-0208 | CDC |
| | AR-0209 | CDC |
| | AR-0210 | CDC |
| | AR-0211 | CDC |
| | AR-0212 | CDC |
| | AR-0213 | CDC |
| | AR-0214 | CDC |
| | CDC Ng-98 | ATCC 43070 |
| | CDC Ng-116 | ATCC 43069 |
| | | ATCC 19424 |
| | LQ55 < 6 | R [symbol] |
| | | MJ5596S |

[symbol]

indicates data missing or illegible when filed

Trichomonas vaginalis

In some embodiments, the sexual health condition is an infection associated with a strain of Trichomonas vaginalis. In one embodiment, the system is configured to detect presence of Trichomonas vaginalis from a sample acquired from the subject, such that the target material includes one or more specific regions of biological material (e.g., nucleic acid content) of Trichomonas vaginalis, and the primers and probes are configured to bind to specific regions of Trichomonas vaginalis.

In some embodiments, the target nucleic acid sequence of Trichomonas vaginalis encodes individual proteins (and homologs) or a cocktails of proteins (and homologs) including those as described in PCT Publication WO2022031560, which is herein incorporated by reference in its entirety. For example, individual proteins (and homologs) or a cocktails of proteins (and homologs) as described in WO2022031560 includes one or more of: NCBI/GenBank Accession numbers: EAX87747.1 (SEQ ID NO 41 of WO2022031560), EAY21310.1 (SEQ ID NO 42 of WO2022031560), EAX96596.1 (SEQ ID NO 43 of WO2022031560), EAY19137.1 (SEQ ID NO 44 of WO2022031560), EAY01676.1 (SEQ ID NO 45 of WO2022031560), EAX86868.1 (SEQ ID NO 46 of WO2022031560), EAX98121.1 (SEQ ID NO 47 of WO2022031560), EAY18961.1 (SEQ ID NO 48 of WO2022031560), AAA91133.1 (SEQ ID NO 49 of WO2022031560), AAC48339.1 (SEQ ID NO 50 of WO2022031560), and AAC72899.1 (SEQ ID NO 51 of WO2022031560). In some embodiments, the target material of Trichomonas vaginalis includes lipoglycans or lipopolysaccharides of the Trichomonas vaginalis species

Non-limiting examples of Trichomonas vaginalis strains are listed in Table 10.

TABLE 10
Example Serovars/Strains Chlamydia trachomatis, Neisseria
gonorrhoeae, and Trichomonas vaginalis

| Organism | Strain | Product info. |
|---|---|---|
| Trichomonas | 165307-1 | ATCC 30187 |
| vaginalis | JH 32A #2 | ATCC 30237 |
| | 11769 | ATCC 30092 |
| | NYH 286 | ATCC 50148 |
| | RU 357 | ATCC 50139 |
| | | ATCC 30238 |
| | MT87 | ATCC PRA-96 |
| | | ATCC 50747 |
| | HsD:NIH | ATCC 50183 |
| | TVC | ATCC 30245 |
| | NYH 286 | ATCC 50148 |
| | LQ557= | R [symbol] MJ5597S |

[symbol]

indicates data missing or illegible when filed

### Kit Identification

In various embodiments, each kit has an ID or code associated with it. This ID or code would encapsulate multiple variables which could identify the kit and components within the kit, such as, but not limited to, the corresponding reagents with information including: Lot ID, Batch ID, Test ID, Expiration Date, Seller, Re-seller information, etc. Capture of the ID could be either based on QR code or user input. In some embodiments, the code or ID can provide information about the user. The ID can help provide additional services to the end user. Some of the services may include, but are not limited to, connecting to telehealth, various helplines, clinics, prescriptions, and the like. The output of the system, kit and/or methods include a readout on the presence or absence of a sexual health condition in a sample. This output can be uploaded to a medical site, an online portal, mobile or computer application accessible by one or more individuals who either provided the sample or who can act on behalf of the individual who provided the sample. In some embodiments, the online portal can provide virtual communication and telemedicine to the user from a healthcare provider to facilitate further patient care following output of the readout on the presence or absence of a sexual health condition in a sample. Such patient care can include, but is not limited to, a prescription to treat a diagnosis following output of the results, follow-up testing, and the like. In some embodiments, the medical site mobile or computer application enables users to upload additional information, for example, the results from a test kit as described in PCT Publication No. WO 2022/031560, which is herein incorporated by reference. In some embodiments, the kit and/or methods described herein are used in conjunction with or to supplement the kit as described in WO 2022/031560. In such cases, the kit and/or methods described herein can be used to confirm a result first reported from a test kit from WO 2022/031560.

In some embodiments, the medical team (e.g., clinician) can read the results, send comments to the user, send an interpretation of the test results, among other interactions. In one embodiment, the sample kit includes a telehealth system that allows the physician to interact with the user including scheduling a video conference or phone call to discuss the test results. In one embodiment, the medical team can prescribe medication or therapy to treat the condition if the user has a positive test. The physician can send the prescription to the pharmacy based on the test results received through the mobile application. The user can coordinate the delivery or pickup of the prescription through the mobile application, and coordinate refills of the prescription with the medical team and pharmacy. In one embodiment, the system is an end-to-end computer telehealth system where a medical team can prescribe to the user a test in which the user receives the sampling kit in the mail or from the store.

### Methods of Detecting at Least One Sexual Health Condition

Provided herein are methods for detecting status of at least one sexual health condition (e.g., any of the sexual health conditions described herein). In some embodiments, the method is multiplex, meaning the method detects two or more sexual health conditions (e.g., any two or more the sexual health conditions described herein). In some embodiments, the method is monoplex, meaning the method detects one sexual health condition (e.g., any one of the sexual health conditions described herein. In some embodiments, a monoplex method is used in conjunction with a multiplex method. For example, a multiplex method is used first to detect the presence or absence of a sexual health condition in a sample. The same sample is subject to a monoplex method. In some cases, the monoplex method is used to confirm the results of a multiplex method. In other cases, the monoplex method is used to gather additional information on the one or more sexual health conditions identified by the multiplex reaction.

Provided herein is a method for detecting status of at least one sexual health condition, the method comprising: (a) receiving a sample acquired directly from a subject; (b) extracting or purifying a plurality of target nucleic acid sequences from the sample using an extraction buffer (e.g., any of the extraction buffers described herein); (c) amplifying a target nucleic acid sequence of plurality of target nucleic acid sequences by PCR amplification using primers and an amplification reaction mixture, wherein the primers comprise at least one of: (i) a first pair of primers (e.g., any of the primers described in Table 1) that bind specifically to a target nucleic acid sequence from Chlamydia trachomatis, (ii) a second pair of primers (e.g., any of the primers described in Table 2) that bind specifically to a target nucleic acid sequence from Neisseria gonorrhoeae, (iii) a third pair of primers (e.g., any of the primers described in Table 3) that bind specifically to a target nucleic acid sequence from Trichomonas vaginalis, or (iv) a combination thereof; and (d) detecting the presence or absence of the PCR amplification product, thereby determining status of at least one sexual health condition. In some embodiments, the method also includes a pair of primers (e.g., any of the primers described in Tables 7) that bind specifically to a fourth target nucleic acid sequence from a control population of nucleic acid sequences.

Provided herein is a method for detecting status of at least one sexual health condition, the method comprising: (a) receiving a sample acquired directly from a subject; (b) extracting or purifying a plurality of target nucleic acid sequences from the sample using an extraction buffer (e.g., any of the extraction buffers described herein); (c) amplifying a target nucleic acid sequence of plurality of target nucleic acid sequences by PCR amplification using primers and an amplification reaction mixture, wherein the primers comprise: (i) a pair of primers (e.g., any of the primers described in Table 3) that bind specifically to a target nucleic acid sequence from Chlamydia trachomatis, and a pair of primers (e.g., any of the primers described in Table 7) that bind specifically to a nucleic acid sequence from a control population of nucleic acid sequences, and (d) detecting the presence or absence of the PCR amplification product, thereby determining status of at least one sexual health condition.

Provided herein is a method for detecting status of at least one sexual health condition, the method comprising: (a) receiving a sample acquired directly from a subject; (b) extracting or purifying a plurality of target nucleic acid sequences from the sample using an extraction buffer; (c) amplifying a target nucleic acid sequence of plurality of target nucleic acid sequences by PCR amplification using primers and an amplification reaction mixture, wherein the primers comprise: (i) a pair of primers (e.g., any of the primers described in Table 3) that bind specifically to a target nucleic acid sequence from Neisseria gonorrhoeae, and a pair of primers (e.g., any of the primers described in Table 7) that bind specifically to a nucleic acid sequence from a control population of nucleic acid sequences, and (d) detecting the presence or absence of the PCR amplification product, thereby determining status of at least one sexual health condition.

Provided herein is a method for detecting status of at least one sexual health condition, the method comprising: (a) receiving a sample acquired directly from a subject; (b) extracting or purifying a plurality of target nucleic acid sequences from the sample using an extraction buffer (e.g., any of the extraction buffers described herein); (c) amplifying a target nucleic acid sequence of plurality of target nucleic acid sequences by PCR amplification using primers and an amplification reaction mixture, wherein the primers comprise: (i) a pair of primers (e.g., any of the primers described in Table 6) that bind specifically to a target nucleic acid sequence from Trichomonas vaginalis, and a pair of primers (e.g., any of the primers described in Table 7) that bind specifically to a nucleic acid sequence from a control population of nucleic acid sequences, and (d) detecting the presence or absence of the PCR amplification product, thereby determining status of at least one sexual health condition.

### Sample Collection and Receiving the Sample Acquired From a Subject

In some embodiments, the sample is acquired directly from the subject using a sample collection tool (e.g., any of the sample collection tools described herein or known in the art). In some embodiments, the sample is collected directly from the subject at-home or at a clinic. In some embodiments, the sample is collected at-home using the at-home kit described herein. In some embodiments, the sample is collected at a clinical using the at-home kit described herein.

In some embodiments, the sample is a body fluid and/or tissue sample (e.g., a mucosa tissue sample, a urine sample, a blood sample, etc.). In some embodiments, the sample includes vaginal discharge or penile discharge for sexual health analysis. In some embodiments, the sample is a urine sample, or a sample taken from swabs from throat, rectal (e.g. anorectal), vaginal, endocervical and urethral sites. In some embodiments, the sample includes a sweat sample. In some embodiments, the samples can be a combination of one or more samples selected from: a tissue sample, a urine sample, a blood sample, an oral fluid sample, a vaginal sample, a penile sample, and a rectal sample. In some embodiments, the samples can be a combination of two or more samples selected from: a urine sample, a blood sample, an oral sample, a vaginal sample, a penile sample, a rectal sample, a sample from the bronchoalveolar lavage, saliva, buccal, nasal, tears, serum, plasma, skin, stool, sweat, synovial fluid, wound fluid, dental scapings, and the like. In some embodiments, the samples can be a combination of a urine sample, an oral sample, and a rectal sample. In some embodiments, more than one sample collecting tool is used to collect multiple samples from different regions of the body. For example, if 2 samples are collected from the user in 2 different areas of the body, then two sample collecting cools or sample collecting devices are used to collect the sample, depending on the nature of the sample. As a non-limiting example, a first sample collection device such as a collapsible cup can be used to collect a urine sample, and a second sample collection device or cool comprising a sample swab can be used to collect an oral, a vaginal, or a rectal sample. In certain embodiments, the multiple samples collected are placed in a single storage or collection container. In certain embodiments, the multiple samples collected are placed in a separate storage or collection containers. In certain embodiments, the multiple samples are placed in a separate storage or collection container and then transferred to a single container when processing the sample in a laboratory.

In a non-limiting example, the sample is a urine specimen. FIGS. 1A-1B provide a non-limiting example of a method for collecting a sample directly from the subject at-home using a sample collection tool where the sample is a urine specimen. In step 1, urine is collected in urine collection cup. In step 2, a transfer pipette is used to collect urine from the urine collection cup. In step 3, the transfer pipette is used to transfer the urine to the urine collection tube. In some embodiments, the urine collection tube is shipped to a second location for further processing. For example, the urine collection tube is placed in a biohazard bag (SHIP THE SAMPLE, step 1), the biohazard bag is placed into a shipping envelope (SHIP THE SAMPLE, step 2), and the envelope is shipped.

In some embodiments, the sample is acquired from the subject by a clinician. In some embodiments, the sample is acquired from the subject by the patient or consumer at-home.

In some embodiments, the sample is received in a sample collection container. In some embodiments, DNA extraction is performed in the sample collection container. In some embodiments, quantitative PCR is performed on the sample without having to extract the DNA using an extraction buffer.

In some embodiments, a sample collected by a sample collecting tool includes vaginal fluid, vaginal tissue, vaginal washing, vaginal swab, vaginal discharge, cervical swab, cervical tissue urethral swab, urethral discharge, rectal swab, rectal material, rectal washing, oral swab (e.g., to collect saliva), urine, blood, serum, plasma, saliva, tears, skin swab, semen, seminal fluid, sputum, bronchial fluid, bronchial washing, peritoneal fluid, peritoneal washing, pleural fluid, pleural washing, cerebrospinal fluid, nasal swabs and/or fluids, throat swabs and/or fluids eye fluid and/or tissue, fluid and/or tissue from lung, liver, heart, brain, kidney, spleen or muscle and any combination thereof. In some embodiments, the sample is a blood sample, and the sample collecting tool is, e.g., a lancet for piercing a finger and a container or slide for collecting the blood sample). In some embodiments, the sample is a urine sample, and the sample collecting tube is, e.g., a urine collection cup, urine swab, etc. In some embodiments, the sample is a vaginal discharge or a penile discharge, and the sample collecting tool is a swab, cotton pad, sample container, etc. In some embodiments, the sample is obtained from contacting an ulcer in genital area, and the sample collecting tool is a swab, cotton pad, sample container, etc.

In some embodiments, when a swab is used, the swab does not contain a buffer (e.g., is dry before applying on the user to collect the sample).

In some embodiments, the sample can be preabsorbed, e.g., to reduce or minimize cross-reactivity and/or background. As nonlimiting examples, in some embodiments, the biological sample can be preabsorbed with a lysate of bacteria expressing glutathione-S-transferase (GST) and/or a lysate of normal (e.g., non-pathogen infected mammalian cells). In some embodiments, absorption of the sample can be with a lysate of pathogen-infected mammalian cells, to remove and/or block chlamydial antigen-specific antibodies from human samples, which can help confirm the specificity of human antibody binding to a test analyte.

In some embodiments, the sample collecting tool comprises a fluid collecting container. In some embodiments, the fluid collecting container comprises a tube. In some embodiments, the tube is serum tube or a plasma tube.

In some embodiments, the sample is or is recommended to be collected at a specific time or in a specific period of time. In some embodiments, the sample is or is recommended to be collected in the morning. In some embodiments, the sample is or is recommended to be collected within 1, 2, 3, 4, 5, 6, or more hours before urinating. In some embodiments, the sample is or is recommended to be collected at noon. In some embodiments, the sample is or is recommended to be collected in the evening. In some embodiments, the sample is or is recommended to be collected before the shower. In some embodiments, the sample is or is recommended to be collected before the individual having sex. In some embodiments, the sample is or is recommended to be collected after the individual having sex. In some embodiments, the sample is or is recommended to be collected within 1, 2, 3, 4, 5, 6 or more hours before or after the individual having sex. In some embodiments, the sample is or is recommended to be collected at least 4, 5, 6, 7, 8, 9, 10, 12 days after the individual ovulates.

In some embodiments, the biological sample is stable at room temperature for at least 1, 2, 4, 8, 12, 16, 20, 24 hours after obtained. In some embodiments, the sample is or is recommended to be tested within 1, 2, 3, 4, 5, 6 hours after it is obtained. In some embodiments, the sample is or is recommended to be tested shortly after it is obtained (for example, within an hour).

In some embodiments, samples that include interfering substances (e.g., any of interfering substances described herein or known in the art) can still be used with the kits provided herein and subjected to the methods provided herein.

In some embodiments, the methods include detection of a sexual health condition, where the sexual health condition is an infection associated with Chlamydia trachomatis. In some embodiments, the sexual health condition is an infection associated with a strain of Chlamydia trachomatis. Non-limiting examples of Chlamydia trachomatis strains are listed in Table 6.

In some embodiments, the methods include detection of a sexual health condition, where the sexual health condition is an infection associated with Neisseria gonorrhoeae. In some embodiments, the sexual health condition is an infection associated with a strain of Neisseria gonorrhoeae. Non-limiting examples of Neisseria gonorrhoeae strains are listed in Table 7.

In some embodiments, the methods include detection of a sexual health condition, where the sexual health condition is an infection associated with Trichomonas vaginalis. In some embodiments, the sexual health condition is an infection associated with a strain of Trichomonas vaginalis. Non-limiting examples Trichomonas vaginalis strains are listed in Table 8.

In some embodiments, the method includes a buffer (e.g., such as an extraction buffer and/or a storage buffer). In some embodiments, the buffer includes phosphate buffered saline, Tris-buffered saline, HEPES buffered saline, a reducing agent, sodium hydroxide, Triton X-100, and octyl glucoside. In some embodiments, the buffer is any collection, storage, and/or transport solution configured to preserve a gynecologic sample. In some embodiments, the sample is a PreservCyt™ solution. In some embodiments, the buffer is a CytoLyt solution. In some embodiments, the buffer is a methanol-based, buffered transport solution used in sample preparation prior to processing for analysis.

### Extraction or Purification of Target Nucleic Acids

In some embodiments, the target nucleic acids are extracted, isolated, or purified from the sample. Extraction or purification of the target nucleic acids can be performed using a variety of conventional techniques. For example, in some embodiments, the target nucleic acids are extracted using an extraction buffer and/or a lysis buffer. In some embodiments, the target nucleic acids are extracted using a spin-column based method. In the case where the target nucleic acids are purified with a spin-column containing a filter on which the nucleic acids comprising the target nucleic acid is immobilized, an elution solution may be added to the spin column which is then centrifuged to cause the elution solution to elute the nucleic acids comprising DNA from the solid support for collection. In some embodiments, the step of enriching the sample comprises: filtering the biological sample through a filter, wherein fluid passed through the filter is stored in a waste chamber and the remainder of the biological sample remains in the sample chamber; or (b) magnetically labeling the targets of interest within the sample and subjecting the labelled sample to a magnetic field. In some embodiments, purifying the target nucleic acids comprises lysing the sample by (a) applying heat to the filter and the biological sample to produce a lysate; or (b) subjecting the biological sample to an electric field to cause electroporation. Furthermore, the steps of adding a sample to a filter through obtaining a lysate can comprise using a spin column filtration configuration or a syringe based cartridge.

In some embodiments, the target nucleic acids are extracted using a high pure PCR template preparation kit. Any extraction and/or purification technique can be used to isolate the target nucleic acids of interest from the sample. In some embodiments, target nucleic acids are isolated from the sample using a DNA isolation kit.

In some embodiments, the target nucleic acids are purified from the sample using gel filtration chromatography.

### Amplification

In some embodiments, the amplification reaction mixture comprises a polymerase comprising 5′ nuclease activity (e.g., any polymerase that comprises a 5′ endonuclease activity described herein). Non-limiting examples of polymerase comprising a 5′ nuclease activity include: Taq DNA polymerase, DNA polymerase I, ϕ29 DNA polymerase, Bst DNA Polymerase, and the like

In some embodiments, the method includes a pre-amplification, where target nucleic acid sequences from one or more of the sexual health conditions is amplified in a pre-amplification step before a second amplification step, where the second amplification step is associated with the detecting step.

In some embodiments, the first pair of primers, the second pair of primers, the third pair of primers, the fourth pair of primers, or a combination thereof each include a probe, where the probe binds specifically to the respective target nucleic acid sequence.

In some embodiments, the first pair of primers, the second pair of primers, the third pair of primers, the fourth pair of primers, probes, or a combination thereof include detectably labeled primers and/or probes, wherein the probe(s) bind specifically to the respective target nucleic acid sequence.

In some embodiments, where the method is a multiplex reaction and the amplifying step occurs in a single well. In certain embodiments, the method is a multiplex reaction and the amplifying step occurs in a single reaction. In some embodiments, the target nucleic acids from the sample, and the primers for detecting a first target (e.g., Chlamydia trachomatis), and at least the primers for detecting a second target (e.g., Neisseria gonorrhoeae), are detected simultaneously in a single panel. In certain embodiments, the method is a multiplex reaction and the amplifying step occurs in a single reaction. In some embodiments, the target nucleic acids from the sample, and the primers for detecting a first target (e.g., Chlamydia trachomatis), and the primers for detecting a second target (e.g., Neisseria gonorrhoeae), and the primers for detecting at least a third target (e.g., Trichomonas vaginalis) are detected simultaneously in a single panel. This method can significantly reduce the time it takes for determining whether the user has one or more sexual health conditions, and thus displaying the results to the user in a more time effective and efficient manner. In some embodiments, the method of the present disclosure performs the amplifying step using multiplexed PCR. In some embodiments, the method of the present disclosure can detecting one or more targets, two or more targets, three or more targets, four or more targets, five or more targets, six or more targets, seven or more targets, eight or more targets, nine or more targets, or ten or more targets in a single reaction e.g., during multiplexed PCR.

In some embodiments, the concentration of the extracted or purified DNA template suspected to contain the target nucleic acid in the sample used in the amplification reaction ranges from 1-μl-10 μl. In some embodiments, the concentration of the extracted or purified DNA template suspected to contain the target nucleic acid in the sample used in the amplification reaction is at least 1 μl, at least 2 μl, at least 3 μl, at least 4 μl, at least 5 μl, at least 6 μl, at least 7 μl, at least 8 μl, at least 9 μl, at least 10 μl, at least 11 μl, at least 12 μl, at least 13 μl, at least 14 μl, or at least 15 μl.

In some embodiments, the amplification reaction mixture (e.g., PCR mastermix) comprises a concentration ranging from 5-μl-10 μl. In some embodiments, the amplification reaction mixture comprises a concentration of at least at least 5 μl, at least 6 μl, at least 7 μl, at least 8 μl, at least 9 μl, at least 10 μl, at least 11 μl, at least 12 μl, at least 13 μl, at least 14 μl, or at least 15 μl.

In some embodiments, amplification occurs in a single well containing the amplification reaction mixture, the extracted or purified DNA template from the sample, a first pair of primers that bind specifically to a target nucleic acid sequence from Chlamydia trachomatis where present in the sample, a second pair of primers that bind specifically to a target nucleic acid sequence from Neisseria gonorrhoeae, where present in the sample, a third pair of primers that bind specifically to a target nucleic acid sequence from Trichomonas vaginalis where present in the sample. In some embodiments, the first pair of primers, the second pair of primers, and the third pair of primers each include a probe, wherein the probe binds specifically to the respective target nucleic acid sequence. In some embodiments, the single well comprises a total volume ranging from 10 μl to 20 μl of an amplification reaction mixture, extracted or purified DNA template from the sample, a first pair of primers and associated probes, a second pair of primers and associated probes, and a third pair of primers and associated probes (see e.g., Table 11). In some embodiments, the single well comprises a total volume ranging from 10 μl to 50 μl of an amplification reaction mixture, extracted or purified DNA template from the sample, a first pair of primers and associated probes, a second pair of primers and associated probes, and a third pair of primers and associated probes (see e.g., Table 11). In some embodiments, the total volume is at least 10 μl, at least 11 μl, at least 12 μl, at least 13 μl, at least 14 μl, at least 15 μl, at least 16 μl, at least 17 μl, at least 18 μl, at least 19 μl, at least 20 μl, at least 21 μl, at least 22 μl, at least 23 μl, at least 24 μl, at least 25 μl, at least 26 μl, at least 27 μl, at least 28 μl, at least 29 μl, or at least 30 μl.

In some embodiments, the single well comprises a total volume ranging from 10 μl to 20 μl of an amplification reaction mixture, extracted or purified DNA template from the sample, a first pair of primers, a second pair of primers, and a third pair of primers (see e.g., Table 11). In some embodiments, the single well comprises a total volume ranging from 10 μl to 50 μl of an amplification reaction mixture, extracted or purified DNA template from the sample, a first pair of primers, a second pair of primers, and a third pair of primers. In some embodiments, the total volume is at least 10 μl, at least 11 μl, at least 12 μl, at least 13 μl, at least 14 μl, at least 15 μl, at least 16 μl, at least 17 μl, at least 18 μl, at least 19 μl, at least 20 μl, at least 21 μl, at least 22 μl, at least 23 μl, at least 24 μl, at least 25 μl, at least 26 μl, at least 27 μl, at least 28 μl, at least 29 μl, or at least 30 μl.

In some embodiments, the method includes detecting at least two sexual health conditions based on the presence or absence of the PCR amplification product.

In some embodiments, the method includes detecting three sexual health conditions based on the presence or absence of the PCR amplification product.

In some embodiments, detecting the presence of at least one sexual health conditions is based on the presence of at least one: Chlamydia trachomatis strain (e.g., any of the Chlamydia trachomatis strains listed in Table 6), Neisseria gonorrhoeae strain (e.g., any of the Neisseria gonorrhoeae strains listed in Table 7), or Trichomonas vaginalis strain (e.g., any of the Trichomonas vaginalis strains listed in Table 8), in the PCR amplification product.

In some embodiments, detecting the presence of at least two sexual health conditions is based on the presence of at least two strains selected from: Chlamydia trachomatis (e.g., any of the Chlamydia trachomatis strains listed in Table 6), Neisseria gonorrhoeae (e.g., any of the Neisseria gonorrhoeae strains listed in Table 7), and Trichomonas vaginalis (e.g., any of the Trichomonas vaginalis strains listed in Table 8), in the PCR amplification product.

In some embodiments, detecting the presence of at least three sexual health conditions is based on the presence of at least three strains selected from: Chlamydia trachomatis (e.g., any of the Chlamydia trachomatis strains listed in Table 6), Neisseria gonorrhoeae (e.g., any of the Neisseria gonorrhoeae strains listed in Table 7), and Trichomonas vaginalis (e.g., any of the Trichomonas vaginalis strains listed in Table 8), in the PCR amplification product.

In some embodiments, the method further comprises delivering a test result from step (d) to the subject via an online portal.

### Method—Detection

In some embodiments, the methods of the present disclosure includes detecting the presence or absence of the PCR amplification product. In such cases, detecting the presence or absence of the PCR amplification product enables determination of the status of at least one sexual health condition, for example, using a PCR thermocycler.

In one or more embodiments, the data output by the PCR thermocycler is provided to a computing device for analysis and detection of the PCR amplification product. The data output by the PCR thermocycler may include quantification of PCR amplification product over time. The quantification may be represented as an amplification curve measured over time. The amplification curve is a graphical representation of the fluorescence intensity (plotted on the y-axis) against the number of PCR cycles (plotted on the x-axis). Each amplification curve can be distinguished as associated with a particular pair of primers targeting a nucleic acid sequence based on the fluorescence signal. For example, a first pair of primers targeting a first nucleic acid sequence includes a probe for binding of fluorophores that fluoresce at a first wavelength of light, whereas a second pair of primers targeting a second nucleic acid sequence includes a probe for binding of fluorophores that fluoresce at a second wavelength of light.

The computing device can analyze the output data to determine whether there is presence of the target nucleic acid sequences. In one or more embodiments, the computing device may compare the amplification curves associated with the target nucleic acid sequences against one or more control amplification curves. The control amplification curve may pertain to expected presence of DNA molecules from the sample. In other embodiments, the control amplification curves pertain to a step ladder of known DNA concentrations. In some embodiments, the computing device may determine whether the amplification curves cross a minimum threshold fluorescence intensity (e.g., relative fluorescence unit (RFU)). In other embodiments, the computing device may determine a relative concentration of the target nucleic acid sequences by comparing the amplification curves of the target nucleic acid sequences to the control amplification curve.

### Method—Sensitivity and Specificity

In some embodiments, the method achieves a limit of detection for determining the presence of a target nucleic acid sequence from Chlamydia trachomatis of at least about 0.5 copies per reaction (cp/rxn), 0.6 cp/rxn, 0.7 cp/rxn, 0.8 cp/rxn, 0.9 cp/rxn, 1.0 cp/rxn, 1.1 cp/rxn, 1.2 cp/rxn, 1.3 cp/rxn, 1.4 cp/rxn, 1.5 cp/rxn, 1.6 cp/rxn, 1.7 cp/rxn, 1.8 cp/rxn, 1.9 cp/rxn, 2.0 cp/rxn, 3 cp/rxn, 4 cp/rxn, 5 cp/rxn, 6 cp/rxn, 7 cp/rxn, 8, cp/rxn, 9 cp/rxn, 10 cp/rxn, 11 cp/rxn, 12 cp/rxn, 13 cp/rxn, 14 cp/rxn, 15 cp/rxn, 16 cp/rxn, 17 cp/rxn, 18 cp/rxn, 19 cp/rxn, 20 cp/rxn, 25 cp/rxn, 30 cp/rxn, 35 cp/rxn, 40 cp/rxn, 45 cp/rxn, 50 cp/rxn, 55 cp/rxn, 60 cp/rxn, 65 cp/rxn, 70 cp/rxn 75 cp/rxn, 80 cp/rxn, 85 cp/rxn, 90 cp/rxn, 95 cp/rxn, 100 cp/rxn, 125 cp/rxn, 150 cp/rxn 175 cp/rxn, 200 cp/rxn, 225 cp/rxn 250 cp/rxn, 250 cp/rxn, 275 cp/rxn, 300 cp/rxn, 325 cp/rxn, 350 cp/rxn, 375 cp/rxn, 400 cp/rxn, or 500 cp/rxn. In some embodiments, the method achieves a limit of detection for determining the presence of a target nucleic acid from Chlamydia trachomatis and one or more additional target nucleic acid from Neisseria gonorrhoeae and/or Trichomonas vaginalis of a least about 0.5 copies per reaction (cp/rxn), 0.6 cp/rxn, 0.7 cp/rxn, 0.8 cp/rxn, 0.9 cp/rxn, 1.0 cp/rxn, 1.1 cp/rxn, 1.2 cp/rxn, 1.3 cp/rxn, 1.4 cp/rxn, 1.5 cp/rxn, 1.6 cp/rxn, 1.7 cp/rxn, 1.8 cp/rxn, 1.9 cp/rxn, 2.0 cp/rxn, 3 cp/rxn, 4 cp/rxn, 5 cp/rxn, 6 cp/rxn, 7 cp/rxn, 8, cp/rxn, 9 cp/rxn, 10 cp/rxn, 11 cp/rxn, 12 cp/rxn, 13 cp/rxn, 14 cp/rxn, 15 cp/rxn, 16 cp/rxn, 17 cp/rxn, 18 cp/rxn, 19 cp/rxn, 20 cp/rxn, 25 cp/rxn, 30 cp/rxn, 35 cp/rxn, 40 cp/rxn, 45 cp/rxn, 50 cp/rxn, 55 cp/rxn, 60 cp/rxn, 65 cp/rxn, 70 cp/rxn 75 cp/rxn, 80 cp/rxn, 85 cp/rxn, 90 cp/rxn, 95 cp/rxn, 100 cp/rxn, 125 cp/rxn, 150 cp/rxn 175 cp/rxn, 200 cp/rxn, 225 cp/rxn 250 cp/rxn, 250 cp/rxn, 275 cp/rxn, 300 cp/rxn, 325 cp/rxn, 350 cp/rxn, 375 cp/rxn, 400 cp/rxn, or 500 cp/rxn for each of the target nucleic acid sequences being detected.

In some embodiments, the method achieves a limit of detection for determining the presence of a target nucleic acid sequence from Neisseria gonorrhoeae of at least about 0.5 copies per reaction (cp/rxn), 0.6 cp/rxn, 0.7 cp/rxn, 0.8 cp/rxn, 0.9 cp/rxn, 1.0 cp/rxn, 1.1 cp/rxn, 1.2 cp/rxn, 1.3 cp/rxn, 1.4 cp/rxn, 1.5 cp/rxn, 1.6 cp/rxn, 1.7 cp/rxn, 1.8 cp/rxn, 1.9 cp/rxn, 2.0 cp/rxn, 3 cp/rxn, 4 cp/rxn, 5 cp/rxn, 6 cp/rxn, 7 cp/rxn, 8, cp/rxn, 9 cp/rxn, 10 cp/rxn, 11 cp/rxn, 12 cp/rxn, 13 cp/rxn, 14 cp/rxn, 15 cp/rxn, 16 cp/rxn, 17 cp/rxn, 18 cp/rxn, 19 cp/rxn, 20 cp/rxn, 25 cp/rxn, 30 cp/rxn, 35 cp/rxn, 40 cp/rxn, 45 cp/rxn, 50 cp/rxn, 55 cp/rxn, 60 cp/rxn, 65 cp/rxn, 70 cp/rxn 75 cp/rxn, 80 cp/rxn, 85 cp/rxn, 90 cp/rxn, 95 cp/rxn, 100 cp/rxn, 125 cp/rxn, 150 cp/rxn 175 cp/rxn, 200 cp/rxn, 225 cp/rxn 250 cp/rxn, 250 cp/rxn, 275 cp/rxn, 300 cp/rxn, 325 cp/rxn, 350 cp/rxn, 375 cp/rxn, 400 cp/rxn, or 500 cp/rxn. In some embodiments, the method achieves a limit of detection for determining the presence of a target nucleic acid from Neisseria gonorrhoeae and one or more additional target nucleic acid from Chlamydia trachomatis and/or Trichomonas vaginalis of a least about 0.5 copies per reaction (cp/rxn), 0.6 cp/rxn, 0.7 cp/rxn, 0.8 cp/rxn, 0.9 cp/rxn, 1.0 cp/rxn, 1.1 cp/rxn, 1.2 cp/rxn, 1.3 cp/rxn, 1.4 cp/rxn, 1.5 cp/rxn, 1.6 cp/rxn, 1.7 cp/rxn, 1.8 cp/rxn, 1.9 cp/rxn, 2.0 cp/rxn, 3 cp/rxn, 4 cp/rxn, 5 cp/rxn, 6 cp/rxn, 7 cp/rxn, 8, cp/rxn, 9 cp/rxn, 10 cp/rxn, 11 cp/rxn, 12 cp/rxn, 13 cp/rxn, 14 cp/rxn, 15 cp/rxn, 16 cp/rxn, 17 cp/rxn, 18 cp/rxn, 19 cp/rxn, 20 cp/rxn, 25 cp/rxn, 30 cp/rxn, 35 cp/rxn, 40 cp/rxn, 45 cp/rxn, 50 cp/rxn, 55 cp/rxn, 60 cp/rxn, 65 cp/rxn, 70 cp/rxn 75 cp/rxn, 80 cp/rxn, 85 cp/rxn, 90 cp/rxn, 95 cp/rxn, 100 cp/rxn, 125 cp/rxn, 150 cp/rxn 175 cp/rxn, 200 cp/rxn, 225 cp/rxn 250 cp/rxn, 250 cp/rxn, 275 cp/rxn, 300 cp/rxn, 325 cp/rxn, 350 cp/rxn, 375 cp/rxn, 400 cp/rxn, or 500 cp/rxn for each of the target nucleic acid sequences being detected.

In some embodiments, the method achieves a limit of detection for determining the presence of a target nucleic acid sequence from Trichomonas vaginalis of at least about 0.5 copies per reaction (cp/rxn), 0.6 cp/rxn, 0.7 cp/rxn, 0.8 cp/rxn, 0.9 cp/rxn, 1.0 cp/rxn, 1.1 cp/rxn, 1.2 cp/rxn, 1.3 cp/rxn, 1.4 cp/rxn, 1.5 cp/rxn, 1.6 cp/rxn, 1.7 cp/rxn, 1.8 cp/rxn, 1.9 cp/rxn, 2.0 cp/rxn, 3 cp/rxn, 4 cp/rxn, 5 cp/rxn, 6 cp/rxn, 7 cp/rxn, 8, cp/rxn, 9 cp/rxn, 10 cp/rxn, 11 cp/rxn, 12 cp/rxn, 13 cp/rxn, 14 cp/rxn, 15 cp/rxn, 16 cp/rxn, 17 cp/rxn, 18 cp/rxn, 19 cp/rxn, 20 cp/rxn, 25 cp/rxn, 30 cp/rxn, 35 cp/rxn, 40 cp/rxn, 45 cp/rxn, 50 cp/rxn, 55 cp/rxn, 60 cp/rxn, 65 cp/rxn, 70 cp/rxn 75 cp/rxn, 80 cp/rxn, 85 cp/rxn, 90 cp/rxn, 95 cp/rxn, 100 cp/rxn, 125 cp/rxn, 150 cp/rxn 175 cp/rxn, 200 cp/rxn, 225 cp/rxn 250 cp/rxn, 250 cp/rxn, 275 cp/rxn, 300 cp/rxn, 325 cp/rxn, 350 cp/rxn, 375 cp/rxn, 400 cp/rxn, or 500 cp/rxn. In some embodiments, the method achieves a limit of detection for determining the presence of a target nucleic acid from Trichomonas vaginalis and one or more additional target nucleic acid from Chlamydia trachomatis and/or Neisseria gonorrhoeae of a least about 0.5 copies per reaction (cp/rxn), 0.6 cp/rxn, 0.7 cp/rxn, 0.8 cp/rxn, 0.9 cp/rxn, 1.0 cp/rxn, 1.1 cp/rxn, 1.2 cp/rxn, 1.3 cp/rxn, 1.4 cp/rxn, 1.5 cp/rxn, 1.6 cp/rxn, 1.7 cp/rxn, 1.8 cp/rxn, 1.9 cp/rxn, 2.0 cp/rxn, 3 cp/rxn, 4 cp/rxn, 5 cp/rxn, 6 cp/rxn, 7 cp/rxn, 8, cp/rxn, 9 cp/rxn, 10 cp/rxn, 11 cp/rxn, 12 cp/rxn, 13 cp/rxn, 14 cp/rxn, 15 cp/rxn, 16 cp/rxn, 17 cp/rxn, 18 cp/rxn, 19 cp/rxn, 20 cp/rxn, 25 cp/rxn, 30 cp/rxn, 35 cp/rxn, 40 cp/rxn, 45 cp/rxn, 50 cp/rxn, 55 cp/rxn, 60 cp/rxn, 65 cp/rxn, 70 cp/rxn 75 cp/rxn, 80 cp/rxn, 85 cp/rxn, 90 cp/rxn, 95 cp/rxn, 100 cp/rxn, 125 cp/rxn, 150 cp/rxn 175 cp/rxn, 200 cp/rxn, 225 cp/rxn 250 cp/rxn, 250 cp/rxn, 275 cp/rxn, 300 cp/rxn, 325 cp/rxn, 350 cp/rxn, 375 cp/rxn, 400 cp/rxn, or 500 cp/rxn for each of the target nucleic acid sequences being detected.

### System

Aspects of the present disclosure include a system for detecting whether at least one sexual health condition is present in a user.

In some embodiments, the system includes an at-home kit for the user to collect a sample, the kit comprising: a sample collection device for receiving the sample collected by a user at home or at a clinic; a storage container storing a preservation buffer designed for receiving samples collected at home; and a labeled shipping container for shipping the storage container holding the preservation buffer and the sample to a laboratory for analysis.

In some embodiments, the system further includes a laboratory analysis kit comprising a nucleic acid extraction kit; an amplification reaction mixture; and primers, comprising at least two selected from: a first pair of primers that bind specifically to a target nucleic acid sequence from Chlamydia trachomatis where present in the sample, a second pair of primers that bind specifically to a target nucleic acid sequence from Neisseria gonorrhoeae, where present in the sample, and a third pair of primers that bind specifically to a target nucleic acid sequence from Trichomonas vaginalis where present in the sample.

In some embodiments, the system further includes a computer-readable medium storing code for that when executed by a processor cause the processor to perform the following steps: receiving and displaying results of analysis with the laboratory analysis kit to the user (e.g. “Your Test Results” of FIG. 3), the results providing information about whether or not the user has the sexual health condition. In some embodiments, the storing code executed by the processor further causes the processor to initiate communication and/or telemedicine (e.g., “Register a kit” of FIG. 3) with a healthcare provider via an online portal to provide patient care (FIG. 3). In some embodiments, the communication can be for purposes of running further tests, displaying results, communication results, providing telehealth visits, prescriptions and/or medications.

In some embodiments, the sexual health condition is an infection associated with Chlamydia trachomatis. In some embodiments, the Chlamydia trachomatis infections are associated with Chlamydia trachomatis strains selected from Serovar D, Serovar E, Serovar F, Serovar G, Serovar H, Serovar I, Serovar J, Serovar K and Serovar LGV.

In some embodiments, the sexual health condition is an infection associated with Neisseria gonorrhoeae. In certain embodiments, the Neisseria gonorrhoeae infections are associated with Neisseria gonorrhoeae strains selected from AR-0166, AR-0168, AR-0205, AR-0206, AR-0207, AR-0208, AR-0209, AR-0210, AR-0211, AR-0212, AR-0213, AR-0214, CDC Ng-98, and CDC Ng-116.

In some embodiments, the sexual health condition is an infection associated with Trichomonas vaginalis. In certain embodiments, the Trichomonas vaginalis infections are associated with Trichomonas vaginalis strains selected from 165307-1, JH 32A #2, 11769, NYH 286, RU 357, MT87, HsD:NIH, TVC, and NYH 286.

In some embodiments, the storage buffer comprises phosphate buffered saline, Tris-buffered saline, HEPES buffered saline, a reducing agent, sodium hydroxide, Triton X-100, and octyl glucoside.

In some embodiments, the amplification reaction mixture comprises a polymerase comprising 5′ nuclease activity.

In some embodiments, the first pair of primers, the second pair of primers, and the third pair of primers are within a single container.

In some embodiments, the amplification reaction mixture is within the single container. In some embodiments, the amplification reaction mixture, the DNA template suspected to comprise a target nucleic acid sequence within the sample, and the primers are within the single container. In some embodiments, the amplification reaction mixture, the first pair of primers, the second pair of primers, the third pair of primers, and a plurality of target nucleic acid sequences from the sample (e.g., DNA template) are within the single container.

In some embodiments, the first pair of primers, the second pair of primers, and the third pair of primers, each optionally include a probe, wherein the probe binds specifically to the respective target nucleic acid sequence.

In some embodiments, the first forward primer, the second forward primer, the third forward primer, or each of the first, second, and third forward primers comprises a concentration ranging from 0-05-1 uM. In some embodiments, the first reverse primer, the second reverse primer, the third reverse primer, or each of the first, second, and third reverse primers comprises a concentration ranging from 0-05-1 uM. In some embodiments, each probe comprises a concentration ranging from 0-05-1 uM.

In some embodiments, the first pair of primers comprise a forward primer having a nucleotide sequence selected from SEQ ID NO: 1-19, a reverse primer having a nucleotide sequence selected from SEQ ID NO: 20-35, and optionally, a probe having a nucleotide sequence selected from SEQ ID NO: 36-47. In some embodiments, the second pair of primers comprise a forward primer having a nucleotide sequence selected from SEQ iD NOs: 48-59, a reverse primer having a nucleotide sequence selected from SEQ ID NO: 60-70, and optionally, a probe having a nucleotide sequence selected from SEQ iD NOs: 71-84. In some embodiments, the third pair of primers comprise a forward primer having a nucleotide sequence selected from SEQ ID NO: 85-95, a reverse primer having a nucleotide sequence selected from SEQ ID NO: 96-104, and optionally, a probe having a nucleotide sequence selected from SEQ ID NO: 105-115.

In some embodiments, the first pair of primers, the second pair of primers, the third pair of primers, the optional probes, or a combination thereof include detectably labeled primers and/or probes, wherein the probe(s) bind specifically to the respective target nucleic acid sequence. In some embodiments, the detectably labeled probes comprise a dual-labeled probe.

In some embodiments, the at-home kit further comprises a sample collection tool. In some embodiments, the results of the analysis is displayed through an online portal.

### Telehealth and Platform for Consumer (User)

Aspects of the present disclosure include a non-transitory computer-readable storage medium storing executable computer program instructions. Such instructions comprise code for carrying out the steps for the methods of the present disclosure, carrying out the steps of the system of the present disclosure, and/or providing a user interface between the patient (e.g., consumer, subject, and the like) and the platform.

The platform system for providing communication to and from a user (e.g. patient) for registering a kit, providing status updates of a test result, and providing personalized telehealth options for users, is described herein.

The platform system receives registration information about the kit of the present disclosure that the user has purchased, or a clinician has recommended for purchase. Before or after the kit has been used to provide the user sample to test for one or more sexual health conditions, the platform system allows the user to self-register the kit using a user interface for the platform system. The platform system also receives registration information from the user. The platform system can also provide status updates of the sample that is shipped to the laboratory for processing. Such status updates can include once the sample has been received by the laboratory, status of whether the sample has been processed, and/or whether the test results are available to the user. Once the test results are processed and status of the user's test result is available, the platform system generates status updates based on the changes in status of the test results. Once the user receives the test result, the user can decide whether to seek additional personalized telehealth advice from the platform system. For example, if a user tests positive for one or more sexual health conditions, the user can fill out a patient questionnaire to seek additional information about the positive test result and next steps, such as, but not limited to, setting up a telehealth consult with a healthcare provider or clinician for explanation of the test result and additional medical options, and/or order a prescription for treating one or more sexual health conditions. As used herein, the term “positive result” or “positive test result”, can be described as a “abnormal” test or “abnormal test result”. For example, if the patient receives an abnormal test result, the patient is considered to have one or more sexual health conditions for which the patient is being tested for. For a positive or abnormal result, the patient can: download the test result, share the result with their healthcare provider, use the telehealth platform to get a consult and/or a prescription, and/or find a local clinical or a primary care provider (PCP).

### Platform Overview

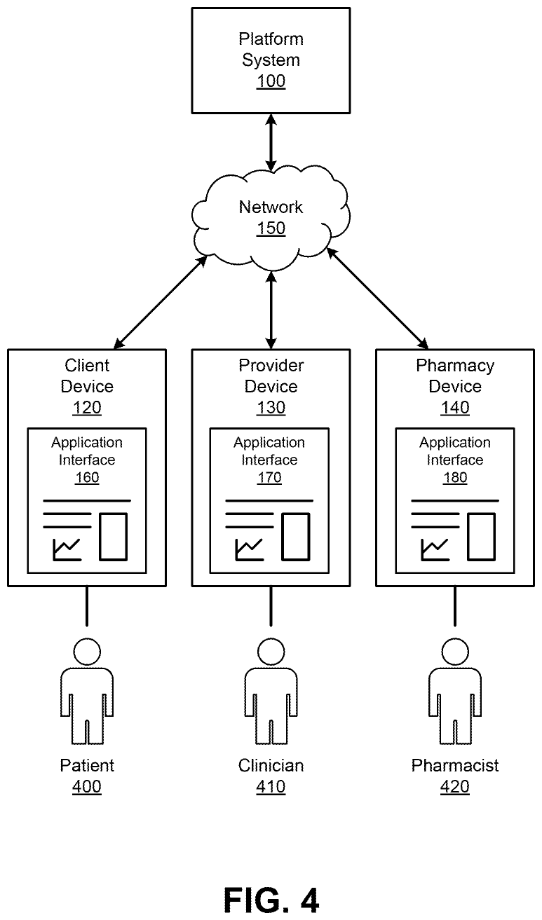

FIG. 4 provides a system environment for a platform system 100 for providing telehealth medicine for the user according to one embodiment. The environment includes the platform system 100, a client device 120 and associated application interface 160, a healthcare provider device 130 and associated application interface 170, a pharmacy device 140 and associated application interface 180, and a network 150. However, in other embodiments, the system 100 may include different and/or additional components.

For example, the client device 120 is a computing device operated by a user 400 to interact with the platform system. Similarly, the provider device 130 is a computing device operated by a provider, such as a clinician 410. In some cases, a single provider may have more than one device that interacts with the platform system 100, and a client device 120 may have more than one device that interacts with the platform system 100. In some embodiments, the system does not include a provider device or a pharmacy device. In some embodiments, the system includes a provider device 120. In some embodiments, the system includes a pharmacy device 130. In some embodiments, the system includes a provider device 120 and a pharmacy device 130.

The client device 120 is a computing device with data processing and data communication capabilities that is capable of receiving inputs from a client. The client can include the user or patient (used interchangeably herein as “subject”). In addition to data processing, the client device may include functionality that allows the device 120 to record speech responses articulated by a patient operating the device (e.g., a microphone), and to graphically present data to a patient (e.g., a graphics display). Examples of the client device 120 include desktop computers, laptop computers, portable computers, google home, amazon echo, mobile devices, and the like. The client device 120 may present information generated by the platform system 100 via a mobile application configured to display and record patient responses. For example, through a software application interface 160, the patient may receive a kit registration questionnaire, a patient questionnaire, a status update regarding a test result, or telehealth recommendations and input based on the patient's test result.

Application 160 provides a user interface (herein referred to as a “patient dashboard”) that is displaced on a screen of the client device 120 and allows the patient to input commands to control the operation of the application 160. The patient dashboard enables patients to track and manage status of a test result, status of a prescription and/or input or recommendation on treatment options if a patient testes positive for a sexual health condition. For example, the dashboard allows patients to receive recommendation notifications, exchange messages about treatment with a clinician, and so on. The application 160 may be coded as a web page, series of web pages, or content otherwise coded to render within an internet browser. The application 160 may also be coded as a proprietary application configured to operate on the native operating system of the client device 120. In addition to providing the dashboard, application 160 sends information about the patient (e.g., registration information, eligibility information, medical history, responses to patient questionnaire, etc.) through the network 150. Patient data sent through the network 150 is received by the platform system 100 where it is analyzed and processed for storage and retrieval in conjunction with a database.

Similarly, the healthcare provider device 130 is a computing device with data processing and data communication capabilities that is capable of receiving input from a provider. In some embodiments, the provider device 130 is configured to review a patient's medical history or medically relevant data (i.e., a display screen) that is provided from a client device 120, to the platform. The above description of the functionality of the client device 120 also can apply to the provider device 130. The provider device 130 can be a personal device (e.g., phone, tablet) of the provider, a medical institution computer (e.g., a desktop computer of a hospital or medical facility), etc. In addition, the provider device 130 can include a device that sits within the provider office such that the patient can interact with the device inside the office. In such implementations, the provider device is a customized device with audio and/or video capabilities (e.g., a microphone for recording, a display screen for text and/or video, an interactive user interface, a network interface, etc.). The provider device 130 may also present information to medical providers or healthcare organizations via a mobile application similar to the application described with reference to client device 120.

Application 170 provides a user interface (herein referred to as a “provider dashboard”) that is displayed on a screen of the provider device 130 and allows a medical provider or trained professional/coach to input commands to control the operation of the application 170. The provider dashboard enables providers to track and manage patient data provided by platform that came from the client device 120. The application 170 may be coded as a web page, series of web pages, or content otherwise coded to render within an internet browser. The application 170 may also be coded as a proprietary application configured to operate on the native operating system of the client device 120.

Similarly, the pharmacy device 140 is a computing device with data processing and data communication capabilities that is capable of receiving input from the platform system 100. The pharmacy device can be used by a pharmacy employee, such as a pharmacy technician or a pharmacist 420 as shown in FIG. 4. In some embodiments, the pharmacy device 140 is configured to review a prescription request (i.e., a display screen) that is provided from the platform system 100. The above description of the functionality of the client device 120 also can apply to the pharmacy device 140. The pharmacy device 140 can be a personal device (e.g., phone, tablet) of the pharmacy, pharmacist, technician, a computer (e.g., a desktop computer of a hospital or medical facility or pharmacy), etc. In such implementations, the pharmacy device is a customized device with audio and/or video capabilities (e.g., a microphone for recording, a display screen for text and/or video, an interactive user interface, a network interface, etc.).

Application 180 provides a user interface (herein referred to as a “pharmacy dashboard”) that is displayed on a screen of the pharmacy device 140 and allows a medical provider or trained professional/coach to input commands to control the operation of the application 180. The provider dashboard enables providers to track and manage patient prescriptions provided by platform that came from the platform system 100. The application 180 may be coded as a web page, series of web pages, or content otherwise coded to render within an internet browser. The application 180 may also be coded as a proprietary application configured to operate on the native operating system of the client device 120.

The components of the platform system, such as the client device 120, the provider device 130, and the pharmacy device all interact with the network 150, which relays all information from each of the devices to the platform system 100. Thus, interactions between the client device 120, the provider device 130, and the pharmacy device 140, and the platform system 100 are typically performed via the network 150, which enables communication between the client device 120, the provider device 130, the pharmacy device 140, and the communication platform system 100. In one embodiment, the network 150 uses standard communication technologies and/or protocols including, but not limited to, links using technologies such as Ethernet, 802.11, worldwide interoperability for microwave access (WiMAX), 3G, 4G, LTE, digital subscriber line (DSL), asynchronous transfer mode (ATM), InfiniBand, and PCI Express Advanced Switching. The network 150 may also utilize dedicated, custom, or private communication links. The network 150 may comprise any combination of local area and/or wide area networks, using both wired and wireless communication systems.

The devices described in the system (e.g., client device, provider device, pharmacy device) can each include physical components of a computer that may be used as a client device, provider device, pharmacy device, and/or server. Such components can include a chipset coupled to a processor, a volatile memory, a network adapter, and input/output (I/O) device, a storage device representing a non-volatile memory, and a display. In some embodiments, the memory is coupled directly to the processor instead of a chipset. In some embodiments, memory includes high-speed random access memory (RAM), such as DRAM, SRAM, DDR RAM or other random access solid state memory devices.

A storage device is any non-transitory computer-readable storage medium, such as a hard drive, compact disk read-only memory (CD-ROM), DVD, or a solid-state memory device. The memory holds instructions and data used by the processor. The (I/O) device may be a touch input surface (capacitive or otherwise), a mouse, track ball, or other type of pointing device, a keyboard, or another form of input device. The display displays images and other information for the computer. The network adapter couples the computer to the network 150. As is known in the art, a computer can have different and/or other components. In addition, the computer can lack certain illustrated components. In one embodiment, a computer acting as server may lack a dedicated I/O device, and/or display 218. Moreover, the storage device can be local and/or remote from the computer (such as embodied within a storage area network (SAN)), and, in one embodiment, the storage device is not a CD-ROM device or a DVD device.

Generally, the exact physical components used in a client device will vary in size, power requirements, and performance from those used in the application server and the database server. For example, client devices, which will often be home computers, tablet computers, laptop computers, or smart phones, will include relatively small storage capacities and processing power, but will include input devices and displays. These components are suitable for user input of data and receipt, display, and interaction with notifications provided by the application server. In contrast, the application server may include many physically separate, locally networked computers each having a significant amount of processing power for carrying out the asthma risk analyses introduced above. In one embodiment, the processing power of the application server provided by a service such as Amazon Web Services™. Also in contrast, the database server 140 may include many, physically separate computers each having a significant amount of persistent storage capacity for storing the data associated with the application server.

As is known in the art, the computer is adapted to execute computer program modules for providing functionality described herein. A module can be implemented in hardware, firmware, and/or software. In one embodiment, program modules are stored on the storage device, loaded into the memory, and executed by the processor.

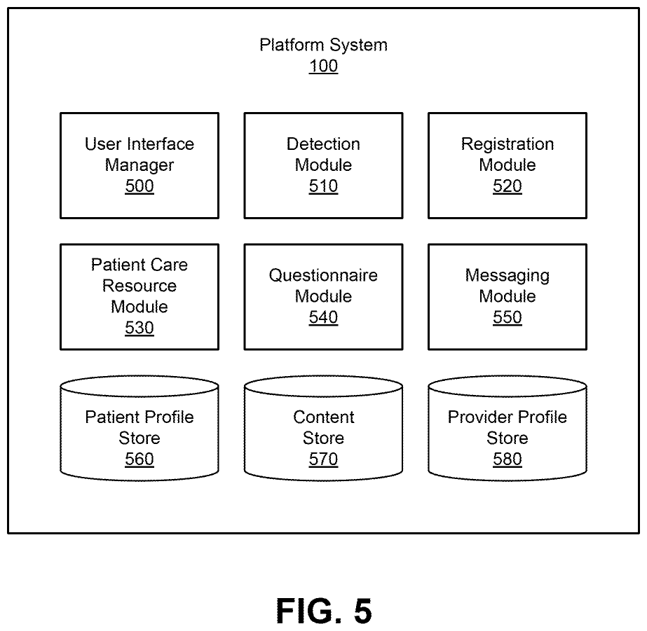

FIG. 5 of the present disclosure shows a block diagram of the system architecture of the platform system 100 within the platform of FIG. 1, according to one embodiment. For example, the module can include a user interface manager 500, a detection module 510, registration module 520, patient care resource module 530, questionnaire module 240, and a messaging module 550. Other modules can also be included within the platform, such as content stores (e.g., patient profile store 560, content store 570, or provider profile store 580). In other embodiments, the platform system 100 may include additional, fewer, and/or different modules for various applications.

Conventional components such as network interfaces, security mechanisms, load balancers, failover servers, management and network operations consoles, and the like are not shown so as to not obscure the details of the system 100. Also, it is noted that the modules may be embodied as hardware, software (which may include firmware), or any combination thereof. For software, it may include program code or code segments, e.g., for a native application on a mobile client device. Software is comprised of one or more instructions storable in a computer readable storage medium, e.g., a memory or disk, and executable by a processor.

The user interface manager 500 manages communication with the user devices in communication with the platform system 100. The user interface manager 500 may generate and transmit a user interface to the user devices. The user interface may include a login portal to access secure data. In some embodiments, the user interface may be modified based on a type of user accessing the platform system 100, e.g., patient, provider, or pharmacist. The user interface is configured to display information and/or to receive input from the user devices.

The detection module 510 is configured to detect one or more sexual health conditions based on PCR results, e.g., received from a PCR thermocycler. In one or more embodiments, the platform system 100 communicates with the PCR thermocycler, which outputs the quantification of the target nucleic acid sequences, e.g., amplification curves as detected by the PCR thermocycler. The detection module 510 may assign amplification curves to each target nucleic acid sequence, e.g., based on the characteristics of the fluorescence signals. The detection module 510 may determine presence of the target nucleic acid sequence based on the quantification, e.g., if there is a minimum threshold fluorescence intensity, indicating more than a baseline concentration of the nucleic acid sequence present in the processed sample.

The registration module 520 handles registration. The registration module 520 may request user information from the user device. In some embodiments, the registration module 520 also requests a Sample ID of the sample collected for a patient. The registration module 520 may link a user profile of the patient to the Sample ID. The registration module 520 may also aid in creation of a user profile, e.g., for new users.

The patient care resource module 530 monitors the patient's condition and may provide any resources for patient care. In some embodiments, the patient care resource module 530 may maintain a treatment plan for the patient, e.g., as determined by the patient with the patient's healthcare provider. The patient care resource module 530 may monitor adherence to the treatment plan, e.g., via reminders to the patient. The patient care resource module 530 may also provide additional content to the patient to aid with patient care.

The questionnaire module 540 creates the questionnaire for the users. The questionnaire module 540 generates the questionnaire based on previously collected information, e.g., to avoid collection of redundant information.

The messaging module 550 creates messages to the user devices. In some embodiments, the messaging module 550 may create messages automatically, e.g., based on analytical results by the platform system 100 or any other results. The messaging module 550 may also send messages between users. For example, if the patient sends a message to the patient's healthcare provider, the messaging module 550 may send that message to the user device of the healthcare provider.

The stores store data used by the platform system 100. For example, the patient profile store 560 may store patient profiles of the various patients. Each patient profile may include information on the patient, including but not limited to: personal identifying information, medical history, demographic information, diagnoses, diagnostic results, past sample results, treatment plan(s), associated provider information, associated pharmacy information, etc. The content store 570 may store information for various users. For example, the content store 570 may include information on various treatment plans, helpful tips, advice, articles, etc. The provider profile store 580 stores provider profiles. Each provider profile may include information on the provider, including but not limited to: personal identifying information, credentials, associated patients, etc.

### Non-Transitory Computer Readable Storage Medium

Aspects of the present disclosure include a non-transitory computer-readable storage medium storing executable computer program instructions, the computer program instructions comprising code for a series of steps. These series of steps, when executed by one or more processors, cause one or more processors to perform the steps described herein.

In some embodiments, the program instructions comprise code for: (a) receiving, from a client device, registration information about the patient and information about a kit registered to the patient for detecting status of one or more sexual health conditions; (b) providing, to the client device, status information indicating whether or not the patient has tested positive or negative for one or more sexual health conditions; wherein, if the status information indicates that the patient has tested positive (e.g., receives an abnormal test rest result) for one or more sexual health conditions, the computer program instructions further comprise code for: providing, to the client device, a patient questionnaire; providing, to the client device, a plurality of options selected from: a telehealth consult with a clinician; an order prescription request; and a combination thereof; and receiving, from the client device, input related to the selection options; and providing, to the client device, a treatment care option based on input received related to the selected option.

For example, if the patient receives an abnormal test result, the patient is considered to have one or more sexual health conditions for which the patient is being tested for. For a positive or abnormal result, the patient can: download the test result, share the result with their healthcare provider, use the telehealth platform to get a consult and/or a prescription, and/or find a local clinical or a primary care provider (PCP).

### Registration Information

Registration information that the client device provides to the platform can include information about the kit for registering the kit, a patient's medical history, screening questions, and the like. For example, in some embodiments, the registration information about the patient comprises providing a registration process to the client device, the registration process comprising a registration questionnaire to the patient to answer screening questions and indicate information about the patient. In some embodiments, the registration questionnaire about the patient comprises instructions to the patient to provide patient payment information. In some embodiments, the registration questionnaire about the patient comprises instructions to the patient to provide patient healthcare insurance information.

Additional registration information can include information about the patient's sample. For example, the registration questionnaire about the kit can include instructions to the patient to provide a sample collection time.

In some embodiments, the registration questionnaire about the kit comprises instructions to the patient to provide the sample ID, test name, sample collection date, and sample collection time.

In certain embodiments, patient history about the patient comprises instructions to the patient to provide whether or not the patient has tested positive for one or more sexual health conditions. In certain embodiments, history about the patient comprises instructions to the patient to provide information about the patient's sexual history.

In some embodiments, the registration questionnaire about the patient further comprises providing the information about the patient to the healthcare provider device, wherein the healthcare provider device 130 is used by a healthcare provider. In some embodiments, the registration questionnaire about the patient further comprises providing the information about the patient to the platform, where the platform provides the information about the patient to the healthcare provider device 130.

### Status of Test Result

Once the sample is sent to a laboratory (e.g., in house laboratory or a third party laboratory), the platform can provide a status of where the sample is in processing (e.g., sample has been received by the facility, sample is being processed to determine whether the patient is positive or negative for one or more sexual health conditions, etc.). Therefore, the platform can provide status of the test result of the sample to the client device.

Once the test result is ready for sending out to the patient, the platform provides, to the client device, status of whether the patient has tested positive or negative for one or more sexual health conditions. The patient can then decide whether the patient wants to seek further medical attention via the telehealth platform (e.g., if a patient tests positive for one or more sexual health conditions).

### Telehealth Options Following Test Result

In some embodiments, once the test result is ready for sending out to the patient, the platform provides, to the client device, the test or laboratory result. In certain embodiments, the patient can download the test result on the client device. For example, if the patient receives an abnormal test result, the patient can: download the test result from the platform (or, in other words, the platform provides the test result to the patient as a downloadable test result), share the test result with their healthcare provider, use the telehealth platform to get a consult and/or a prescription, and/or find a local clinical or a primary care provider (PCP).

If the patient wants to seek further medical attention, the patient can fill out a patient questionnaire prompted by the platform. For example, if the patient tests positive for one or more sexual health conditions, the computer program instructions further comprise code for providing, to the client device, a patient questionnaire.

The patient can then fill out and respond to the patient questionnaire on the client device, which can then be sent to back to the platform. The patient questionnaire can include eligibility information about the patient to determine whether additional medical attention or a prescription is needed for treating the positive test result of one or more sexual health conditions. In some embodiments, eligibility information comprises preferences and/or symptom information from the patient. In some embodiments, eligibility information comprises treatment preferences from the patient. In some embodiments, eligibility information about the patient comprises medical history about the patient. In some embodiments, eligibility information about the patient comprises instructions to the patient to provide the patient's date of birth and address. In some embodiments, eligibility information about the patient comprises information about one or more of the following: whether or not the patient is pregnant; whether or not the patient is breastfeeding; whether or not the patient is allergic to one or more medications; whether or not the patient is taking antibiotics; whether or not the patient is taking birth control; and a list of current medications. In some embodiments, eligibility information comprises identification information about the patient.

Once the patient fills out the patient questionnaire and sends it back to the platform, the patient can provide, to the client device, a plurality of options for the patient in terms of next steps. The platform can provide educational reading materials, a telehealth consult with a clinician, an order prescription request, or a combination thereof.

If the patient wants to seek further medical information, the patient can request a consult with a telehealth clinician. In some embodiments, the telehealth clinician is selected from: a nurse practitioner, a physician's assistant, a trained medical technician, and a physician. The clinician can then provide explanation of the test result, and potential treatment options or next steps based on responses to the patient questionnaire and come up with a treatment plan. For example, if the patient is pregnant, during the telehealth consult with the clinician, the clinician will provide alternative medication options to the patient or route the patient to a primary care physician. In some embodiments, if the patient is breastfeeding, during the telehealth consult with the clinician, the clinician will provide alternative medication options to the patient or route the patient to a physician. In some embodiments, if the patient has one or more allergies to one or more medications, during the telehealth consult with the clinician, the clinician will provide alternative medication options to the patient or route the patient to a physician. In some embodiments, if the patient is taking birth control, during the telehealth consult with the clinician, the clinician will provide alternative medication options to the patient or route the patient to a primary care physician. In some embodiments, if the patient is taking one or more medications, during the telehealth consult with the clinician, the clinician will provide alternative medication options to the patient or route the patient to a primary care physician.

If, after the patient fills out the patient questionnaire and is received by the platform and reviewed, the patient can simply request a prescription for treatment of the sexual health condition with or without a telehealth consult with a clinician. In some embodiments, the client can provide, on the client device, shipping information of the prescription to the patient's location to the platform or the pharmacy device. In some embodiments, the prescription details comprise pickup information of the prescription from a local pharmacy.

For example, if the patient wants to request a prescription, the client device can send a prescription request to the platform. In some embodiments, if the order prescription request is received from the client device, the computer program instructions comprise code for providing, to a client device, approval or denial of the order prescription request. Based on the information provided to the platform (e.g., via patient questionnaire that the patient fills out and sends via client device to the platform), the platform can provide approval or denial of the order prescription request. In some embodiments, the platform sends the order prescription request to the provider device 130 for the clinician to determine eligibility of the patient for receiving an approved prescription. In some embodiments, the platform does not send the order prescription request to the provider device 130 to determine eligibility of the patient for receiving an approved prescription.

In some embodiments, if the order prescription request is approved: the computer program instructions comprise code providing, to the client device, a prescription order of the medication and instructions for use of the medication to the patient, prescription order delivery options. For example, once the prescription request is approved, the client, on the application interface, can find a local pharmacy affiliated with the platform for which the client can order the prescription and either get the prescription delivered or pick up the prescription from the affiliated local pharmacy. Additionally, the platform or pharmacy device may send/provide the client device a payment request for the prescription.

### Processing the Sample

In some embodiments, once the patient has sent the kit containing the sample to the laboratory facility and registers the kit, the computer program further comprises code for receiving, PCR results related to the sample ID of the kit. In some embodiments, before step (b) of the code, the code further comprises instructions for operating one or more laboratory devices for receiving one or more samples acquired directly from the patient or by a clinician; amplifying a target nucleic acid sequence of a plurality of target nucleic acid sequences from the sample by PCR amplification using primers and an amplification reaction mixture, wherein the primers comprise at least two selected from: (i) a first pair of primers that bind specifically to a target nucleic acid sequence from Chlamydia trachomatis, (ii) a second pair of primers that bind specifically to a target nucleic acid sequence from Neisseria gonorrhoeae, (iii) a third pair of primers that bind specifically to a target nucleic acid sequence from Trichomonas vaginalis, or (iv) a combination thereof; detecting the presence or absence of the PCR amplification product, thereby determining status of at least one sexual health condition; providing test results to the patient via an online portal, and optionally communicating with a healthcare provider using the online portal.

### A Computer Implemented Method

Another aspect of the present disclosure comprises a computer implemented method for providing telehealth medicine and status of one or more sexual health conditions to a patient, the method comprising: receiving, from a client device, registration information about the patient and information about a kit registered to the patient for detecting status of one or more sexual health conditions; providing, to the client device, status information indicating whether or not the patient has tested positive or negative for one or more sexual health conditions; wherein, if the status information indicates that the patient has tested positive for one or more sexual health conditions, the method further comprises: providing, to the client device, a patient questionnaire; providing, to the client device, a plurality of options selected from: a telehealth consult with a clinician; an order prescription request; and a combination thereof; and receiving, from the client device, input related to the selection options; and providing, to the client device, a treatment care option based on input received related to the selected option.

The steps of the computer non-transitory computer readable storage medium described in the present disclosure can be applied to the computer implemented method.

The foregoing description of the embodiments has been presented for the purpose of illustration; it is not intended to be exhaustive or to limit the patent rights to the precise forms disclosed. Persons skilled in the relevant art can appreciate that many modifications and variations are possible in light of the above disclosure.

Any of the steps, operations, or processes described herein may be performed or implemented with one or more hardware or software modules, alone or in combination with other devices.

Embodiments may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Finally, the language used in the specification has been principally selected for readability and instructional purposes, and it may not have been selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights, one implementation of which is set forth in the following claims.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); microliters (μl), s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

Materials include: an extraction buffer, a spin-column, a wash buffer, and an elution buffer.

### Sample Collection

First-catch urine was collected in a sterile, plastic, preservative-free specimen collection cup. One mL of urine was transferred with a transfer pipette into BD MAX™ UVE Sample Buffer Tube within 4 hours. Samples were transported to the laboratory under conditions of time and temperature ranges that had been determined to maintain the integrity and stability of the target nucleic acids within the sample.

Home sample collection for urine samples were validated following study guidelines as per FDA Home Specimen Collection Molecular Diagnostic Template. Sample stability studies were conducted in summer condition or winter condition for 134 hours (see Example 3). For each pathogen, inactivated cells were spiked in pools of diluent that included negative donor urine and BD™ MAX™ UVE Sample Buffer at 2×LoD (low positives, n=20) and 10×LoD (high positives, n=10). In addition, 10 negative samples were evaluated to monitor for false positives. The samples in the study were subjected to the temperature cycling profile for the indicated hours then extracted and assayed with the multiplex assay at the end of the experiment. In summary, specimens have passed established acceptance criteria and a stability claim of up to 5 days (134 hours) was validated (see Example 3).

### DNA Isolation Protocol

Zymo Research Quick-DNA™ Microprep Kit (Cat No. D3021) was used to extract DNA from samples. In particular, 200 μl of sample was used as an input material and DNA was eluted with 12 μl of DNA Elution Buffer. The Zymo Research Quick-DNA™ Microprep Kit was used according to the manufacturer's instructions, unless otherwise noted below. Prior to extracting DNA, a reducing agent (e.g., beta-mercaptoethanol) was added to genomic lysis buffer. DNA eluted in Elution Buffer was stored at room temperature or 4° C. till is used in qPCR reaction.

### Amplification Reaction

Amplification is performed in QuantStudio 3 PCR machine using in-house designed STD Primers and Probes, RNase P (RP) from IDT SARS-CoV-2 (2019-nCoV) CDC RUO Primers and Probes, and IDT PrimeTime® Gene Expression Master Mix (2×), as per manufacturer instructions. Nucleic acid is amplified in 20 μl reaction containing 4.5 μl of DNA template.

Mastermixes were prepared according to Table 11.

TABLE 11
Mastermixes.

| | STD mix | RP |
|---|---|---|
| PCR grade water | 5.26 μl | 4 μl |
| primers/probe mix specific for target | 0.24 μl | 1.5 μl |
| (CT, NG, and/or TV or RP) | | |
| 2 x PCR mix | 10 μl | 10 μl |
| DNA template | 4.5 μl | 4.5 μl |
| total sample volume | 20 μl | 20 μl |

Thermal cycler amplification profiles were set according to Table 12.

TABLE 12
Amplification profile

| | | | |
|---|---|---|---|
| Stage | Temperature (° C.) | Time | Cycle |

| | | |
|---|---|---|
| 1-47 - 5- | 1-2 m | 1 |
| 2-92 - 9- | 3-8 m | 1 |
| 3-92 - 98 | 8-23 s | 45 |
| 5-.5 - 65.5-15 - 45 s | | |

Amplification results are analyzed with QuantStudio™ Design and Analysis software version 1.5.1, using “Auto” setting. Ct values for each reaction are used for the qualification/classification of the run.

In order to provide proof of concept, precision, and reproducibility of the sample test kit, the Applicants performed precision and reproducibility testing for various test panels.

Overall, the acceptance criteria included: (1) Low positives: >95% with expected results; (2) High positives: 100% agreement with expected results; (3) Negatives: 100% agreement with expected results.

### Example 1: Monoplex qPCR Testing for Various Health Conditions

This example provides a non-limiting example of set up and execution of the Monoplex Assay: a set of real-time qPCR for qualitative detection of DNA from Chlamydia Trachomatis (CT), Neisseria gonorrhoeae (NG) or Trichomonas vaginalis (TV) from urine specimens which were previously analyzed by the multiplex Assay, in order to provide supplemental information on CT/NG/TV status. The method was developed to process up to 30 samples per run in a 96-well plate in the QuantStudio3 qPCR instrument.

### Amplification and Detection

### Master Mix for Monoplex Assay.

TABLE 13
Mastermixes.

| | STD mix | RP |
|---|---|---|
| PCR grade water | 5.26 ul | 4 ul |
| primers/probe mix specific | 0.24 ul | 1.5 ul |
| for target (single target) | | |
| 2 × PCR mix | 10 ul | 10 ul |
| DNA template | 4.5 ul | 4.5 ul |
| total sample volume | 20 ul | 20 ul |

### Example 2: Multiplex qPCR Testing for Various Health Conditions

This example provides a non-limiting example of set up and execution of the multiplex Assay: a multiplex real-time qPCR for qualitative detection of DNA from Chlamydia Trachomatis (CT), Neisseria gonorrhoeae (NG) or Trichomonas vaginalis (TV) from urine specimens. The method was developed to process up to 30 samples per run in a 96-well plate in the QuantStudio3 qPCR instrument.

For these set of experiments, each sample was run in two qPCRs: reaction STD detects three targets (CT, NG and TV) in one multiplex qPCR; reaction RP served as an internal qPCR control that detected the human RNase P gene. On each plate, a positive extraction control (PEXT), a negative extraction control (NEXT), and a non-template controls (NTC) were also analyzed.

In some cases, provisional reruns were performed. Decisions on provisional reruns depended on the results of initial multiplex qPCR. During the rerun, each sample was tested as duplicates in two qPCR reactions targeting the same STD target (CT, NG or TV). On each plate, positive extraction control (PEXT), negative extraction control (NEXT), and non-template controls (NTC) were also analyzed.

Mastermixesfor multiplex assay. Master mix for the multiplex qPCR reaction (i.e., CT, NG, and TV in one multiplex qPCR) was prepared as described in Table 14.

TABLE 14
Mastermixes.

| | STD mix | RP |
|---|---|---|
| PCR grade water | 5.26 μl | 4 μl |
| primers/probe mix specific for target | 0.24 μl | 1.5 μl |
| (CT, NG, and/or TV or RP) | | |
| 2 × PCR mix | 10 μl | 10 μl |
| DNA template | 4.5 μl | 4.5 μl |
| total sample volume | 20 μl | 20 μl |

Master mix for the internal qPCR control (i.e., detection of human RNase P gene) was also prepared.

Preparation of controls. NTC: Molecular or PCR grade water was used in place of sample nucleic acid for the non-template control. PEXT: Inactivated CT (LGV-2-434, Microbiologic HE0035N) or a previously tested CT positive patient sample was added to a pool of previously tested CT/NG/TV negative patient urine samples and served as a positive extraction control (PEXT) that was taken through every extraction procedure. NEXT: A previously confirmed CT/NG/TV negative patient urine sample served as a negative extraction control (NEXT) that was taken through every extraction procedure.

For the multiplex assay, 15.6 μl of Master mix (see Table 14). DNA was extracted from samples as described above. 4.5 μl of DNA (in elution buffer) or 4.5 μl of a control was added to the appropriate well of the PCR plate. The plate was then centrifuged and loaded into a QuantStudio 3 qPCR instrument. Example 3: Validation

This example provides setup, execution, and validation of a multiplex assay. For these experiments, the specimens were collected from individuals using a sample collecting tool (e.g., any of the sample collecting tools described herein) and transported to the laboratory under conditions of time and temperature determined to maintain the integrity of the target nucleic acids.

DNA was extracted from collected specimens using Zymo Research Quick-DNA™ Microprep Kit and eluted in the provided Elution Buffer. Eluted DNA was ready for input into the multiplex assay.

As described herein, the multiplex assay is a real-time polymerase chain reaction (PCR), which contains three primer/probe sets (CT, NG, and TV) targeting DNA regions from Chlamydia trachomatis, Neisseria gonorrhoeae, and Trichomonas vaginalis, respectively. The assay was run in a single well for the simultaneous detection of CT, NG and/or TV. The assay also contained primers and a probe to detect the human RNase P gene (RP) in clinical specimens or control samples. All primers and probes used in the assay were based on 5′-Nuclease chemistry (e.g., TaqMan assay).

For the validation experiments, Mastermixes were prepared as described in Tables 13 and 14. Samples were amplified in a QuantStudio 3 qPCR machine according to the amplification profile described in Table 1.

### Sample Classification Criteria

The following criteria were established during the development phase of the assay. These criteria were used to initially score the samples. These criteria were re-defined based on the validation data. See Table 15 for sample classification criteria for the multiplex assay and Table 16 for sample classification criteria for the monoplex assay.

TABLE 15
Sample Classification Criteria for Multiplex Assay

| | 1st | |
|---|---|---|
| Targ^{et} | Run: Multiplex Ct Value | Classification (score)/action |
| CT | ≤37 | CT POS |
| CT | >37 | monoplex qPCR rerun for CT |
| CT | ND and RP <35 | CT NEG |
| CT | ND and RP ≥35 | invalid, recollect sample |
| NG | ≤37 | NG POS; monoplex qPCR rerun for TV |
| NG | >37 | monoplex qPCR rerun for NG and TV |
| NG | ND and RP <35 | NG NEG |
| NG | ND and RP ≥35 | invalid, recollect sample |
| TV | ≤37 | TV POS; monoplex qPCR rerun for CT |
| | | and NG |
| TV | >37 | monoplex qPCR rerun for CT, NG and TV |
| TV | ND and RP <35 | TV NEG |
| TV | ND and RP ≥35 | invalid, recollect sample |

TABLE 16
Sample Classification Criteria for Monoplex Assay

| Target | Rerun: Monoplex Ct Value | Classification (score)/action |
|---|---|---|
| CT | Ct1 ≤45 and Ct2 ≤45 | CT POS |
| CT | Ct1 or Ct2 ND | invalid, recollect sample |
| CT | Ct1 and Ct2 ND | CT NEG |
| NG | Ct1 ≤45 and Ct2 ≤45 | NG POS |
| NG | Ct1 or Ct2 ND | invalid, recollect sample |
| NG | Ct1 and Ct2 ND | NG NEG |
| TV | Ct1 ≤45 and Ct2 ≤45 | TV POS |
| TV | Ct1 or Ct2 ND | invalid, recollect sample |
| TV | Ct1 and Ct2 ND | TV NEG |

### Accuracy

The multiplex assay was performed on clinical urine samples and the results were compared to FDA approved NAAT based assay for CT/NG/TV detection in order to determine accuracy of the multiplex assay. The acceptable correlation was set at >90%, and the observed correlation met the acceptance criterion. Tables 17-19 show the accuracy for Chlamydia trachomatis (CT), Neisseria gonorrhoeae (NG), and Trichomonas vaginalis (TV).

TABLE 17
Accuracy for Chlamydia trachomatis (CT)

| | | | % | | Accuracy |
|---|---|---|---|---|---|
| Measured | Measured | | Agreement | Expected % | Claim |
| Pathogen | Assay | N | Observed | agreement | Verified? |
| CT | POS | 46/48 | 96% | >90% | YES |
| | Concordance | | | | |
| | NEG | 108/108 | 100% | >90% | YES |
| | Concordance | | | | |

TABLE 18
Accuracy for Neisseria gonorrhoeae (NG)

| | | | % | | Accuracy |
|---|---|---|---|---|---|
| Measured | Measured | | Agreement | Expected % | Claim |
| Pathogen | Assay | N | Observed | agreement | Verified? |
| NG | POS | 49/49 | 100% | >90% | YES |
| | Concordance | | | | |
| | NEG | 106/107 | 99.00% | >90% | YES |
| | Concordance | | | | |

TABLE 19
Accuracy for Trichomonas vaginalis (TV)

| | | | % | | Accuracy |
|---|---|---|---|---|---|
| Measured | Measured | | Agreement | Expected % | Claim |
| Pathogen | Assay | N | Observed | agreement | Verified? |
| TV | POS | 42/42 | 100% | >90% | YES |
| | Concordance | | | | |
| | NEG | 113/113 | 100% | >90% | YES |
| | Concordance | | | | |

In summary, sensitivity and specificity of the multiplex assay met the acceptance criteria.

### Limit of Detection

A Limit of Detection (LoD) study was performed to determine the analytical sensitivity of the multiplex assay using inactivated Chlamydia trachomatis (CT) (LGV-2-434, Microbiologics HE0035N), Neisseria gonorrhoeae (NG) (GL0071, Microbiologics HE0041N), and Trichomonas vaginalis (TV) (GL0028, Microbiologics HE0042N). LoD was defined as the lowest concentration of each analyte at which 95% of replicates are detected.

Preliminary LoD. Triplicate samples of serial dilutions of each analyte were tested with the multiplex assay to determine an estimated LoD. Diluent consisted of negative donor urine and BD™ MAX™ UVE Sample Buffer. A preliminary LoD was defined as the lowest concentration at which each target (CT, NG, and TV) demonstrated 100% positivity (3 out of 3 replicates; cut-off=39 cycles). As shown in Table 20, preliminary LoD were: CT=24 cp/rxn, NG=2.4 cp/rxn, and TV=2.4 cp/rxn.

TABLE 20
Preliminary LoD

| CT | | | NG | | | TV | | |
|---|---|---|---|---|---|---|---|---|
| Titer | Titer | | Titer | Titer | | Titer | Titer | |
| cp/rxn | cp/mL | result | cp/rxn | cp/mL | result | cp/rxn | cp/mL | result |
| 15000 | 200000 | 3/3 | 7500 | 100000 | 3/3 | 7500 | 100000 | 3/3 |
| 3000 | 40000 | 3/3 | 1500 | 20000 | 3/3 | 1500 | 20000 | 3/3 |
| 600 | 8000 | 3/3 | 300 | 4000 | 3/3 | 300 | 4000 | 3/3 |
| 120 | 1600 | 3/3 | 60 | 800 | 3/3 | 60 | 800 | 3/3 |
| 24* | 320 | 3/3 | 12 | 160 | 3/3 | 12 | 160 | 3/3 |
| 4.8 | 64 | 0/3 | 2.4* | 32 | 3/3 | 2.4* | 32 | 3/3 |
| 0.96 | 12.8 | 0/3 | 0.48 | 6.4 | 1/3 | 0.48 | 6.4 | 0/3 |
| 0.192 | 2.56 | 0/3 | 0 | 0 | 0/3 | 0 | 0 | 0/3 |

Abbreviations: cp/rxn = copies per reaction;
cp/mL = copies per mL.
*Indicates the preliminary LoD for each of CT, NG, and TV.

LoD confirmation. Up to 20 individual samples at or above the estimated LoD for each analyte were diluted in negative donor urine and BD™ MAX™ UVE Sample Buffer and tested with multiplex assay. Acceptance criteria for LoD were defined as the lowest concentration demonstrated at least 95% positivity (19 out of 20 replicates, cut-off=39 cycles). As shown in Table 21, confirmed LoD were: CT=42 cp/rxn, NG=4.8 cp/rxn, and TV=4.8 cp/rxn.

TABLE 21
Confirmation of LoD

| CT | | | NG | | | TV | | |
|---|---|---|---|---|---|---|---|---|
| Titer | Titer | | Titer | Titer | | Titer | Titer | |
| cp/rxn | cp/mL | result | cp/rxn | cp/mL | result | cp/rxn | cp/mL | result |
| 24 | 320 | 17/20 | 2.4 | 32 | 15/20 | 2.4 | 32 | 12/20 |
| 36 | 480 | 7/10 | 3.6 | 48 | 16/20 | 4.8* | 64 | 19/20 |
| 42* | 560 | 19/20 | 4.8* | 64 | 20/20 | | | |
| 48 | 640 | 20/20 | | | | | | |

Abbreviations: cp/rxn = copies per reaction;
cp/mL = copies per mL.
*Indicates the confirmed LoD for each of CT, NG, and TV.

### Inclusivity

In silico analysis. The inclusivity/exclusivity of each primer and probe oligonucleotide sequence for the STD targets of the multiplex assay was tested via NCBI BLASTN against the Nucleotide collection (nr/nt). The database search parameters were as follows: 1) The nucleotide collection consists of GenBank+EMBL+DDBJ+PDB+RefSeq sequences, but excludes EST, STS, GSS, WGS, TSA, patent sequences as well as phase 0, 1, and 2 HTGS sequences and sequences longer than 100 Mb; 2) The database is non-redundant. Identical sequences have been merged into one entry, while preserving the accession, GI, title and taxonomy information for each entry; 3) Database was updated on 11/17/2021 (N=76216502 sequences analyzed); 4) The search parameters automatically adjust for short input sequences and the expect threshold is 0.05; 5) Default Blast algorithm parameters were used for the search: the match and mismatch scores are 1 and -2, respectively; The penalty to create and extend a gap in an alignment is set to linear. Hit results were analyzed and assessed for potential non-specific target matches; 6) For TV primer and probe blast, E-value filter of 0-1 was applied.

Overall, as shown in Table 21, the homology between available species of Chlamydia trachomatis (CT), Neisseria gonorrhoeae (NG), and Trichomonas vaginalis (TV) in the database and multiplex assay primers and probes demonstrate high specificity and the risk of significant loss in reactivity and false negativity is low. Table 22 provides CLIA validation to demonstrate sensitivity and specificity of the primer/probe design.

TABLE 22
In silico Inclusivity/Exclusivity for Primer/Probes

| | | # of | | | | |
|---|---|---|---|---|---|---|
| | | Or- | # of | Query | E- | Percent |
| | Hit Organism | ganisms | hits | Cover | value | Identity |
| CT | Chlamydia | 41 | 100 | 100% | 0.96 | 100% |
| forward | trachomatis | | | | | |
| CT reverse | Chlamydia | 41 | 100 | 100% | 0.002 | 100% |
| | trachomatis | 41 | | | | |
| CT probe | Chlamydia | 46 | 172 | | | |
| | trachomatis | | 169 | 100% | 0.002 | 100% |
| | | | 3 | 100% | 0.49 | 96% |
| | | | | | | (24/25) |
| NG | Neisseria | 5 | 99 | 100% | 0.023 | 100% |
| forward | gonorrhoeae | | | | | |
| | Hylaea fasciaria | 1 | 1 | 82% | 5.7 | 83% |
| | (moth) | | | | | (19/23) |
| NG reverse | Neisseria | 5 | 99 | 100% | 2.00E- | 100% |
| | gonorrhoeae | | | | 04 | |
| | Desulfo- | 1 | 1 | 77% | 0.73 | 78% |
| | bacterales | | | | | (21/27) |
| | (bacterium) | | | | | |
| NG probe | Neisseria | 5 | 99 | 100% | 6.00E- | 100% |
| | gonorrhoeae | | | | 04 | range |
| | Xestia c-nigrum | 1 | 1 | 76% | 9.5 | 1:73% |
| | (moth) | | | | | (19/26) |
| | | | | | | range |
| | | | | | | 2:32% |
| | | | | | | (16/26) |
| | | | | | | range |
| | | | | | | 3:32% |
| | | | | | | (16/26) |
| | | | | | | range |
| | | | | | | 4:32% |
| | | | | | | (16/26) |
| TV | Trichomonas | 1 | 1 | 100% | 6.00E- | 100% |
| forward | vaginalis | | | | 05 | |
| E-value | | | | | | |
| filter- 0-1 | | | | | | |
| TV | Trichomonas | 1 | 2 | 100% | 6.20E- | 100% |
| reverse | vaginalis | | | | 02 | |
| E-value | Siphoviridae sp. | 1 | 1 | 95% | 0.24 | 95% |
| filter- 0-1 | (virus) | | | | | (21/22) |
| TV probe | Trichomonas | 2 | 5 | | 6.00E- | |
| E-value | vaginalis | | 4 | 100% | 04 | 100% |
| filter- 0-1 | | | 1 | 88% | 0.039 | 88% |
| | Marchantia | 1 | 1 | 80% | 0.61 | (23/26) |
| | polymorpha | | | | | 81% |
| | subsp. | | | | | (21/26) |
| | Ruderalis | | | | | |
| | (plant) | | | | | |

Inclusivity study. Applicant next looked at whether the multiplex assay detected relevant strains of CT, NG, and TV. For this analysis, 34 serovars/strains represented public collections and well-characterized clinical isolates were tested with the multiplex assay. All 38 of the serovars/strains were correctly identified (see Table 23).

TABLE 23
Serovars/Strains correctly identified.

| | | | Test | Detect |
|---|---|---|---|---|
| Organism | Strain | Product info. | Concentration | [Y/N] |
| Chlamydia | Serovar D | ATCC VR-885 | 6.60E+05 | Y |
| trachomatis | Serovar E | ATCC VR-348B | 2.40E+06 | Y |
| | Serovar F | ATCC VR-346 | 1.10E+05 | Y |
| | Serovar G | ATCC VR-878 ™ | 4.50E+03 | Y |
| | Serovar H | ATCC VR-879 ™ | 1.92E+04 | Y |
| | Serovar I | ATCC VR-880 ™ | 5.60E+03 | Y |
| | Serovar J | ATCC VR-886 ™ | 1.30E+04 | Y |
| | Serovar K | ATCCVR-887 ™ | 1.30E+05 | Y |
| | AR-0166 | CDC | 5.76E+08 | Y |
| | AR-0168 | CDC | 7.69E+08 | Y |
| | AR-0205 | CDC | 8.95E+08 | Y |
| | AR-0206 | CDC | 5.44E+08 | Y |
| Neisseria | AR-0207 | CDC | 7.82E+08 | Y |
| gonorrhoeae | AR-0208 | CDC | 5.87E+08 | Y |
| | AR-0209 | CDC | 5.34E+08 | Y |
| | AR-0210 | CDC | 6.18E+08 | Y |
| | AR-0211 | CDC | 4.47E+08 | Y |
| | AR-0212 | CDC | 7.48E+08 | Y |
| | AR-0213 | CDC | 6.87E+08 | Y |
| | AR-0214 | CDC | 8.83E+08 | Y |
| | CDC Ng-98 | ATCC 43070 | 1.60E+09 | Y |
| | CDC Ng-116 | ATCC 43069 | 1.80E+09 | Y |
| | | ATCC 19424 | 3.70E+09 | Y |
| Trichomonas | 165307-1 | ATCC 30187 | 3.76E+04 | Y |
| vaginalis | JH 32A #2 | ATCC 30237 | 1.58E+05 | Y |
| | 11769 | ATCC 30092 | 2.90E+05 | Y |
| | NYH 286 | ATCC 50148 | 4.98E+05 | Y |
| | RU 357 | ATCC 50139 | 7.36E+05 | Y |
| | | ATCC 30238 | 8.30E+04 | Y |
| | MT87 | ATCC PRA-96 | 3.07E+04 | Y |
| | | ATCC 50747 | 1.20E+05 | Y |
| | HsD:NIH | ATCC 50183 | 4.00E+05 | Y |
| | TVC | ATCC 30245 | 7.30E+04 | Y |
| | NYH 286 | ATCC 50148 | 4.98E+05 | Y |

### Cross-Reactivity

Cross-reactivity of each primer probe set of the multiplex assay was also evaluated as part of the inclusivity in silico analysis (above). In particular, the in silico analysis did not reveal any organisms from urinary tract specimens with sequence homology over 80% with the multiplex assay primers and probes.

Samples containing phylogenetically related species and other organisms likely to be found in urinary tract specimens were tested by the multiplex assay at 1.00E+06 cells/mL individually or as pools. As shown in Table 24, no positive signals were detected.

TABLE 24
Cross-reactivity Measurements

| | | | Detect |
|---|---|---|---|
| Organism | | | [Y/N] |
| | E. coli | AR-0346 | N |
| | L acidophilus | ATCC 314 | N |
| | S. aureus | ATCC 6538P | N |
| | S. flexneri | ATCC 12022 | N |
| | S. thyphimurium | ATCC 13311 | N |
| | Strep species B | ATCC 12401 | N |
| | Streptococcus agalactiae (B, Ib) | ATCC ® 12401 | N |
| Pool | Acinetobacter lwoffii | ATCC ® 17925 | N |
| | Campylobacter jejuni | NCTC 13367 | |
| | Candida albicans | ATCC 14053 | |
| | Candida glabrata | ATCC 15126 | |
| Pool | Candida krusei | ATCC 14243 | N |
| | Candida parapsilosis | ATCC 22019 | |
| | Gardnerella vaginalis | ATCC 14018 | |
| | Klebsiella oxytoca | ATCC ® 49131 | |
| Pool | Listeria monocytogenes | Silliker ® SLR2249 | N |
| | N lactamica | ATCC 23970 | |
| | N meningitidis | ATCC 13090 | |
| | N mucosa | ATCC 49233 | |
| Pool | N sicca | ATCC 29193 | N |
| | Proteus mirabilis | ATCC ® 29906 | |
| | S. cerevisie | ATCC 9763 | |
| | S. choleraesuis | ATCC 10702 | |

In summary, the multiplex assay does not cross-react with microorganisms listed in Table 23.

### Interfering Substances

Various chemicals or biological organisms were tested at a concentration that may be found in urinary tract with each analyte at 3× LoD concentration in diluent consists of negative donor urine and BD™ MAX™ UVE Sample Buffer. Interference defined as 1 log unit decrease of target concentration (3.3 Ct units increase) with the addition of individual or pool of substances. As shown in Table 25, none of the substances tested interfered with the assay.

TABLE 25
Assessment of Potential Interfering Substances

| | | | Interfering |
|---|---|---|---|
| | Substance | test concentration | [Y/N] |
| pool 1 | 17-a-Ethinylestradiol | 0.48 ng/ml | N |
| | Naproxen | 200 μg/mL | |
| | Ibuprofen | 200 μg/mL | |
| | Amoxicillin Trihydrate | 30.08 μg/mL | |
| | Azithromycin | 4.8 μg/mL | |
| | Ceftriaxone | 324.4 μg/mL | |
| pool 2 | 4-Acetamidophenol | 80 μg/mL | N |
| | Human Serum Albumin | 0.4 mg/mL | |
| | Glucose | 0.48 mg/mL | |
| | Erythromycin | 24 μg/mL | |
| | Metronidazole | 48 μg/mL | |
| | Tetracycline Hydrochloride | 6 μg/mL | |
| | Trimethoprim | 16 μg/mL | |
| pool 3 | Escherichia coli | (1.44 × 10\hat{ }6 organisms/ | N |
| | | mL) | |
| | Gardneralla vaginalis | (1.44 × 10\hat{ }6 organisms/ | |
| | | mL) | |
| | Candida albicans | (1.44 × 10\hat{ }6 organisms/ | |
| | | mL) | |
| | Whole Blood | (0.4% v/v) | |
| | Semen | (2% v/v) | |
| | Feminine Deodorant Spray | (0.68% v/v) | |
| 4 | Sulfamethoxazole | 160 μg/mL | N |
| 5 | High pH (pH9.5, NaOH) | | N |
| 6 | Low pH (pH4, HCl) | | N |

### Precision

Next, Applicant assessed precision of the multiplex assay. For this experiment, each analyte was prepared at 2×LoD in diluent of negative donor urine and BD™ MAX™ UVE Sample Buffer. Three repeats from each sample were extracted and tested by two clinical laboratory scientists on three different days. Acceptable criterion was set as correlation >90% and the coefficient of variation (CV) of Ct is <10%.

In summary, positive samples for all analytes and negative samples meet criteria to show reproducibility (see Table 26).

TABLE 26
Assessment of Multiplex Assay Precision

| 2x | | | | | | | | Precision |
|---|---|---|---|---|---|---|---|---|
| LOD | Expected | Observed | | Expected | Observed | Expected | Observed | cl-85-erified- |
| Target | result | result | N | agreement | agreement | CV | CV | fied [Y/N} |
| CT | POS | POS | 9/ | ≥ 90%, | 100% | ≤ 10% | 1.6% | Y |
| | | | 9 | | | | | |
| NG | POS | POS | 9/ | ≥ 90%, | 100% | ≤ 10% | 0.9% | Y |
| | | | 9 | | | | | |
| TV | POS | POS | 9/ | ≥ 90%, | 100% | ≤ 10% | 1.6% | Y |
| | | | 9 | | | | | |
| NEG | NEG | NEG | 9/ | ≥ 90%, | 100% | N/A | N/A | Y |
| | | | 9 | | | | | |

### Well-to-Well Carry Over

Applicant assessed well-to-well carry over of the multiplex assay to determine whether well-to-well carry was below an acceptable threshold. For these experiments, each analyte was prepared at 100×LoD in diluent of negative donor urine and BD™ MAX™ UVE Sample Buffer. Three repeats from each sample were extracted and tested with the multiplex assay by two clinical laboratory scientists on three different days. Negative samples were adjacent to each other during the sample prep and PCR run. The acceptable criterion for lack of carry-over from positive to a negative sample was set at >90%. As shown in Table 27, the well-to-well carry over met the acceptance criterion.

TABLE 27
Well-to-well Carry Over

| | Expected | Observed | | Expected | Observed |
|---|---|---|---|---|---|
| Target | result | result | N | agreement | agreement |
| NEG | NEG | NEG | 9/9 | ≥90%, | 100% |

### Sample Stability

Stability of samples was tested in the diluent of negative donor urine and BD™ MAX™ UVE Sample Buffer with inactivated CT, NG or TV at 2×LoD or 10×LoD. Specimens were stored at 4° C., room temperature, 37° C., and 55° C. The stability of the samples was analyzed with the multiplex assay at the start day (day 0), and after 7 days, with 5 replicates from each positive specimen.

Samples were classified as stable if neither of STD targets in the sample (CT, NG or TV) did not decrease 1 log unit of concentration (3.3 Ct units increase) or it did not become positive (for the negative specimens). Samples were considered stable on the day when >95% of specimens remain stable. Tables 28A-28C shows results of stability at day 7.

Table 28. ASample Stability of Chlamydia trachomatis (CT)

| Stability Day | D7 | | | | | |
|---|---|---|---|---|---|---|
| | CT | | | | | |
| | 2x LoD | | | 10x LoD | | |
| | dCt -D7 - D0) ≤ 3.3 | | | dCt -D7 - D0) ≤ 3.3 | | |
| Temperature | Expected | Observed | Stable [Y] | Expected | Observed | Stable [Y] |
| 4º C. | ≥95% | 5/5 (100%) | Y | 100% | 5/5 (100%) | Y |
| Room temperature | ≥95% | 5/5 (100%) | Y | 100% | 5/5 (100%) | Y |
| 37° C. | ≥95% | 5/5 (100%) | Y | 100% | 5/5 (100%) | Y |
| 55° C. | ≥95% | 5/5 (100%) | Y | 100% | 5/5 (100%) | Y |

Table 28B. Sample Stability of Neisseria gonorrhoeae (NG)

| Stability Day | D7 | | | | | |
|---|---|---|---|---|---|---|
| | NG | | | | | |
| | 2x LoD | | | 10x LoD | | |
| | dCt-D7 - D0) ≤ 3.3 | | | dCt-D7 - D0) ≤ 3.3 | | |
| Temperature | Expected | Observed | Stable [Y] | Expected | Observed | Stable [Y] |
| 4° C. | ≥95% | 5/5 (100%) | Y | 100% | 5/5 (100%) | Y |
| Room temperature | ≥95% | 5/5 (100%) | Y | 100% | 5/5 (100%) | Y |
| 37° C. | ≥95% | 5/5 (100%) | Y | 100% | 5/5 (100%) | Y |
| 55° C. | ≥95% | 5/5 (100%) | Y | 100% | 5/5 (100%) | Y |

Table 28C. Sample Stability of Trichomonas vaginalis (TV)

| Stability Day | D7 | | | | | |
|---|---|---|---|---|---|---|
| | TV | | | | | |
| | 2x LoD | | | 10x LoD | | |
| | dCt -D7 - D0) ≤ 3.3 | | | dCt -D7 - D0) ≤ 3.3 | | |
| Temperature | Expected | Observed | Stable [Y] | Expected | Observed | Stable [Y] |
| 4º C. | ≥95% | 5/5 (100%) | Y | 100% | 5/5 (100%) | Y |
| Room temperature | ≥95% | 5/5 (100%) | Y | 100% | 5/5 (100%) | Y |
| 37°C | ≥95% | 5/5 (100%) | Y | 100% | 5/5 (100%) | Y |
| 55°C | ≥95% | 5/5 (100%) | Y | 100% | 5/5 (100%) | Y |

In summary, STD urine samples remain stable in BD™ MAX™ UVE Sample Buffer at 4C and elevated temperatures.

Applicant also provide sample stability in summer conditions or winter conditions for 134 hours (Table 29, 30). For each pathogen, inactivated cells were spiked in pools of diluent that included negative donor urine and BD™ MAX™ UVE Sample Buffer at 2×LoD (low positives, n=20) and 10×LoD (high positives, n=10). In addition, 10 negative samples were evaluated to monitor for false positives. For Winter or Summer, the samples were exposed to the profiles in Table 29 and 30, respectively.

TABLE 29
Summer Profile

| | Cycle Period | Total Time |
|---|---|---|
| Temperature | Hours | Hours |
| 40° C. | 19 | 19 |
| 22° C. | 10 | 29 |
| 40° C. | 5 | 34 |
| 30° C. | 86 | 120 |
| 40° C. | 14 | 134 |

TABLE 30
Winter Profile

| | | Total |
|---|---|---|
| | | Time |
| Temperature | Cycle Period Hours | Hours |
| −10° C. | 19 | 19 |
| 18° C. | 10 | 29 |
| −10° C. | 5 | 34 |
| 10° C. | 86 | 120 |
| −10° C. | 14 | 134 |

The results of the summer and winter stability studies are shown in Tables 31 and 32, retrospectively. Samples were classified as stable if neither of STD targets in the sample (CT, NG or TV) did not decrease 1 log unit of concentration (3.3 Ct units increase) or it did not become positive (for the negative specimens). Samples were considered stable on the day when >950% of specimens remain stable.

TABLE 31
Results of Summer Stability Study

| | | | Ob- | | 0 h | 134 h | | Is Ct |
|---|---|---|---|---|---|---|---|---|
| | Agreement | | served | Acceptance | Aver | Aver- | Ct | increase < |
| Sample | Acceptance | | Agree- | Criteria Met? | age | age | In- | 3 |
| Type | Criteria | | ment | (YES/NO) | Ct | Ct | crease | (YES/NO) |
| CT in urine | 2x | ≥95% | 100% | YES | CT = | CT = | 0.03 | YES |
| (BD max | LoD | | (20/20) | | 34.83 | 34.86 | | |
| UVE) | 10x | 100% | 100 % | YES | CT = | CT = | 0.35 | YES |
| | LoD | | (10/10) | | 32.43 | 32.78 | | |
| NG in urine | 2x | ≥95% | 100% | YES | NG = | NG = | — | YES |
| (BD max | LoD | | (20/20) | | 34.95 | 34.54 | 0.42 | |
| UVE) | 10x | 100% | 100% | YES | NG = | NG = | 0.4 | YES |
| | LoD | | (10/10) | | 32.56 | 32.95 | | |
| TV in urine | 2x | ≥95% | 100% | YES | TV = | TV = | −0.7 | YES |
| (BD max | LoD | | (20/20) | | 31.53 | 30.84 | | |
| UVE) | 10x | 100% | 100% | YES | TV = | TV = | 0.35 | YES* |
| | LoD | | (10/10) | | 28.77 | 29.12 | | |
| Neg urine | | 100% | 100% | YES | N/A | N/A | N/A | N/A |
| (BD max | | | (10/10) | | | | | |
| UVE) | | | | | | | | |

*At 134 hours, one sample of 10x LoD TV Ct increase is over 3, but above detection limit of TV. Should not change detection status of TV positive samples.

TABLE 32
Results of Winter Stability Study

| | | | Ob- | | 0 h | | | Is Ct |
|---|---|---|---|---|---|---|---|---|
| | Agreement | | served | Acceptance | Aver- | 134 h | Ct | increase < |
| Sample | Acceptance | | Agree- | Criteria Met? | age | Aver- | In- | 3 |
| Type | Criteria | | ment | (YES/NO) | Ct | age Ct | crease | (YES/NO) |
| CT in urine | 2x | ≥95% | 100% | YES | CT = | CT = | 0.5 | YES |
| (BD max | LoD | | (20/20) | | 34.74 | 35.23 | | |
| UVE) | 10x | 100% | 100% | YES | CT = | CT = | 0.09 | YES |
| | LoD | | (10/10) | | 32.86 | 32.96 | | |
| NG in | 2x | ≥95% | 100% | YES | NG = | NG = | −0.51 | YES |
| urine (BD | LoD | | (20/20) | | 35.34 | 34.86 | | |
| max UVE) | 10x | 100% | 100% | YES | NG = | NG = | −0.41 | YES |
| | LoD | | (10/10) | | 32.92 | 32.51 | | |
| TV in urine | 2x | ≥95% | 100% | YES | TV = | TV = | −0.3 | YES |
| (BD max | LoD | | (20/20) | | 31.26 | 30.96 | | |
| UVE) | 10x | 100% | 100% | YES | TV = | TV = | −0.44 | YES |
| | LoD | | (10/10) | | 29.25 | 28.81 | | |
| Neg urine | | 100% | 100% | YES | N/A | N/A | N/A | N/A |
| (BD max | | | (10/10) | | | | | |
| UVE) | | | | | | | | |

In summary, the acceptance criteria was met for each sample type under both summer and winter conditions.

### Example 3: Multiplex qPCR Testing for Various Health Conditions Using a Combination of Samples

In this example, multiple samples from the same user are combined as a single input for qPCR analysis. These samples are various specimen types collected from the same user: e.g. urine, rectal swab and oral swab et. al.

Once the samples are collected, dry swab samples are rehydrated as (1) a single swab containing one sample at a single location from the user (e.g., oral, or rectal sample). Rehydrated sample solutions from each matrix are mixed before PCR as a single qPCR input or (2) dry swabs (oral swab, rectal swab, etc.) are rehydrated in a single container. The rehydrated sample contains multiplex matrix and is used as qPCR input to determine STD status. Examples of specimen multiplexing in comparison to single matrix qPCR are shown in Table 33 below.

TABLE 33

| | Single matrix qPCR input |
|---|---|
| 1 | urine |
| 2 | rectal swab |
| 3 | oral swab |
| | Multiplex matrix qPCR input |
| 1 | urine + rectal swab |
| 2 | urine + oral swab |
| 3 | rectal swab + oral swab |
| 4 | urine + rectal swab + oral swab |

The multiplex sample matrix does not compromise assay performance compared to a single sample matrix.

### EQUIVALENTS AND INCORPORATION BY REFERENCE

All references cited herein are incorporated by reference to the same extent as if each individual publication, database entry (e.g. Genbank sequences, GeneID entries, or UniProt. ID), patent application, or patent, was specifically and individually indicated to be incorporated by reference in its entirety, for all purposes. This statement of incorporation by reference is intended by Applicants, pursuant to 37 C.F.R. § 1.57(b)(1), to relate to each and every individual publication, database entry (e.g. Genbank sequences, GeneID entries, or UniProt. ID), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

While the invention has been particularly shown and described with reference to a preferred embodiment and various alternate embodiments, it will be understood by persons skilled in the relevant art that various changes in form and details can be made therein without departing from the spirit and scope of the invention.

1. A multiplexing system for detecting whether at least one sexual health condition is present in a user, the system comprising:
(a) a collection kit comprising:
i. a sample collection device for receiving one or more samples collected by the user at-home or in a clinic;
ii. a sample transport container designed for receiving the one or more samples collected at home or in a clinic; and
iii. a labeled shipping container for shipping the one or more samples to a laboratory for analysis;
(b) a laboratory analysis kit comprising:
i. an amplification reaction mixture; and
ii. primers selected from:
a first pair of primers that bind specifically to a target nucleic acid sequence from Chlamydia trachomatis where present in the sample,
a second pair of primers that bind specifically to a target nucleic acid sequence from Neisseria gonorrhoeae, where present in the sample,
a third pair of primers that bind specifically to a target nucleic acid sequence from Trichomonas vaginalis where present in the sample, or
a combination thereof;
(c) analyzing the sample using the laboratory analysis kit to detect whether the sexual health condition is present in the user; and
(d) a computer-readable medium storing code that, when executed by a processor, causes the processor to perform the following steps:
receiving and displaying results of the analysis with the laboratory analysis kit to the user, the results providing information about whether or not the user has the sexual health condition.

2. The multiplexing system of claim 1, wherein laboratory analysis kit further comprises an extraction and/or storage buffer.

3. The multiplexing system of claim 1, wherein the analysis comprises means for nucleic acid purification and amplification.

4. The multiplexing system of any of claim 1, wherein, if at least one sexual health condition is detected in the user, the processor is configured to further perform the following steps:
provide telemedicine access to the user with a healthcare provider, wherein telemedicine access allows for communication with the healthcare provider for running tests, displaying test results, providing telehealth visits, prescribing and facilitating delivery of a medication.

5. The multiplexing system of claim 1, wherein the sexual health condition is an infection associated with Chlamydia trachomatis.

6. The multiplexing system of claim 5, wherein the Chlamydia trachomatis infections are associated with Chlamydia trachomatis strains selected from Serovar D, Serovar E, Serovar F, Serovar G, Serovar H, Serovar I, Serovar J, Serovar K and LGV strains.

7. The multiplexing system of claim 1, wherein the sexual health condition is an infection associated with Neisseria gonorrhoeae.

8. The multiplexing system of claim 7, wherein the Neisseria gonorrhoeae infections are associated with Neisseria gonorrhoeae strains selected from AR-0166, AR-0168, AR-0205, AR-0206, AR-0207, AR-0208, AR-0209, AR-0210, AR-0211, AR-0212, AR-0213, AR-0214, CDC Ng-98, and CDC Ng-116.

9. The multiplexing system of claim 1, wherein the sexual health condition is an infection associated with Trichomonas vaginalis.

10. The multiplexing system of claim 9, wherein the Trichomonas vaginalis infections are associated with Trichomonas vaginalis strains selected from 165307-1, JH 32A #2, 11769, NYH 286, RU 357, MT87, HsD:NIH, TVC, and NYH 286.

11. The multiplexing system of claim 2, wherein the extraction and/or storage buffer comprises phosphate buffered saline, Tris-buffered saline, HEPES buffered saline, a reducing agent, sodium hydroxide, Triton X-100, octyl glucoside, PreservCyt® solution, CytoLyt® solution, or a methanol-based reagent.

12. The multiplexing system of claim 1, wherein analysis is performed as a single reaction or in a single reaction container comprising the amplification reaction mixture, the first pair of primers, the second pair of primers, and the plurality of target nucleic acid sequences from the one or more samples.

13. The multiplexing system of claim 12, wherein, of the first pair of primers and the second pair of primers, each includes a probe, wherein the probe binds specifically to the respective target nucleic acid sequence.

14. The multiplexing system of claim 13, wherein:
a first forward primer of the first pair, a second forward primer of the second pair, or each of the first and the second forward primers comprises a concentration ranging from 0-05-1 uM; or
a first reverse primer of the first pair, a second reverse primer of the second pair, or each of the first and the second reverse primers comprises a concentration ranging from 0-05-1 uM.

15. The multiplexing system of claim 6, wherein the first pair of primers comprise a forward primer having a nucleotide sequence selected from SEQ ID NO: 1-19, a reverse primer having a nucleotide sequence selected from SEQ ID NO: 20-35, and a probe having a nucleotide sequence selected from SEQ ID NO: 36-47.

16. The multiplexing system of claim 8, wherein the second pair of primers comprise a forward primer having a nucleotide sequence selected from SEQ iD NOs: 48-59, a reverse primer having a nucleotide sequence selected from SEQ ID NO: 60-70, and a probe having a nucleotide sequence selected from SEQ iD NOs: 71-84.

17. The multiplexing system of claim 10, wherein the third pair of primers comprise a forward primer having a nucleotide sequence selected from SEQ ID NO: 85-95, a reverse primer having a nucleotide sequence selected from SEQ ID NO: 96-104, and a probe having a nucleotide sequence selected from SEQ ID NO: 105-115.

18. The multiplexing system of claim 1, wherein the collection kit further comprises a sample collection tool selected from a group consisting of: a collapsible cup, a tube, a pipette, a swab, a dropper, and a combination thereof.

19. The multiplexing system of claim 1, wherein the one or more samples are selected from a group consisting of: a urine sample, a rectal sample, a vaginal sample, a penile sample, an oral sample, and a blood sample.

20. The multiplexing system of claim 19, wherein the system comprises a combination of samples, wherein each sample of the combination of samples is collected from the user using a separate sample collection device.

21.-88. (canceled)
